SEQUENCE LISTING (72 sequences)

SEQ ID NO: 1 - RNA, 35581 nt, synthetic construct
  source 1..35581: mol_type = other RNA
aataaactggaaagacgcctggtctggcttcagttacagggagcaccaccagggaacatctcggggagcctggttggaagctgcaggcttagtctgtcggctgcgggtctctgactgccctgtggggagggtcttgccttaacatcccttgcatttggctgcaaagaaatctgcttggaagaaggggttacgctgtttggccgggtgagttttattggcaaactgtgcctctgggtgatgtgtgcctatgctttacaagaattgcctaatttcccaccccctgcaagccgcaaatgaaaaggattgcaggagagatggtgcatttgtgttggaattgactggagattcagagggctttttatatccttggttaaaaggtggatatatactctggctggggaggtggggggccatctgagagcatctagaaaccctaatgcatccagattgaaaggaagaaaggaatctagatgctttttcccccatggaaaatagctgtgcacacacagctggcaggtggccttggtaagcaggttagggagaagctgccacctgtggcagagccttggccagccgggctctgggtgtggcaagcctggtcagcaggcactgagtagcacttcctctgccaccattgagaaccaggcaggaggccagaggcagtgagggacagatgggttgggtttacctgtctggcagtatatggtgtggctgtgacctgggtggttattgattaaactattgggttcaaaagaaaggaagaagcgagctgtagcacccaatatatgcacttttctgtatgtgtattagactttattaaaaagtttatttgaaaatcacaaaggaagggaaagaaaaccctgagttagatatgctcatatttctaaagtgcttacttagaacagtcctgagtatgttgtgatcacataagtgttggttaaataaataaatgtccaaatggcataaaacaaagtaataatttctagagcaaatctaacttataaaatgacctgtggaggaaagaaaacactgccatgtctttagacttttttttctacttgcatatgcactttcatttataaatctttatctatctatctatatctatctatctatctatctatctacctacctacctacctacctatcagagttacaagctactttagtgcagaggtggtcaggtcttccctgaaaggttgatgtgagagttaaatgtgatataataatagtggtggcaatttgggcactgggcccttattatgttgcagacacgatgttaactgctttgtacatattgtctccttcaatcaccaccataaacctttaaagcaggcattactatttccttttgcacctgagaaaactgaagctcagtgaacttgagaaattgcccaaattcacaacaaaagtaagcagggtgattaggtttgttggagcccagagccagagtgcctaaccactgcaccatactgcctctcacagaccatgcatataaagtccaggcaaagtgcttggcatataacgggcactgtaaatgcagtgtttacaagtacacttcattttaatctgataacacgatagttttaaaaagatcattgtttgaagatctttttcaaatttatttttacttaccgtaagaatatacaattagctaaaatagcagctcctttcaaatcgtaaaataatatagaattatttgaatcattgagagtgatgagcttctatgacacagtacactagaggtagggaagtgtgtgtgtgtgtgtgtgtgcgcgcgcgtgcgtgaactgctgcattttcaggcagaaacctttaacatccacattcctgctccctgtcccgtgcctcaaggctggcctgcgcacgggagtctcagttgggcgcgcctctgccaagccgagactgaagggggctagcctctctccctgtaacgctgggcgggccacgttaggaggctatgaatcagctgatttccttggctgctccaaccccacctcaaatggccacctcgcacccgcccgccaaaccccatggccaggactccagccaaggctgacagccaagcccgactgctgcaggaacactttgcctagagcttattcggtggtagtctggttttgcctaggctaggaggagcccaatcccagaccatgctatccagtagtcggccggacttttcttcccctaattcgcacccaagaggagcccgctagatcaatccccgccaatcctaggaagctggcatgcttcgtaggtgcagacagtaatagcggggaccggcgcggggcaggtgtgtccggctaagacgccaggacagggcaggggctccaggacccgagaggagagggacttcttccagcgctcaagcgcccgctgccctctattagtgggaaaggaatccagcctcagccccgcgcgggcgcggtcggcgtcggcgggcccagaagcccagccctgggcatccgctgagctacatttggctgggtcttcccagagtgggctgaggagccagtttctcggtcaacactaggtctccacggggccaggggagaagggaggtgggaggtgagaaagctcagccgcctctggtttcgagtaaaagtcgccgcggttttgcagggaccgactttttcttgaggcgcatttaaggccaagtgactgtctcctgccctccctctctcctgccccctctcctccctgagtcccgccctcccgcacacgctgacccagggacacaccctactgcagcgacgcaaacagggcgttgttcccgttaaaggggaacgccaggagcctcccactgcccccttgcttcgcgcgcgcgcagccccgcagcgcagctttggcggcgccagcagcggagccaacgcacccgagtttgtgtttgaggccaccctgaggatcgggacagctgttcctttgggctgtaagtgatttggtggggagagtgaaggagatggaggagaaatgtgggcatcttccagtgagggtgccagaaagcgcagcgcaggcgcggggctttggccagctccctggggcttctgtttaggggcacagggtcccctgtgtgtcctgtttccctccaatgggtcttggagtaatacaacgaggagagcctttatggtctataagaccttacagggcaagggtgatggtgctggtgtctggatagcggataagggcggcccagttctcgccttgctgaagaggcgttgactctgggacacactggcaaaacagtccctgtggcggacatcccgagagtgttggactcctgtggttccccagttcccagcctcctgccatcggcttaattcaaaccctctgggagtcatcagaaatccttgtttatttcttccagtgcattccagagtttctttgaatgtgatgcttgtggagaggaacagaggggcctgggattgggtactttccaaactgggttattgaaacgttgtctccctggcccaagactctgcctaaaatggtccccagagtctcagccagatctttcacagccatgcatgtcctctaacaggtcatcccagccttcagttcccccaaagtttaaaatgaggggcgaggaccgcattacttgggataaatgtcctgccggctctgaccggctgaaattctcggactcagccttttaccctctgtctctctctcccgtccgctggacatctactcccttcggtccaggctcctgggctcctgtcccaccgcacccagaccggagctcaggcttgttggggttcgtgtcctggctttgagtgcctggggtgcagactggacccctaggcgagcacgtgggtctagcgcaagagcagaatgccccctgaccccaggggctgcctggggaagcgcgcggtggacgggaagcgcagggtccgggcaaccctctcgagccattcttttctttccacactactctggctctgcaccacctctccggagccgccagagtctgcgcgaggctgagctggggccaggaccgttcctctacgctggcagagttgggtggaaacttggagacaaacggaatgcggagagcactggggtctgggaaaccagcctcctcgccgctgtctcccccaccgcatgccacggctgtctccaggtctcagacggaaatctgggcacctactcccctcacccctacctccaccccataggggaattcaccatctgaaccggggtctcggagaacgtgacctctcaccttccagggaggtcgccgggaggtgcttggggtgggtggaagcgtgcagtggcctctgctcatattttctggaaacccctccgttccctgggtggttttagacgtgcgaaccgcttgttttgtttccaaaagcaaaagatgttccgttgcaggcgggcccggctgggcgctgggcactgggcgctggtcctgcaggcggctgctgccccctctcggcggcaggcggcgcgaaggctcctgacccgcgcgggcggtcgggctgcgggcgctgggccaggccgggccttccgctagtgcgcgggaccctccctctgcgcgcgcctccgtcgctcggcccagtgcgttcggcctcacgcccagcgctctcctcgcaggcagaaactccgctgagcagaacttgccgccagaatgctcctcctgttgctgagtatcatcgtcctccacgtcgcggtgctggtgctgctgttcgtctccacgatcgtcagcgtgagtgcctggcggggaggctccctgcgcggcccgcccttccccatctgggttcccagcccgtgctcctgctggttcaggactgtgttatttgcagacagttggaagtctcagacgtcccagggaagtttctggcaatctgcccccttccagttgctttgagaaaacgagagcagattcagtgataggaaccaagcgagcgctgggctgggttagctgcgcaggtctctatttaagccaagtaacttcagagccgatctagggtccccgaccttcatttgacaagattgtttaacttttttttttcttgatgcagcctcgttgaagaagaggagtatttatgatttttttttctccaaaccatagtcagatgggtgaattaatttataaaacacccttttaggacttgaaattggaaagttaaaatgggcttttctggaaaagagttcacaggtcccatgtcggctttatggcatgaacacaacacattgttagtaaagcttccactggtaggaacaggccatcagagtaacttctgttacaaaactgtccagcccattagatcttaaagttattttcttggcgatgaagatgagcagacattcaggcagttttccagtggtggctttcacttcttaatttaaggcacattgtaagcatgattttctttttcgttttcttttcttttcttttctttttttttttttttttgagatggagtctccagcctgtcacccgggctggagtgcagtgccatgatcttggcttactgcaacctctgccttccgggttcaagccattctcctgcctcagcctgggattacaggtgcccgccaccacgcctggctaattttttttttttttgtatttttagtagagacggggtttcactatgttggccaggcttgtctcaaactcctgacctcatgatccgcccacctctgcctcccaaagtgctgggattacaggtgtgagccaccgtgcccggcctgtaagcatgattttctagtctctgtgagaaataattatgttggggatttgtgacttagtttaacatttacaagcatcttcacagtatctccattgtgcctggtgatgatgatatcagactgataggagctataaacaaggaaatagactgagagaggcgggcttggggaatgatcccgagatgctgggaggagagaaggcttgaatgcaggtgtctaaggttgagttcatggctccttctactatagtaaacatctctgcacataatcgtttctgtgtgcatgtggaacttctccatttacaaggtgcttttaagtcataaaacgttggctcttaccatgcaggggtgggcggtgtggctaggtggatgcgggtgcttttcgccatccctgggcctttctccttccccttttccttcactcctccctccctccctgactcaggatatctatctgattctctctagcaatggatcgtgggcaatggacacgcaactgatctctggcagaactgtagcacctcttcctcaggaaatgtccaccactgtttctcatcatcaccaaacggtgaggctggttttgtgctccatgagcttgtcctcagacgcctgtaacacgtttctcagcccagagctggaaacgcttattggaagacacaagccagaatgtcgttgctggggtggggtgggatgtgacagggagccatgagtccagggaaaatgggaggggtggtgatctcgctggggaagctgagatcctgggcatcattgtgattcactctacctagagacatgcccttagtgccctcctgatcacttaggataatgctttttgtgattgaaaaaaatatcgacctgggagctgtgaaggtggtatgatagctaagcctttggcagacccccttgggacattggatcaaacgtgtatctttggtttggtggctgtctctattcagaaatgtcctgcccctttctgctgcagtgacagtggggctagctcactgcaccgtcagttacatggacacaagactggctgcctttccaggtcttgggcctcagtgatgccacctagcatcaaaacctttgaatgtttccttgttgcaagatggggacttgaagctatttgcaaaaccctaagctaagccttggggtccttaatccagaagatcgtatttctcctctgtcctcagcatctttgcttgtgagatatggaagccaggcttcatgagggtcaaattaagggataattggcagcagaagcaccaactaggtacccaaggcacccagagggaaaggaacacccagccaggagccctgtgtgtttggatgcagacaggtggaggtaaacaggatgagctgttttggtggctgggtaggccatagcgattgatgttaagagcccgtggacctggatccacatgatcttttgttccagaagatgtgtcctcaggctggaaggacctagagatacagcagagctttcctaacatcctgggtttaaaaggccacggaaatacagctttaaatgttgctggagctactcatagatccagtatatatgttcaagatgctacaccagacgagagaaaccagtttagatccagttcagtaaaacaggcagtgaagtggcctttggtcctggctttgcataatccagttgctttgctggactttccctcttacagctcagtacccagtaatacccaagcatgtccccagctgaagcattataaaccaaattccttgaaccacagtacaaatagaaactgctaaaataataatttggtaatttacatggagaagatacgcaaggagtagaaaaccctgctttatgtttttaccttctgggttggggagggagcagagaaaaggtcccagtcctattggagtgagaacagtgcttaatcttgtctctgagtgtgtgaaaggcattgaattaaagccatcccagaaattacgtggtgggtttgcatggtgagttggcaactgagtaaatgaaatactcatgagtgcctgagaatgtgaccgtagcactgcttgtccccaaagccacagcatccggtttctccccctttagggtctggctgaagttcccggagcactccggatgggagtctccgtactcgctactgacacctggtggttacaagtagtgactcagcccgaaaagggcaggctttggtgctcacaggcgccatccccaagtggcatctggccacgcggcttggcacagaatcctggactcttgggcccggaagggggcagttttggcaggcgtgttaaaacccggcgaagtttcagcagaaacgaggcggcagaggagttgctaagttgtgcttaaaccatctgcaggaaagaaagcaatattgacctgtagcatgttacagattgacatttggtgtcttctgccgttggaaatagctgtcagttggtgctagaagcagatctcaaagaggcactagggttattttaatcaggaaacttcttatctctcaacttgttccttctgtgccaagctcatgttcttgagttcatttacaatgcctccttagtgtgggtttcaaatctgcctcgttgctttccgtgaggaccccagagtgtctgttttccacatctcccttctcagctctcacaatcagggcattttgaaaacatctggacactgcacctgaacatgctgggtttgttttcacgccactcaggcttaatctaaatttgaaatttccatttacacttccccagtagtggttatctctgccgctatcttccccaacgtggcagcacttgctacgacttcagccttaagtggcgaatcctccagggcctctttgattgagtttaacaattgtggctgcagacaataagctgaaaaaaatgtgtactttttaaaacattatactgtctttacaaatgaactatgctcattgcgaaccatttaacttgtctgtatttgctcttagaaagtatatttgaaaaaaaatacttgttaagtaatcaaaagtaatttgttattgacatcgatttgtgaagaatatgttgcaagtgtgaaaaaataaatagtggtgcaatctcggctgactacaacctccacctcctgggttcaagcgattttcctgcctcagcctcccgagtagctgggattataggcgcccaacaccacgcctggctaatttttgtatttttagtagagaggggtttcatcatgttgggcaggctggtctcgaactcctgacctcaggagatccacccacctcacctcccaaagtgctgggattagaggtgtgagccactgcgccatgtgacttggtatttttttctgaaaggactcatttttctggtgcatgaccatgacacttcacctctctctgggggttgccactcatgttgttttctacatgaatggggcctcctggcatcgtgccatgaggcagtctaacatctaggttgtgtagtttgaaagagacacaccagatcttgcccccaggctacatcttaatattagaaatagttttagatttttaactaactgactttgactccgcctgccttctcgtttttctaaatgtgtttgtatactttctggcaccccctccttatctgcctcgtgtgggcagaggtaagagaggcttagtgtggggatattcgttatgatattgtatccagtgcctctccggcccagggaatacaaggatctggaatgcatgggccttgccttgatggtcccgagcctggtcgaagagtcaggagatagatatggaagggtgaatcactcccttggaaggcagagcaggccaggtgcccaaagggaggcaggcagacagcgtctgagttcagggaggaagcggatgggaaatctctggtgggaggtagaatctgatccatcctccaaaggatgggcaggatttaagtcaatacaggcaacggggaggagaatattccctgtgacctaaggtaagggtagaacaaggcaggggatggggtagactggttggctagtgttctggttaggattaggattgattgtgtataagagaacatcttcaaatgataggggcttaagtaagatcatttatttttctctcttttcaaagaagtctggaagtaggtagcctgagagaggtatggaggttccatgaaattgtcagagatgcagacactttctagctcatttctttgccatctcttgggtttggctgtcatcttcaggaaacaaaaggctgctagagctccagccatctcatccatgattcaagcagtagggtggagaaaggaggtggggaagaatgaatctccttttcttttaatgaaatgtgacatggtcacacctacacctatttacaagagaggctgagaaatatagtcattcggccttgggggaagcatggcttttcaaatcatagaaacctggcagcaaatacctctctgtaagcctggtgtggaatactttccttgagggtttgaccatcccctcccatacccaatcatactcttttgcaatagctcagttgggccctatgcagttccataagtcccagttatttcagcattcttcaaagagtccatctctgctgcatcctgggctctgtcagccaccccaccattctcacatgggctcttctcagaaactttaggacctgttgaggcttctctctctctatttctaccacctctccaggtataagtaagtcttattattattattgagacagagtttcactcttgttgcccaggctggagttcagtggcgccatcttggctcactgcaacctccgcttcccgggttcaagcaattctcctgcctcagcctcccgggtagctgggattacaggcatgcgccaccacgcttggctaattttttgtatttttagtagagacgggctttctccatgttggtcaggctggtctcgaactcccgacctcaggtgatccacctgcctcagcctcccaaagtgctgagattacaagcgtgagccaccatgcctggccatcttattattatttaatgagtccagacctctcttctgaatcccagacctgtttatccagctgtctcctggccacttcccttgggatatctaagagaggcccccaagtcactaggccagagacagtccttatcttcctctgccaattcaacccctctgtcactttctagtattttagtgaatgctgcttcaccagccagcaagccaaacatctagagtctgccttgacttgacgcgtttccttctttctcttctcagcattgtcacgggtccctcaggccaccccattgttccaatgcctgttgcccacctgtggtcacgctgcttcctcgtttgtcagaggcgccagggcagcctcctaactagtttgcctgaaccctttcttgctcctctgtgactcatttttcatatggaatccaggctgatatttaaaaaagcacaaacctaattatatcactcctcattttgaaacctttcagaggccgttcattggtcttaatcttcagatgaaaatctttaacaaggcccatcggaatctacctgaatgatccactcctgcttctgttaagtctcacctcccatctctcttcccctcatcttggaatttcagcgttttctcacgtcctgaaatgtgaacttcccacttcgggagtctctccatttctcttctgatctttcatgcaggactatttcttgaacaactctcgtgtaccagagactattgcaggtgctaaggttacaagagtgaacaagagcttatgttttactggacaaaccagtcagtaaacatggagaatttgggggcaatggtaagaactatataagacagggtaatgggataaagagggattgcagtggtgaaggaaggtctccttgaggagtggccaggagccagccatatgaacatctcggggaggttccatacagaaggaccgtcaagtgcaagatcgctggggagggaaggagcttggcgtagttgagggacagagggcagggcagactagtgggaacagagagtgaggggtaagtagaatcacagaccatctcaaaggtaccccccacccccactaggaaggagttgggtgcacatggatgcatttctaatctttgtgatttccttagacctgctgtcttccacgggtttctcagtggtccatttgattccaagttggagtgacagtccagaagcctctataaggcagaatatatcagaaattgtacaaggtagaaatgcatcggtatattttcagtgattattttgggtgacgggattcttatcactttggctgttttctttcttttgctgccttacctgttttcctcattgagatcttttattttcacaatcagaaaataactgtgaacattttatgttagaaaattggttttgagtagtaagtaggttccctctgccttgattaagagagtggagccctggggcatggcacgcagggtccatggcgtcgctttgaatgaggccagttcctggagctttagtttgcaaactcatacaatctggaaggctctggtgagggggaggggtgtgccaagcctcctccccaagaccctatgggtgagtttaaaatctactgacccaaaggattcaggcaaaacatttgccctgctattgaatgaaccagtgctttttgttttgaagtctccaagatccaaatatcaagtccaagttctcacaagcattcccctgctttttgtttaatttgtgtggacctgacgtgtagcagaatgggtcagaatactggaattaagccacaggcggcttgggagcccaagagctagaagggcacagtcctggcttgccctggcccctggctggtcttcatcagcttcagtgatgaggcaggacccaggcagtttgtcttcagtaaaatctgtctctcagggcctgtccctgctcacaaagtcctctcttgtctcagaagaaaaggtagttgataaactttttcatgctgaggtgttaggcacaaaaaatgtgcccctttttgaatcacttttgtttttaaaaaggaggccagtgctcaacaccaaagcatgaagggaattgttgagctttcctgtaatctgtccccaaacgtccttggccacctccattgtctaagggcagtacctgcagcccctcatttcccagtgacaacaggtgggtctgctctgccacagtgtgtgggcgggaccagcaccagacgctgagaagatgaccacttactactcttcttttccatttaaacagcagcagcagcagcagcagcagcagcagcagcagcaacagcaaactcatgactatcaataggttgtgtgaggaattagatcaatgctttgggttgaaaattagagaaactaaatcttgccagtctcagcagcccccaacctgagcccttggtggtctcctatcatcgctgtcttcaaaacgatcccagacttgtttatcaatttggtgacactgtcattttggcaaggacaccagacaatttccttgtctgtggctcagcttttggccagattttagaaaacacgggagaccttctgcatttatgggttcatgttttacattttaaaatcttcgtggctccctcagccaacccataatctctacccaaggaatggctctggattttacaggctcttatgagatgctgagaccctggtggtacaagggaggtacagaggacggaatgtgttgtcaacatgccaggatttcaacccttagggtttctctgatgccaatacccaggaattagtatgaaatgtttgtttgaacctgtgtctgaactgtatgacctggaatctatagtctttgctttaaaaaaacttcaattgtatccccaagatttcaccagcaccaagaaaacatcccttgcatttcaggagcagggactggccatggtgcagcctgaggttgtggatgttgatctttggtgcccgagagaaggctgtctgggatgaagccttctgactgtggtcaggtccctgccagtgctgggggccacatgagtgtgcagtcatccacacacaagtggcccccaacactggcagggacccggagtcatctggcccatccctccttcctatcccatcgagccacagacccctctgcaatatgcctaccacatgttcatcctaccactgatggggaacttattaccttcaagagagcctcttttgtttttgaagagctaatagtgagatccattcatgtgttcttcaactctattgagcctgtcctatgtactgggcactgttctagaaatggggtgcacagagatgaacaagaaagagcaggtccctgccattgtgcagctcacaaggagttggaggaaacaggcaatacacaatacatgcatcactatgggtagtggaacatgctctgcaggaaacaaatagggtgatccaatggaaagtcatcactggaggaatgagctgctcagggaaggcctcttccttccatagtgatgtggtctgagatttagaagacagggaaggaccagttataggaagaaccatgggaagaaaattctaggaggtaaagggaagaaccaaggccctgagttagctgaagaaaggagggtggggaatgcgggagagagggatgggagcaggatcgctggtgcctcacaggctgtggtactgagaaggacagttgggacatcattgaagggttttaagcagaagagtgtcatgatttgattcatgttttgtaagctcattctggctgttgagaatgaattctggggagaggcaagagtggaaactggggaccagtgaggagtttgtggtcatagtccagactggacatgatgatgacactgagatgacaatgatgacaatgacaatgatgttaagaatacaaacagctcacgtaagtgccaggcattctttgaaaagcaatgcatttaatcctcacaagaactctaggaggtagctggtggcttagctagggaggggaagtagagaagaaaacaggtctcatgacagggtttgctgatgggttagatgtagggagttagggaagaatgattctagatgactctacgttttgggcttgagcaactggtgtgtaactgaatcttggactgagatggggagactggaggaggaacagaagatttgcgggaaggtgtagaaatcaggatttatattttgaccatcagagaggtcagcaaatcacttcaatttagaagtctttggcttaggggagaggtctgggctggaaattcaaagtcaagagtcattcctctataccgaagggtattcaatgttgtggtgctgaatacagttacttggggaggagttgagagcagaaggcagaaaaggggaacagggttcagagctaagtcttgggcatgccaggggttaacgagggtacaggaaggagaggagactggggatgaacagccagtgatgagggaggaatgccaggagatattgtcacttaagaaattacctcggctgggcgcggtggctcacgcctataatcccagcactttgggaggctgaggcgggcggattacaaggtcaggagattgagaccatcctggctaacacggtgaaaccctgtctctactaaaaatacaaaacaaaattagccgggcgtggtggccggtgcctgtagtcccagctactcgggaggctgaggcaggagaatggcgtgaacccgggaggcggagcttgcagtgagccgagatcgcaccactgcacgccagcctgagcgacagagtgagactccgtctcaaaaaaagaaattaccttgttaaacccaacactgcccctcttttgtccattcacttctagttgccttcattcatgcctgatgtgctggacttgggttttatcaagttgcttggcacgtccttaaagaatttaaatctccacagtgcctcctggacaatgactgcagggtgacccccgtctctcctctacacgatgactcttcaaacagttgacgattgcagtttttctttccttccttctttcagtggcttctctggtgctggtcccagctgcataagcagggcctcttcttgcccttctgaagtttggtcagtggtttggcttggcaggctgatgagcagagaataagggaagccgctctctctttgccacaccattctccttccaccctctctagtttttgtggattctaagtctgaagaatggtggtagaataatttaattcattagattgcaagcatatcattgacttctctgcacccccagtgtgttgtagtggaaaaactagtagtctgggcatcagggaatccttgtttttctgcaccgggtctgcctgtaacaaattaggtaacctggggcaggtaccctggtttcctaaaagcagagcaggcattgaggtaggacctgaagttgaggtaggacctgcacattgcatgggtcccggagcctctgttcatgttcaggatgtaattgtggcatcgagggatcctaaaaggaggtggcctctgagctgggccttggcaatggggtagaattgcagtaggcagagggagcagagatgggttttgtgcttgtaaacgggcattggtttactgtcactggtgggggtagcagctcttttgttctagctcttcatttccataatgcgtgttcttttttcatactttaggggaaagaaggaggagaactctattatttctatggggagaattcctcctaaacctgaagatcttaaaactaagcgaattattccctgttcattctccactgatgcagttcatgactgcaattgcaactgcctttcccgtcattttctatggccaaactcagtgtttttaagtgagctcttcttttaaaaaacaaaaacaaaaacaagtctcctgataaatccgtttgaaagacactaagttttgaaagttaagcaggctttgatcattcatggtacattgtgaattaccaaggagggagattgatatccttcatcgtacaatgcactcccctcctttttcttttgtatctggtggagtaagtcttccaagagcaattcttagagaaacaaagcctactctgtttccctgtttctagagttttccaacaagggtataaaaaagcctcagacagcttactattatccagtaacctcaggtacctcagtggctgcgtgttattccctttagaagtccaaaaactcactagcagagcaagaatatggatgtcaaagtgcagaactagaccctgacaaactagctctgtgcctgccacaaagcctcttaattaggaatgcagttcattgaataactgtatcaaagttagctggaataccttgacatgaagattccttccacttactgagcagtttgtgcccactagtggccagacacaggctttgctctagctaaaatacccccaggattacctgttgagagagccgcccacgtcaccttcatcgcctttgtgagctccatgctggcacatagttgtctccatctgttttgctttccgcatgaatcataggcaaagttagcctgaccagcaagcccatttcaaagccaccagctgggggagaagttgaagcccagggggcaagacccacgctgggctttgggcatgtttgagctggtgggcgaagcatatgggcaaaggccacattgtttaggatggaggcctttcagagactcagctatttctggaatgacattcatactgagaaataaggaaaatggcgatctgtgtgatggttgtgggttgggaggtttgggcgtgggagtcctggtcttggggtcatgtgttttgaaaacagtctagcactatgcaaatgggaggtgttaataactctttgcctctgtgatttaccctctcatggctttttctctcttggcctctccaaggtctcttctgactcttgcaactgtccttcctttcactttcaacaaaacctgttctcttgagtcagaacagttttatgaattgcccagagtgggacttgatatggggcagggttggttccatgctggtaatgaaagatgcacataaactgttacattgaaaagtgcttcatactttctgagcccttaactcactaggttgtaggccctgggctaagaatatagctgagggggctgatgtatttccttcattgctcagctgctcctacagcaggaatctgaccttgaaatgagctttgatctttgtgcaactcgagcttcccactatctgctgggggagctaggtctgggtgctgccacggcccatgtcagaaagctgtcagcagctcttataccggacactctcagaacaggaggcctgcaaagagctttagctttagttttgttgttgttgttgtttttgagacagtctcgctctgtcgcccaggctggagtgcagtggcgccatctcggctcactgcaagctccacctcccgggttcacaccattctcctgcctcagcctcccaagtagttgggactacaggtgcccgccaccacgcccagctaattttttgtatttttagtagagacgaggtttcaccgtgttagccaggatggtctcaatctcctgacctcgtgatccacccgccttggcctcccaaagtgctgggattacaggtgtgagccacggcgcccggccttttttttttttttttttttgagacgagtcttgctctgtttccaggctggagtgcagtggtgtgatctcggctcactgcagtctccgccttccgggttcaagtgattctcctgctccagcctcccaaatagctgggattacaggcacatgccaccacacccagctaatttttgtatttttagtagagacggggtttcaccatgttggccagtatgatctcgatctcctgaccttgtgatccacccgctttggcctcccaaagtgctgggattacaggcatgagccaccacgcccggccagagctttagtttttaattccagtgctcagcaccaaggttggatattggcttaaaacttccactggaaactccacaaatgtttagcaggcctccactttgcaccaggaactgagtgtggcctggaggacagcaggtactcaacataagttctactatttattgcatacttgcaaattgggtggggcgtggggaagagtatcgccacactggggcttgtgttaatctatagtttagccacttgggatgttggtgtcacactaggtttgatctaaatagaacaggtttgtaattgacagcattttcacttgcattatatttctctgaactcgaatcattattttctgtacagctgacttgttttgctgtagcagagtttggcagataatagcttgcatgtcgaatccaacctgcttttgtaaataagattttattggataaatggctattgaaggaatgggtcacacccactcatttacatcttgtccatggctatggctgattttgtgatacaaaggcagaattgagtagttgttaagaagaccgtatgacctgcaaagcctaaagtatttgctctctgattctttatagatagtttgctatctgtgcagatatttgcactctgactctgtagcacccttatgctggagaggagatatgaattgataccaaagttgtatcaaaacacttttggagttctggccaatgtgctaataacctggcttgtgtgaacattgtggctctcattttcttttttcttttttttttttttgagacgagtcttgctctgtcgcccaggctggagtgcagtggcgcagtggcgcgatctcggctcactgcaagctccgcctccagggttcacgccattctcctgcctcagcctccagagtagctgggactacaggcgcccgccaccacgcccggctaatttttttttttttgtatttttagtagagacgggttttcactgtgttagccagcatggtctcgatctcctgacctcgtgatctgccctcctgggcctcccaagtgctgggattacaggcgtgacccactgcgcccggcctgtggctcccattttcatactgcttggtgtgacccagatggaaatgctccagaacctggcccctcaataacagcctgtaaatgcttgtttaccctgttttctcacatcagccttttaggtggggttatttgcactatcctttaacagcgcttcaataacaactcttttgtttgatcattatgacaacccaataaaatatattagtgcctcaagtacgtaaagcagaattcaaggccaaaaaaatgtatgtggcaagttaggtgggcggtcagaagaacttggcaacagagcccagccctgcctgggctgtcctaaccagagcacataaatgctggagagagtcaggctttgcacgtgctcacacgggacccttaccttggacaggatcaaagccctgtcatttctggagtgtgtgatatctgctgtctcttactgcgctggttgttacctgtctattccagttctgatgaatggctctgattggggagcagcatcatctgagcttagaaatgtcctcagggccctgtggggttccctttgagcaacaacgagagctcttgaatcttgcaccagccttttggagctggaatggagtttgctttctcctgggctgggctctgttttttttcctctcgctgtccttgtgtagcttttgatcaatggcactggaggaaaggaaaatcccttgttgttttcctgggcatgtggaagcccagtcctgcccagaattccactagccctttgagttttggaatcctgactcttagtaagtcaatagagtgctgcatctgtttttacctttgtaatcttttaaattggttttaattatttaagtaatacaagtattataatacatgaaaatatcctcaactgaaaggcttcttgaccaaactcttcttcgttgcaacagtagttcctgggggaacccactgacatcaatttgacacaactttggctctttctctacgaatatttgcatgcctgtggatgtctatacgcaattacatagttttttggtgtttgttttcataatgatgacatgttgtgtgtgcaccttactcttttcattgtcttggagacctttcaaagactcttcattttctaactcatgatttttttaactgctgcaaaatatttcattgtgaggatataccataatttattttccctcatagaaatgctacaatcaatgtccttatttattttcctctgagtatatgttcactgtttttccaggatatagtagataagcacttgaacacatatagtgtcattacgctagaggtgttacaatttttaaactacttttaatggcaaaaattacaattacttttgtatcaacctaatatattctgccacaaaatggctggtcacaaaaggattataataataatattattattattcctgtgaatggtgtctgcaggtagctatcacttatttgttctgctgatattatccattttttttgaattttgcctattggctggatgaaaattggtgtcaccctcctcatgggtttctcttgcatttccttggttgctagtgaggttgggcaccttttacgatgtttattggctgttcatatttctcctttgctaatcacatggcttttcaattttatacttagctgttttctctttgttctgtctgtaatttaattttgttaatgatgcgaactagagatttaattttatgttttaccaaatgaatagccaacactatgtattgtctgttgtatttttccccactggtttatatgatgttgtgtattatctaccaaatttacatgtatgcttccaaagtcttattccatcccattcttttatttttctttcatgattaacaccatatttttgaattgctaaaactttataatataaatataagctttgtatgtctataaataaaatacattttattaattataattatatgttatataattataatataagcacttgaacatatatacttctataattatatattatataattatatatcatacttctatgatatgttctataattatatattgtatatatcatagttctatgatatgttctataattatatattataaagttacaattaattgtatatttaatttataccatatactgtaataagcaaaatatagaaatatgttttaaatctggtaggtggtatcttgactcattggttttatatgtttttctgattttctttttgttcatttgttcgttttggtttttagttgctgttgtgtttacttcaaattttttcttatctgtcattgctcattgttttttagatgaatttttaattgatttttttccagttccagttgcaatatggattaggacagcattgaatttgtagattattttggatgaaattggcatctttataactttgagtgtttctatccaggaacttagtatgtttctttagtacttcttttatgtacttgaataaaatttgtctaacttgttgatgtatccccaaagaaccagacatttttattaaatgtattttaatatttttttctgtctcattagtctgtgcttttatccatattaagtcaattaatttttggacttatttcattgttttttttctggcttcttgagttgaaagtgtagttcactctttttaaggatttttctagcaaatgcttagaagtgtatacattgtcctctgagaactgctttggttacaacccacaggtgtgccatgtggctctctatataattccttgctgaagaagtttttacttttcatcttgttttttttaatccgaggaattatctggaacactgtttttaaatttccaaatttattttggctagtttgtttttaatgtctaatttctgtttgcctttaggtatatgttgagcctacatcatttctgcttctatatttattgagatattctttgttgcccttatagtcagtttgaaaagctgtcccatgtgtgtttgaaaataactttgctatatagacacaagttttctatacatctattaggacagatttgttaatcctcttatgtaatctatattctttcttctttttggtctacctgacatctattttgaggaaggaagctaaagtcttctactgttattgtgattttaacaatttcttctcatattttgattactttttgattttaactttttcaggccatgttattatatgcataatgatttattttcttcagtcatagatcatgtaaaccagaagtaaagatcaaataaaccaaatctttcctctatgaaacaaccaaacaactctctggccacttttatcctttcaacataagttctattttttattattaatttatcttctttcttttttgttaacatttgcctgaagcttcttttcagaatcttttttgtttttgactgttgcttgttttaggtgtatctattatattagaatatagctacaatgtctctcttttttttttttttttttttttttgagacagagtctctctctgtgcccaggctggagtgcagtggtgtgatcttggctcactacaacctccacctcctgagttcaagcgattctctgcctcaacctcccaagtagctgggactataggtgtccaccagcacacctggccaatttttttgtatttttcgtagagatggggttttggcatgttggccaggctggtcttgaactcctggcctcaagtgatctacctgcctcagcctcccaaaatgtcttaatcaaagataaaattccatatcttatgagtgccttgaacccattcacatataatgtggttgatgatagacttactatcttaatgcatgtgttttatttattgtgcttatggttgaaaaccatcttcttcatctctggatcatcagagtcaaccagagggcttggctcaaagcagtaggtattcaacaaatgctcattgaataaaaatgtccttgatcctctaattctgatttgattcgagaaataacttatggcaaagagctcaattgtaatggggttgggactaaaattcaagttaatgttgaaagatgtttcaagaagtatttaggttatattattaaatattagacactttctgtttatatagatttaacatataaagattttaagttttctgtgtatgtgaggtctcgtagtagtttctgctgaaaatgacaggggcgcggggttaagggtgaagacagtatctcaggattggagaaggaaatctaagatattgtctccaccagccatactcctcatgaacatgcacatggaaggacatttcccagccaaagtgaatccctttatttgttttagtttcagatttgagtctcagatttctttgtaattccagctgcatctaccaaagtgctagataatttttttattttaagcaaacaatattatgagaacattatctttattgcaaagtgctcctccagaagaacctctatgctggataaagacaaacatttgattaaggttatgtaagagtgaaatcaggatggctcacaacccattagccgtcattttcttactttaattgacaaagaaattgaaaaaatgaaagaagcatatgttgtgcttgaatgtgaagagtgctagactagaatcaaaggcctcttagatttggctgtgtcatttgggctcatgaaaacacctctggaaatctctttcagtgcaaaggagctggactagttgatctctcaggtttcttccaactctaaaatatctccagtctgtgccttggaaacatcttaggtgaaaatctaggaacagttaacctaatttgcacccttaaaattctgccatgagctgcttacaactcaaaacaagtttatcttactcagttactaattataaaccatccagatttcagagctgtgagtactgggtgaagcattgaaggtatgcttttgaagccattacatatggcagttactgagctgaaaggattaaatgctgcagcttccccagttgcccttcctccatgagagcagtgcctgcccccagcattctgtggcacttggaagacaagacagaggccaaatgcagatttttaccctgggcttccctctacagtgtggaactcaggttgtttcttctttcctccctgaaatgacatgagtttgcagcggatggtgaactgaagaaaccataggaggctctgtcttcttgcctgaatttcagttggaagcttggagatttggggttcaacagagataaggaagtgtaagccttcatcccgtctggtggttggcgatcacacaccgctctgtgctgaggctaatggccatgatcagagttgaccaaaaaaaaaaaaaaaaaaaaatacgggttgtccaagcaaattgatttccatacctatagagagcatacctttcttcatcagtattttcttccattcttccaaaaaattactttgggctctaacagccattcccgtgatctttacctctccttggggaatgcagataatttgaatagtggttttaagctatttttcttggaatacagaagttctgataagcccttcaaagacccctgagggcaagaagagggaaggtagtaggcagggctcaggccctctttaacacagacacatgtacataagtaacacatttgcaccaacactggaaggaaattcaatatattttaaaatgactttaactcctagtgatggaactatagatatttttttacagcccttccctgaatgttcacatgtgttttttatatatatgtgtgtgtcacgtatttgtatatgtgtatatacacatatatatagaaacacgtacatatttgtatgcatatttgtacatataggtatatatgtatgtaatgtgtacatattggcacatgtgtgtgtttgtatatgtatatatcagtacatatagatgtatgtatatatgtatatattttttcctccaaaataaacaataaagaatgtcttcttagattgcaaaagtgaattgtctgagttctgctaaggaaaaaggacttctgcttctgctgcctgtgaggactgtgctgtcagcagtcatggcccttcaggccctgcacctcgaggcagagccctcccccggccatggccagctctcctaaccaagtgtctctttcccccagaatggctgcagtctgtccaggccaccatgatcctgtcgatcatcttcagcattctgtctctgttcctgttcttctgccaactcttcaccctcaccaaggggggcaggttttacatcactggaatcttccaaattcttgctggtaagttgtggatggtaaagtccatgtggaagcggggtgcatccaagtctgcggaatgattagtttagtagaaggatgtggcctcagaatgactgatgttcatgagtctccccactggatgctttccataaagtgagggtgggtgcttgtatgtgtgggtgtgtacctgtatgtgtcttagaacttgggacttagaactctccccttctccctggaatgagatgcatatgaaagagaacttagaggatctggaaggaaggtccccacccaagccaggcgtatcaacaggaatgaaactgcaatctggacacataatcagaggtgaatactgaggctatctgtagagcaaaggtcaggcttgagagctgtttctgtagattacattatgcctccagaaaatggccctgatgtgctaagaactagcaaagtagttatcaggtatgtgtcttccaccaataggtagtgatgaagccacactgacaaattctcaccttccttgcttccagttcctagattctactgggctttgattgactgttgtcatcctctggtgtcttcattttgacactcttgtgtcacatattgtcatttccaaacatggggctatgacaacacatgaaaacacatgagaggtctccttaatctcctgcctaaactgtcttcaagttccctctttaaatacgttattaatatgcatagtgtgcagagtcctagaaacttctgttacacagggtgacatcttccaactttgtctctggattctgcctagcatcttacatgcttacatcttacatcttacatcttacatcttgcattctgcctagcatcttacattagctcttacatgtctgtctgttgacttactgttgactgaaccagcagggcattggagagaagtaagagctagatgtagtggtggattctgtggtccaaattcatagatcacaaacttcatatgtaccagagtatgtctaggtactgggagatgttctcaattctgaccctctgagagggcaaaggatgtagcatctcttctctgagttggttgtcagaatgcccatggtaccatttcaccactctgtccccaggagcagtcattggaaggttgacgtaaatagggttgtatgggaagacacagcccaaggttagatgttggtgaccttgtctagaagacagagagttcccctttcctgaaaaaaggaagtaaatgattaaccacttctcattaaacactcaaatacaacatttcaatactcatggttttgagatttcaaaaccagacagtgctttgctacttacacatgtcttatgacaccaagccaagctcctggatggttgctggctctgttaaatgactaattatgcaaggagatatcatttctaggtacgttaaagtgaagagttacccttactcaattttcagttggaataaaaacaactgtaacatattctggggtttctttttttttttctcactcgttttagtttgatatcaaatcaaataatgatcatatccattgcatcagtggatatgccctcaagataatatggatttagaaccagaactttcataatgtatttctattgaaatgttagtttcataagcgatgattgggttttcatgcccatgtgtgagatgtgcctcgctcaaaccttgttatgatttggcacgttacccatctgatgtgaaaaaaattacattttatttgtacaggctcgttattttactgatgaataatttgagcccaccagaggataaatgaatgaccaaggtcacccagctcatgacagggacggttgagtgttacactgaatatagtgaggtacttcttatattttaaagacagaatgcaccaaaaaatttaaagaacacaaaatccaaggcagaagctctgccttttatattatcttttattggaactgatttacaatggaaggtaaatgcaaatttgcaccatgtattattctgaagttccaaacatctgtgatgaatacaagcctgtactataagacccagtcacattgaaaatatggagctgagaagaggtaagctgctgttgaatgggctccttgggatagccagtaccttcatcttcattcatcctgctgagctgtttcggctttaagttctttaacaatgtctttttagcaacatcattacatcattttaggccaaaactcaaagtccagagataagaacccttaagtcactcatgtaactgcactgtgtgttaaaagtatttcagttcagccaaacactcttctcctaggtattgcgatttaagtatatttactaatccactcttgcttcactattttcattctcctccaaagtcaatacaagatgtttagaactgtgctggaagtgcagaattccgaatgtaaaagcgcatgactttgtcctctttatcccctttacatctagctgcttacgtctcatgaaactgaattttcagttatctgttggtccacatttgaataagaaattatcttgaatttgaaatgctgagctgtaatagcagttgtaatttgtaagtcctgagagtgtcctgtcctccgttgttaatcccagtcaagatcatctgagagttggtctccagggaacctcagatctctaggatgttgcactggaaatggctgcaggatctttccactaattctgagaactgaaagagttaggaactctatttggagagttcctggttcccttatcgtgtgacagttctaagtcaattttgtcatgtggttttctgactcacagactagtaagaagttagtaattagagagctaagtagattagggttgttggagatgagaaacccacgttttgggaaaacctggcaagtgacaacttaacatcaaggaagtagtcagaaaagctaaaactgacaaacagaaggtagaagaaagtagctccacactcatgggatgtgaaatctacaagcgtgcatgcccggcaaatgcctctccaatgcactggagcgttttaagtggaaaccactagaatctctgtgtagtctccggaagtggctgtgagggctgcattatctctgcacagcttcctcttggtgggcccagctgtgatctttatggatggcacacatcagctttcaggaaaagcacatgaaaggtgctagggctcttggagctgactgtaggtttgggagttgtctgtctccttgctctagattacagctctgtgtgttgtgtggggtctccatggtttgccaaattatctcttcctcacttagccacaaggctgacagttaggaactatctcttcttgattgcattaggttggctgcttcctgaatgcatatcaaaaggctccttcctttagttcagtgctttcaacctgagctgtgcatcagaatcacctgaggtcctttaaaaaaaaaaaaaagacagtgggcggggcgcagtggctcacgcctgtaatcccagcactttgggaggctgaggtgggcagatcacttgaggtcaggagttcaagaccagcccggccaacatggtgaaaccctgtctctactaaaaataaaaataaaaaaatagccaggcgtggtggtgcgtgcctgtagtcccagctacttgggaaactgaggcagagaagaatcacttgaacctaggaggtggaggttgcagtgagccaagatcatgccactgcactccagcctgggggtgacagagtaagactgtctcaaaaaaaaagaaaaaagaaaagaaaaaagacagtattcaggtctcatccctggagactctcatttaatagttgtgggatggatccactgcctaggtgactctgatgtgcatttagggttgggaaccactgacatagccattaaactgtccctaatcccactgtaaggttttctaggatattttcccagaaataactaaaccaccttcttagagaaggaacatcctgatcctgcgtctggactttggagtcattcttattttcagaacctatagccatctattccttgacaagatctgttgggttgggttgcactagactggaaaacatcaagaaattaatctagacacagacctaaaaggaagatttgcacgttttgatttattttactgcttacacccagcatcaagctccatgtggggcacatagaggagcctgaataaaactattattggttggatgattagataattgtgtttcctcggcagaatcaaactcaagtcaaatgtgtggttaccgggattaagaacagaaagaatagaggggactcctgagtgagtttcactttctctctctttttgttattgttgttcatttgttttgttaatagaaagatcaatatagctatagagcatttaaaataaaaggatttgggccaggtgtggtggcgaatgcctgtaatcccaacactttgaaagactgaggtgagaggattgcttaaagccaggagttcaagaccagcctgggcaacaaagcgagatgccatctctacaaaaaatgatttaaaaattagtggggtgccttggagcacgcctgtggtcccagctattcaggaggctgaggcaggaggattgcttgaggctgagaggtcaagactacagtgaactatgatcaggcccctgcacttgctccagcctggtgacagagtgagaccctgcctctctaggaaaaaaaaaaaaagaatttggtggggggggattaataaaagttcagtgccatcctgttcagagtgattcaaaggtggcttaagtcaaataccacatttagaaatttttgttagggacggatgattatggatgtctcactggccatgcccaaatcagagtcaatgctatgggtggctttctaggcagcaatggtcatttggatactgaaaatgtaaaaggagtgggcttcaatcacagaaacacagagaagtctcttgctttcagaggaacaggcctcacagccccttcctgccctcccttgttgccctgaggatgaagtgggagggagaaaaaggcacccactcatcaacccaacatcatagctgcaccttccagtgaccaccctggtggtctcactgacatccctttccagccaacttctctctgacttctgccccaggctctcaaatgaaagtgcttttgcgaggttaccaaagtacactttgaagtctttaatttgttggatcactctgctcctttcaatactgttagctacttactctttgaagctctattcccctggcttctgagacatcgctgtgttcccacttgcctgagtgaacacaccaggcggtcatgctggcctctcctctctccctcaccccacagccagccttctactgaatcctgcatttgggtagcatcctccgtaggcattacgcccatttctttgtcttgactcactaaccccagtgcagctatgcaaaggcctcctcatgcaccttccagctacctgccttgtccctgcccctgaatccattttctgcaccatggccagagtgatcattctaaaacgtgcgtctgatcatgtttctcctctgctgaagtttcctcagaggcttctcatcacagcttccttaacatgtcatgtaaggccctctctttcctgacccttagtgagctaatcagcctattatatgagagtccgtgtggcttctccagttctccagtttggactcagcgcccgtctatgctcttggggcatctggtgcatttccttccctctctcgaaattccatcgaactatattgaaattatatgcaaatgtctgtcttctagacacacagtccttaaggtaggagccatgtctcacttatcttggtacatctagagcagcagctagaacagggcccgcatgcaataggcactcattaaatgttcactgaaccaaagggaatggagatgataaggatgtactgggaattccctcagctacttcctttgaccttggccccttgggtgcttccttccagaggctctgggctctaatcactctcatggggttgcagtcactgttaaaggagcttaagactttctcttcaaaagggctttaggcagaacttgcaggaagttgttgcagacctacccatcctgagacagggagaaactcttgtaagttgaatgctcagcacatttgtattgtctggacagggtcagtgtccttctgcttaaagatgacctatgctcccagactcaggcccctaccagggagtcccggtcattgccaaagagcagaacatctgcctgtgcctcagggcctcacttacccttgccacagggacctggtgatacatctccacctactggttgctgagaaagtgagtcacagtccacttactaggggtatttggcttttggagatgactactggatgacattaactgtcttgggttgcagacagagggaacccagccaaagaatcatccttttctctatttcagggtacatctattgctttacatgcagacaatcttgtaataatatattccctaaaacatcagtctcatgacaacaatcatataaagtgtgtatcctctctttggctttgtagatatctaagtttactgcttatcacggttaagcttagagtcattcacttctgagattctactaagatgaaagtcatacacattaagctgtgtagttagtcgctgcacttctctatactgcctctttctatccctcttgttcaaggatggaatctggtatctcctcttagtcagtgaaatcggggagtagctggtccactcagctcctgaggcatttctcatgccagatctggttagacagcccatttgggagccctgcctgcatgatgataagtgttgtcctgccctgctagcactctgttaacacctttctctctgccaaggtcatgtttccttttttatagccctgccttcattagataagtcagacagagcccaagagacaacttcagtatttcttaaagacaagagttagcactttggagatgcaagccgtggaagagtggggcaagataattaaggatcctggacctcaaggcaaataaagtgtgtccccagcagatcaaaactgctgttttctgcaatcaacaattaacccgcaatgatttcgctcctgacatggccatctttgctgtcttctgaacttcctgtaggtggcaggtcctggatgctgtcccattttggttccaagactattgtgtggctctcttgacaggggccagtcaagtggcagttttctcctctgagttgcaggaggacaggcttaacagggtggttcatgtggcgtattcccacttctgcactgtcttcaaacaaagttgcagtgtccagtaaaacagcagcccaaatagcagtgaggttagatgtaggtggccgggcggtgggggggcgcgggtgcattgggaactctggaactgcatgaatcccacacggcatactggatttataatagagtcactcagagcttctcaaagaccctgttttcagggaggaaacagaccaggctttgtgcagctccaggctggggtgctgacatcactggacccctccttgtggggggacgctgtctgtcacgtgagctggggccatcagtttgagcgtggctgctccacagggcccaagatggaggcttttcttctctttccaacaccgaagggctgcattggtgggccagggagggtggcctgacccacctgcatatccagtgccctttagccttctttgggcttcaaatgcaaccttggggcacccagggaaatggagcttgcctttgatcatcttccccctctgcagcgtggcttcccagtcatactgtcggctgcatccttattcctcactttctttttccctctccacacatttctttgtctgtttggcagtgcccataggcattgcttgcttcacctggtatgtggcaactctgttagggggaccataggcccctgtggtctgcatggagcttaatccttttaccatggcgtgactccaataatggtaaaatgtcacttcacttccattggctttaggtaatgccaccactcacttgtagagtggcatatagatttccaggggtttccatacccaaccacccttggaaagagacaggtaggtttttcttgcagcctgaatgcctgctggccatgtagtgtggcctgggaattggtgacagtgaaagaaagagaaatagggcagaaaatgtaaagaaaaccctcaggtattaaggcctaggtggccaagattggaaatagagagcaagttgttttccttgttcccatatttttgtgctacctcctttcaattctggacctggaagcagatttccttccagaggtagaaatgttcttcctacccagcaattgtcagcatccggggtggcggagagggggccgctctgccatggactctccgtcacccagcttgtccctctccttccccaggtctgtgcgtgatgagtgctgcggccatctacacggtgaggcacccggagtggcatctcaactcggattactcctacggtttcgcctacatcctggcctgggtggccttccccctggcccttctcagcggtgtcatctatgtgatcttgcggaaacgcgaatgaggcgcccagacggtctgtctgaggctctgagcgtacatagggaagggaggaagggaaaacagaaagcagacaaagaaaaaagagctagcccaaaatcccaaactcaaaccaaaccaaacagaaagcagtggaggtgggggttgctgttgattgaagatgtatataatatctccggtttataaaacctatttataacactttttacatatatgtacatagtattgtttgctttttatgttgaccatcagcctcgtgttgagccttaaagaagtagctaaggaactttacatcctaacagtataatccagctcagtatttttgttttgttttttgtttgtttgttttgttttacccagaaataagataactccatctcgccccttccctttcatctgaaagaagatacctccctcccagtccacctcatttagaaaaccaaagtgtgggtagaaaccccaaatgtccaaaagcccttttctggtgggtgacccagtgcatccaacagaaacagccgctgcccgaacctctgtgtgaagctttacgcgcacacggacaaaatgcccaaactggagcccttgcaaaaacacggcttgtggcattggcatacttgcccttacaggtggagtatcttcgtcacacatctaaatgagaaatcagtgacaacaagtctttgaaatggtgctatggatttaccattccttattatcactaatcatctaaacaactcactggaaatccaattaacaattttacaacataagatagaatggagacctgaataattctgtgtaatataaatggtttataactgcttttgtacctagctaggctgctattattactataatgagtaaatcataaagccttcatcactcccacatttttcttacggtcggagcatcagaacaagcgtctagactccttgggaccgtgagttcctagagcttggctgggtctaggctgttctgtgcctccaaggactgtctggcaatgacttgtattggccaccaactgtagatgtatatatggtgcccttctgatgctaagactccagaccttttgtttttgctttgcattttctgattttataccaactgtgtggactaagatgcattaaaataaacatcagagtaactcact

SEQ ID NO: 2 - RNA, 112 nt, synthetic construct
  source 1..112: mol_type = other RNA
gcagaaactccgctgagcagaacttgccgccagaatgctcctcctgttgctgagtatcatcgtcctccacgtcgcggtgctggtgctgctgttcgtctccacgatcgtcagc

SEQ ID NO: 3 - RNA, 100 nt, synthetic construct
  source 1..100: mol_type = other RNA
caatggatcgtgggcaatggacacgcaactgatctctggcagaactgtagcacctcttcctcaggaaatgtccaccactgtttctcatcatcaccaaacg

SEQ ID NO: 4 - RNA, 141 nt, synthetic construct
  source 1..141: mol_type = other RNA
aatggctgcagtctgtccaggccaccatgatcctgtcgatcatcttcagcattctgtctctgttcctgttcttctgccaactcttcaccctcaccaaggggggcaggttttacatcactggaatcttccaaattcttgctg

SEQ ID NO: 5 - RNA, 1302 nt, synthetic construct
  source 1..1302: mol_type = other RNA
gtctgtgcgtgatgagtgctgcggccatctacacggtgaggcacccggagtggcatctcaactcggattactcctacggtttcgcctacatcctggcctgggtggccttccccctggcccttctcagcggtgtcatctatgtgatcttgcggaaacgcgaatgaggcgcccagacggtctgtctgaggctctgagcgtacatagggaagggaggaagggaaaacagaaagcagacaaagaaaaaagagctagcccaaaatcccaaactcaaaccaaaccaaacagaaagcagtggaggtgggggttgctgttgattgaagatgtatataatatctccggtttataaaacctatttataacactttttacatatatgtacatagtattgtttgctttttatgttgaccatcagcctcgtgttgagccttaaagaagtagctaaggaactttacatcctaacagtataatccagctcagtatttttgttttgttttttgtttgtttgttttgttttacccagaaataagataactccatctcgccccttccctttcatctgaaagaagatacctccctcccagtccacctcatttagaaaaccaaagtgtgggtagaaaccccaaatgtccaaaagcccttttctggtgggtgacccagtgcatccaacagaaacagccgctgcccgaacctctgtgtgaagctttacgcgcacacggacaaaatgcccaaactggagcccttgcaaaaacacggcttgtggcattggcatacttgcccttacaggtggagtatcttcgtcacacatctaaatgagaaatcagtgacaacaagtctttgaaatggtgctatggatttaccattccttattatcactaatcatctaaacaactcactggaaatccaattaacaattttacaacataagatagaatggagacctgaataattctgtgtaatataaatggtttataactgcttttgtacctagctaggctgctattattactataatgagtaaatcataaagccttcatcactcccacatttttcttacggtcggagcatcagaacaagcgtctagactccttgggaccgtgagttcctagagcttggctgggtctaggctgttctgtgcctccaaggactgtctggcaatgacttgtattggccaccaactgtagatgtatatatggtgcccttctgatgctaagactccagaccttttgtttttgctttgcattttctgattttataccaactgtgtggactaagatgcattaaaataaacatcagagtaactca

SEQ ID NO: 6 - RNA, 25 nt, synthetic construct
  source 1..25: mol_type = other RNA
ctgcgaggagagcgctgggcgtgag

SEQ ID NO: 7 - RNA, 25 nt, synthetic construct
  source 1..25: mol_type = other RNA
aagttctgctcagcggagtttctgc

SEQ ID NO: 8 - RNA, 25 nt, synthetic construct
  source 1..25: mol_type = other RNA
caacaggaggagcattctggcggca

SEQ ID NO: 9 - RNA, 25 nt, synthetic construct
  source 1..25: mol_type = other RNA
ctcagcaacaggaggagcattctgg

SEQ ID NO: 10 - RNA, 25 nt, synthetic construct
  source 1..25: mol_type = other RNA
tgatactcagcaacaggaggagcat

SEQ ID NO: 11 - RNA, 20 nt, synthetic construct
  source 1..20: mol_type = other RNA
atactcagcaacaggaggag

SEQ ID NO: 12 - RNA, 20 nt, synthetic construct
  source 1..20: mol_type = other RNA
tgatactcagcaacaggagg

SEQ ID NO: 13 - RNA, 25 nt, synthetic construct
  source 1..25: mol_type = other RNA
gacgatgatactcagcaacaggagg

SEQ ID NO: 14 - RNA, 20 nt, synthetic construct
  source 1..20: mol_type = other RNA
gatgatactcagcaacagga

SEQ ID NO: 15 - RNA, 20 nt, synthetic construct
  source 1..20: mol_type = other RNA
acgatgatactcagcaacag

SEQ ID NO: 16 - RNA, 25 nt, synthetic construct
  source 1..25: mol_type = other RNA
tggaggacgatgatactcagcaaca

SEQ ID NO: 17 - RNA, 20 nt, synthetic construct
  source 1..20: mol_type = other RNA
ggacgatgatactcagcaac

SEQ ID NO: 18 - RNA, 20 nt, synthetic construct
  source 1..20: mol_type = other RNA
gaggacgatgatactcagca

SEQ ID NO: 19 - RNA, 20 nt, synthetic construct
  source 1..20: mol_type = other RNA
tggaggacgatgatactcag

SEQ ID NO: 20 - RNA, 25 nt, synthetic construct
  source 1..25: mol_type = other RNA
cgacgtggaggacgatgatactcag

SEQ ID NO: 21 - RNA, 20 nt, synthetic construct
  source 1..20: mol_type = other RNA
cgtggaggacgatgatactc

SEQ ID NO: 22 - RNA, 20 nt, synthetic construct
  source 1..20: mol_type = other RNA
gacgtggaggacgatgatac

SEQ ID NO: 23 - RNA, 25 nt, synthetic construct
  source 1..25: mol_type = other RNA
caccgcgacgtggaggacgatgata

SEQ ID NO: 24 - RNA, 20 nt, synthetic construct
  source 1..20: mol_type = other RNA
gcgacgtggaggacgatgat

SEQ ID NO: 25 - RNA, 25 nt, synthetic construct
  source 1..25: mol_type = other RNA
accagcaccgcgacgtggaggacga

SEQ ID NO: 26 - RNA, 25 nt, synthetic construct
  source 1..25: mol_type = other RNA
gcagcaccagcaccgcgacgtggag

SEQ ID NO: 27 - RNA, 25 nt, synthetic construct
  source 1..25: mol_type = other RNA
gaacagcagcaccagcaccgcgacg

SEQ ID NO: 28 - RNA, 25 nt, synthetic construct
  source 1..25: mol_type = other RNA
gagacgaacagcagcaccagcaccg

SEQ ID NO: 29 - RNA, 25 nt, synthetic construct
  source 1..25: mol_type = other RNA
aggcactcacgctgacgatcgtgga

SEQ ID NO: 30 - RNA, 25 nt, synthetic construct
  source 1..25: mol_type = other RNA
cgatccattgctagagagaatcaga

SEQ ID NO: 31 - RNA, 25 nt, synthetic construct
  source 1..25: mol_type = other RNA
cgtgtccattgcccacgatccattg

SEQ ID NO: 32 - RNA, 25 nt, synthetic construct
  source 1..25: mol_type = other RNA
ccagagatcagttgcgtgtccattg

SEQ ID NO: 33 - RNA, 25 nt, synthetic construct
  source 1..25: mol_type = other RNA
acagttctgccagagatcagttgcg

SEQ ID NO: 34 - RNA, 25 nt, synthetic construct
  source 1..25: mol_type = other RNA
gacatttcctgaggaagaggtgcta

SEQ ID NO: 35 - RNA, 25 nt, synthetic construct
  source 1..25: mol_type = other RNA
gatgagaaacagtggtggacatttc

SEQ ID NO: 36 - RNA, 25 nt, synthetic construct
  source 1..25: mol_type = other RNA
tttggtgatgatgagaaacagtggt

SEQ ID NO: 37 - RNA, 25 nt, synthetic construct
  source 1..25: mol_type = other RNA
agcctcaccgtttggtgatgatgag

SEQ ID NO: 38 - RNA, 25 nt, synthetic construct
  source 1..25: mol_type = other RNA
caccgtttggtgatgatgagaaaca

SEQ ID NO: 39 - RNA, 25 nt, synthetic construct
  source 1..25: mol_type = other RNA
cagactgcagccattctgggggaaa

SEQ ID NO: 40 - RNA, 25 nt, synthetic construct
  source 1..25: mol_type = other RNA
gaatgctgaagatgatcgacaggat

SEQ ID NO: 41 - RNA, 25 nt, synthetic construct
  source 1..25: mol_type = other RNA
agagttggcagaagaacaggaacag

SEQ ID NO: 42 - RNA, 25 nt, synthetic construct
  source 1..25: mol_type = other RNA
tgtaaaacctgccccccttggtgag

SEQ ID NO: 43 - RNA, 25 nt, synthetic construct
  source 1..25: mol_type = other RNA
attccagtgatgtaaaacctgcccc

SEQ ID NO: 44 - RNA, 25 nt, synthetic construct
  source 1..25: mol_type = other RNA
aatttggaagattccagtgatgtaa

SEQ ID NO: 45 - RNA, 25 nt, synthetic construct
  source 1..25: mol_type = other RNA
taccagcaagaatttggaagattcc

SEQ ID NO: 46 - RNA, 26 nt, synthetic construct
  source 1..26: mol_type = other RNA
cactcatcacgcacagacctggggaa

SEQ ID NO: 47 - RNA, 25 nt, synthetic construct
  source 1..25: mol_type = other RNA
gcctcaccgtgtagatggccgcagc

SEQ ID NO: 48 - RNA, 25 nt, synthetic construct
  source 1..25: mol_type = other RNA
ttgagatgccactccgggtgcctca

SEQ ID NO: 49 - RNA, 25 nt, synthetic construct
  source 1..25: mol_type = other RNA
ccgtaggagtaatccgagttgagat

SEQ ID NO: 50 - RNA, 25 nt, synthetic construct
  source 1..25: mol_type = other RNA
ctctgatgtttattttaatgcatct

SEQ ID NO: 51 - protein, 9 aa, synthetic construct
RRRQRRKKR

SEQ ID NO: 52 - protein, 9 aa, synthetic construct
RKKRRQRRR

SEQ ID NO: 53 - protein, 11 aa, synthetic construct
RRRRRRRRRFF

SEQ ID NO: 54 - protein, 11 aa, synthetic construct
RRRRRFFRRRR

SEQ ID NO: 55 - protein, 4 aa, synthetic construct
RRRR

SEQ ID NO: 56 - protein, 5 aa, synthetic construct
RRRRR

SEQ ID NO: 57 - protein, 6 aa, synthetic construct
RRRRRR

SEQ ID NO: 58 - protein, 7 aa, synthetic construct
RRRRRRR

SEQ ID NO: 59 - protein, 8 aa, synthetic construct
RRRRRRRR

SEQ ID NO: 60 - protein, 9 aa, synthetic construct
RRRRRRRRR

SEQ ID NO: 61 - protein, 12 aa, synthetic construct
  MOD_RES 2: note = 6-aminohexanoic acid
  MOD_RES 5: note = 6-aminohexanoic acid
  MOD_RES 8: note = 6-aminohexanoic acid
  MOD_RES 11: note = 6-aminohexanoic acid
RXRRXRRXRRXR

SEQ ID NO: 62 - protein, 15 aa, synthetic construct
  MOD_RES 2: note = 6-aminohexanoic acid
  MOD_RES 5: note = 6-aminohexanoic acid
  MOD_RES 8: note = 6-aminohexanoic acid
  MOD_RES 11: note = 6-aminohexanoic acid
  MOD_RES 14: note = 6-aminohexanoic acid
RXRRXRRXRRXRRXR

SEQ ID NO: 63 - protein, 12 aa, synthetic construct
  MOD_RES 2: note = 6-aminohexanoic acid
  SITE 5: note = bAla
  SITE 11: note = bAla
  MOD_RES 8: note = 6-aminohexanoic acid
RXRRXRRXRRXR

SEQ ID NO: 64 - protein, 14 aa, synthetic construct
RARRARRARRARFF

SEQ ID NO: 65 - protein, 14 aa, synthetic construct
RGRRGRRGRRGRFF

SEQ ID NO: 66 - DNA, 20 bp, synthetic construct
  source 1..20: mol_type = other DNA
ggaagaaggggttacgctgt

SEQ ID NO: 67 - DNA, 20 bp, synthetic construct
  source 1..20: mol_type = other DNA
ggaagaaggggttacgctgt

SEQ ID NO: 68 - DNA, 10 bp, synthetic construct
  source 1..10: mol_type = other DNA
gatcgtcagc

SEQ ID NO: 69 - DNA, 10 bp, synthetic construct
  source 1..10: mol_type = other DNA
gtgagtgcct

SEQ ID NO: 70 - DNA, 22 bp, synthetic construct
  source 1..22: mol_type = other DNA
tgtttctcatcatcaccaaacg

SEQ ID NO: 71 - skipped

SEQ ID NO: 72 - protein, 12 aa, synthetic construct
  MOD_RES 3: note = 6-aminohexanoic acid
  MOD_RES 6: note = 6-aminohexanoic acid
  MOD_RES 9: note = 6-aminohexanoic acid
  MOD_RES 12: note = 6-aminohexanoic acid
RRXRRXRRXRRX

(19) United States
(12) Patent Application Publication
(10) Pub. No.: US 2024/0318179 A1
(43) Pub. Date: Sep. 26, 2024

(54) MORPHOLINO OLIGOMERS FOR TREATMENT OF PERIPHERAL MYELIN PROTEIN 22 RELATED DISEASES

(71) Applicants: MURDOCH UNIVERSITY, Murdoch (AU); SAREPTA THERAPEUTICS, INC., Cambridge, MA (US)

(72) Inventors: Stephen Donald WILTON, Western Australia (AU); May Thandar AUNG-HTUT, Willetton, Western Australia (AU); Kevin KIM, Quincy, MA (US); Annika MALMBERG, Cambridge, MA (US); Kathy MORGAN, Cambridge, MA (US)

(21) Appl. No.: 18/703,047

(22) PCT Filed: Oct. 21, 2022
(86) PCT No.: PCT/US2022/078509

§ 371 Date: Apr. 19, 2024

Related U.S. Application Data
(60) Provisional application No. 63/262,907, filed on Oct. 22, 2021.
(60) Provisional application No. 63/377,066, filed on Sep. 26, 2022.

(51) Int. Cl. C12N 15/113

(52) U.S. Cl. CPC C12N 15/113; C12N 2310/11; C12N 2310/321; C12N 2310/3513

(57) ABSTRACT

Provided herein are antisense oligomers comprising a chemically modified antisense oligomer having a targeting sequence that is complementary to a target region of the human peripheral myelin protein 22 (PMP22) pre-mRNA. The antisense oligomer can be a peptide nucleic acid, a locked nucleic acid, phosphorodiamidate morpholino oligomer, a 2′-O-Me phosphorothioate oligomer, or a combination thereof. In an embodiment, the antisense oligomer is covalently linked to a cell-penetrating peptide. The antisense oligomers are useful for the treatment for various diseases in a subject in need thereof, including, but not limited to, a disease associated with dysregulation of peripheral myelin protein 22.

### RELATED APPLICATION

This application claims priority to U.S. Provisional Application No. 63/262,907, filed on Oct. 22, 2021, and U.S. Provisional Application No. 63/377,066, filed on Sep. 26, 2022. The entire contents of these applications are herein incorporated by reference.

### SEQUENCE LISTING

The instant application contains a Sequence Listing which has been submitted electronically in XML file format and is hereby incorporated by reference in its entirety. Said XML copy, created on Oct. 21, 2022, is named 732901_SPT-8399PC_SL.xml and is 105 KB in size.

### BACKGROUND OF THE INVENTION

Peripheral Myelin Protein 22 (PMP22) is a membrane protein that is encoded by the PMP22 gene in humans. Schwann cells show high expression of PMP22, wherein 2-5% of the total protein content in myelin is PMP22. Thus, PMP22 plays an essential role in the formation and maintenance of compact myelin.

Alterations of PMP22 gene expression are associated with a variety of neuropathies, such as Charcot-Marie-Tooth type 1A (CMT1A). CMT1A is typically caused by overexpression or duplication of the PMP22 gene. CMT1A affects about 6.9 out of every 100,000 people. Progression of this disease is characterized by loss of muscle tissue and touch sensation across the body. Currently, there are no curative treatments for CMT1A.

There remains a need for therapeutic molecules effective for the treatment of CMT1A.

### SUMMARY OF THE INVENTION

Provided herein are antisense oligomers comprising a chemically modified antisense oligomer having a targeting sequence that is complementary to a target region of the human peripheral myelin protein 22 (PMP22) pre-mRNA. The antisense oligomer can be any modified antisense oligomer, for example a peptide nucleic acid, a locked nucleic acid, phosphorodiamidate morpholino oligomer, a 2′-O-Me phosphorothioate oligomer, or a combination thereof. In an embodiment, the antisense oligomer is covalently linked to a cell-penetrating peptide. The antisense oligomers are useful for the treatment of a disease associated with dysregulation of peripheral myelin protein 22 in a subject in need thereof.

In certain embodiments, the antisense oligomer induces skipping of one or more of exon 2 (SEQ ID NO: 2), exon 3 (SEQ ID NO: 3), exon 4 (SEQ ID NO: 4), or exon 5 (SEQ ID NO: 5) of the PMP22 pre-mRNA.

In certain embodiments, the targeting sequence is complementary to a region within one of exon 2 (SEQ ID NO: 2), exon 3 (SEQ ID NO: 3), exon 4 (SEQ ID NO: 4), or exon 5 (SEQ ID NO: 5). The targeting sequence can also be complementary to a region spanning an exon/intron junction of exon 2 (SEQ ID NO: 2), exon 3 (SEQ ID NO: 3), exon 4 (SEQ ID NO: 4), or exon 5 (SEQ ID NO: 5).

In an embodiment, the antisense oligomer is a peptide-oligonucleotide conjugate of Formula I:

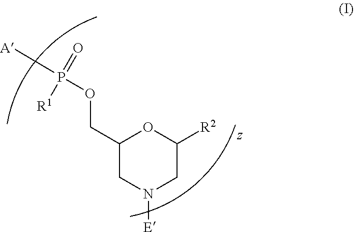

or a pharmaceutically acceptable salt thereof, wherein A′, E′, R¹, R², and z are as defined herein.

In certain embodiments, the antisense oligomer of Formula I is a peptide-oligonucleotide conjugate selected from:

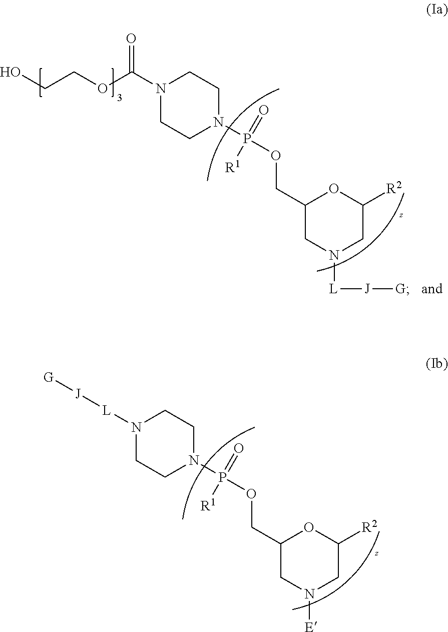

wherein A′, E′, G, J, L, R¹, R², and z are as defined herein.

In certain embodiments, each R¹ is N(CH₃)₂. In certain embodiments, each R₂ is independently selected from a naturally or non-naturally occurring nucleobase and the sequence formed by the combination of each R² from 5′ to 3′ is a targeting sequence. In certain embodiments, the cell-penetrating peptide is selected from rTAT, Tat, R₉F₂, R₅F₂R₄, R₄, R₅, R₆, R₇, R₈, R₉, (RAhxR)₄, (RAhxR)₅, (RAhxRRBR)₂, (RAR)₄F₂, and (RGR)₄F₂.

In another aspect, provided herein is a pharmaceutical composition comprising an antisense oligomer provided herein and a pharmaceutically acceptable carrier.

Also provided herein is a method of treating a disease associated with dysregulation of peripheral myelin protein 22 comprising administering to a subject in need thereof an antisense oligomer provided herein.

In another aspect, provided herein is the use of any of the antisense oligomers provided herein for treating a disease associated with dysregulation of peripheral myelin protein 22 in a subject in need thereof.

In yet another aspect, provided herein is an antisense oligomer provided herein for use in the manufacture of a medicament for treatment of a disease associated with dysregulation of peripheral myelin protein 22.

### DETAILED DESCRIPTION OF THE INVENTION

Many oligonucleotide analogs have been developed in which the phosphodiester linkages of native DNA are replaced by other linkages that are resistant to nuclease degradation. See, e.g., Barawkar and Bruice (1998) Proc Natl Acad Sci USA 95(1): 11047-52; Linkletter et al. (2001) Nucleic Acids Res 29(11):2370-6; and Micklefield (2001) Curr Med Chem 8(10): 1157-79. Antisense oligonucleotides having various backbone modifications other than to the internucleoside linkage have also been prepared (Crooke (2001) Antisense Drug Technology: Principles, Strategies, and Applications. New York, Marcel Dekker; Micklefield (2001)). In addition, oligonucleotides have been modified by peptide conjugation in order to enhance cellular uptake. See, e.g., Moulton et al. (2004) Bioconjug Chem 15(2): 290-9; and Nelson et al. (2005) Bioconjug Chem 16(4):959-66.

Morpholino-based oligomers (including antisense oligomers) are detailed, for example, in U.S. Pat. Nos. 5,698,685; 5,217,866; 5,142,047; 5,034,506; 5,166,315; 5,185,444; 5,521,063; 5,506,337; and PCT Publication Nos. WO/2009/064471, WO/2012/043730, WO 2008/036127; and Summerton et al. 1997, Antisense and Nucleic Acid Drug Development, 7, 187-195; all of which are hereby incorporated by reference in their entirety.

Provided herein are antisense oligomers comprising a chemically modified antisense oligomer having a targeting sequence that is complementary to a target region of the human peripheral myelin protein 22 (PMP22) pre-mRNA. The antisense oligomer can be any modified antisense oligomer, for example a peptide nucleic acid, a locked nucleic acid, phosphorodiamidate morpholino oligomer, a 2′-O-Me phosphorothioate oligomer, or a combination thereof. In an embodiment, the antisense oligomer is covalently linked to a cell-penetrating peptide. The antisense oligomers are useful for the treatment for various diseases in a subject in need thereof, including, but not limited to, Charcot-Marie-Tooth type 1A (CMT1A).

### I. Definitions

Unless defined otherwise, all technical and scientific terms used herein have the same meaning as commonly understood by those of ordinary skill in the art to which the disclosure belongs. Although any methods and materials similar or equivalent to those described herein can be used in the practice or testing of the subject matter of the present disclosure, preferred methods and materials are described. For the purposes of the present disclosure, the following terms are defined below.

The term “about” will be understood by persons of ordinary skill in the art and will vary to some extent on the context in which it is used. As used herein when referring to a measurable value such as an amount, a temporal duration, and the like, the term “about” is meant to encompass variations of ±10%, including ±5%, ±1%, and ±0.1% from the specified value, as such variations are appropriate to perform the disclosed methods.

The term “alkyl” refers to saturated, straight- or branched-chain hydrocarbon moieties containing, in certain embodiments, between one and six, or one and eight carbon atoms, respectively. Examples of C₁₋₆-alkyl moieties include, but are not limited to, methyl, ethyl, propyl, isopropyl, n-butyl, tert-butyl, neopentyl, n-hexyl moieties; and examples of C₁₋₈-alkyl moieties include, but are not limited to, methyl, ethyl, propyl, isopropyl, n-butyl, tert-butyl, neopentyl, n-hexyl, heptyl, and octyl moieties.

The number of carbon atoms in an alkyl substituent can be indicated by the prefix “C_{x-y},” where x is the minimum and y is the maximum number of carbon atoms in the substituent. Likewise, a Cₓ chain means an alkyl chain containing x carbon atoms.

The term “heteroalkyl” by itself or in combination with another term means, unless otherwise stated, a stable straight or branched chain alkyl group consisting of the stated number of carbon atoms and one or two heteroatoms selected from the group consisting of O, N, and S, and wherein the nitrogen and sulfur atoms may be optionally oxidized and the nitrogen heteroatom may be optionally quaternized. The heteroatom(s) may be placed at any position of the heteroalkyl group, including between the rest of the heteroalkyl group and the fragment to which it is attached, as well as attached to the most distal carbon atom in the heteroalkyl group. Examples include: —O—CH₂—CH₂—CH₃, —CH₂—CH₂—CH₂—OH, —CH₂—CH₂—NH—CH₃, —CH₂—S—CH₂—CH₃, and —CH₂—CH₂—S(═O)—CH₃. Up to two heteroatoms may be consecutive, such as, for example, —CH₂—NH—OCH₃, or—CH₂—CH₂—S—S—CH₃.

The term “aryl,” employed alone or in combination with other terms, means, unless otherwise stated, a carbocyclic aromatic system containing one or more rings (typically one, two, or three rings), wherein such rings may be attached together in a pendent manner, such as a biphenyl, or may be fused, such as naphthalene. Examples of aryl groups include phenyl, anthracyl, and naphthyl. In various embodiments, examples of an aryl group may include phenyl (e.g., C₆-aryl) and biphenyl (e.g., C₁₂-aryl). In some embodiments, aryl groups have from six to sixteen carbon atoms. In some embodiments, aryl groups have from six to twelve carbon atoms (e.g., C₆₋₁₂-aryl). In some embodiments, aryl groups have six carbon atoms (e.g., C₆-aryl).

As used herein, the term “heteroaryl” or “heteroaromatic” refers to a heterocycle having aromatic character. Heteroaryl substituents may be defined by the number of carbon atoms, e.g., C₁₋₉-heteroaryl indicates the number of carbon atoms contained in the heteroaryl group without including the number of heteroatoms. For example, a C₁₋₉-heteroaryl will include an additional one to four heteroatoms. A polycyclic heteroaryl may include one or more rings that are partially saturated. Non-limiting examples of heteroaryls include pyridyl, pyrazinyl, pyrimidinyl (including, e.g., 2- and 4-pyrimidinyl), pyridazinyl, thienyl, furyl, pyrrolyl (including, e.g., 2-pyrrolyl), imidazolyl, thiazolyl, oxazolyl, pyrazolyl (including, e.g., 3- and 5-pyrazolyl), isothiazolyl, 1,2,3-triazolyl, 1,2,4-triazolyl, 1,3,4-triazolyl, tetrazolyl, 1,2,3-thiadiazolyl, 1,2,3-oxadiazolyl, 1,3,4-thiadiazolyl and 1,3,4-oxadiazolyl.

Non-limiting examples of polycyclic heterocycles and heteroaryls include indolyl (including, e.g., 3-, 4-, 5-, 6- and 7-indolyl), indolinyl, quinolyl, tetrahydroquinolyl, isoquinolyl (including, e.g., 1- and 5-isoquinolyl), 1,2,3,4-tetrahydroisoquinolyl, cinnolinyl, quinoxalinyl (including, e.g., 2- and 5-quinoxalinyl), quinazolinyl, phthalazinyl, 1,8-naphthyridinyl, 1,4-benzodioxanyl, coumarin, dihydrocoumarin, 1,5-naphthyridinyl, benzofuryl (including, e.g., 3-, 4-, 5-, 6- and 7-benzofuryl), 2,3-dihydrobenzofuryl, 1,2-benzisoxazolyl, benzothienyl (including, e.g., 3-, 4-, 5-, 6-, and 7-benzothienyl), benzoxazolyl, benzothiazolyl (including, e.g., 2-benzothiazolyl and 5-benzothiazolyl), purinyl, benzimidazolyl (including, e.g., 2-benzimidazolyl), benzotriazolyl, thioxanthinyl, carbazolyl, carbolinyl, acridinyl, pyrrolizidinyl, and quinolizidinyl.

As used herein, the acronym DBCO refers to 8,9-dihydro-3H-dibenzo[b,f][1,2,3]triazolo[4,5-d]azocine.

The term “protecting group” or “chemical protecting group” refers to chemical moieties that block some or all reactive moieties of a compound and prevent such moieties from participating in chemical reactions until the protective group is removed, for example, those moieties listed and described in T. W. Greene, P. G. M. Wuts, Protective Groups in Organic Synthesis, 3rd ed. John Wiley & Sons (1999). It may be advantageous, where different protecting groups are employed, that each (different) protective group be removable by a different means. Protective groups that are cleaved under totally disparate reaction conditions allow differential removal of such protecting groups. For example, protective groups can be removed by acid, base, and hydrogenolysis. Groups such as trityl, monomethoxytrityl, dimethoxytrityl, acetal and tert-butyldimethylsilyl are acid labile and may be used to protect carboxy and hydroxy reactive moieties in the presence of amino groups protected with Cbz groups, which are removable by hydrogenolysis, and Fmoc groups, which are base labile. Carboxylic acid moieties may be blocked with base labile groups such as, without limitation, methyl, or ethyl, and hydroxy reactive moieties may be blocked with base labile groups such as acetyl in the presence of amines blocked with acid labile groups such as tert-butyl carbamate or with carbamates that are both acid and base stable but hydrolytically removable.

Carboxylic acid and hydroxyl reactive moieties may also be blocked with hydrolytically removable protective groups such as the benzyl group, while amine groups may be blocked with base labile groups such as Fmoc. A particularly useful amine protecting group for the synthesis of compounds of Formula (I) is the trifluoroacetamide. Carboxylic acid reactive moieties may be blocked with oxidatively-removable protective groups such as 2,4-dimethoxybenzyl, while coexisting amino groups may be blocked with fluoride labile silyl carbamates.

Allyl blocking groups are useful in the presence of acid- and base-protecting groups since the former are stable and can be subsequently removed by metal or pi-acid catalysts. For example, an allyl-blocked carboxylic acid can be deprotected with a palladium(0)-catalyzed reaction in the presence of acid labile t-butyl carbamate or base-labile acetate amine protecting groups. Yet another form of protecting group is a resin to which a compound or intermediate may be attached. As long as the residue is attached to the resin, that functional group is blocked and cannot react. Once released from the resin, the functional group is available to react.

The term “nucleobase,” “base pairing moiety,” “nucleobase-pairing moiety,” or “base” refers to the heterocyclic ring portion of a nucleoside, nucleotide, and/or morpholino subunit. Nucleobases may be naturally occurring, or may be modified or analogs of these naturally occurring nucleobases, e.g., one or more nitrogen atoms of the nucleobase may be independently at each occurrence replaced by carbon. Exemplary analogs include hypoxanthine (the base component of the nucleoside inosine); 2, 6-diaminopurine; 5-methyl cytosine; C₅-propynyl-modified pyrimidines; 10-(9-(aminoethoxy)phenoxazinyl) (G-clamp) and the like.

Further examples of base pairing moieties include, but are not limited to, uracil, thymine, adenine, cytosine, guanine and hypoxanthine having their respective amino groups protected by acyl protecting groups, 2-fluorouracil, 2-fluorocytosine, 5-bromouracil, 5-iodouracil, 2,6-diaminopurine, azacytosine, pyrimidine analogs such as pseudoisocytosine and pseudouracil and other modified nucleobases such as 8-substituted purines, xanthine, or hypoxanthine (the latter two being the natural degradation products). The modified nucleobases disclosed in Chiu and Rana (2003) RNA 9:1034-1048, Limbach et al. (1994) Nucleic Acids Res. 22:2183-2196 and Revankar and Rao, Comprehensive Natural Products Chemistry, vol. 7, 313, are also contemplated, the contents of which are incorporated herein by reference.

Further examples of base pairing moieties include, but are not limited to, expanded-size nucleobases in which one or more benzene rings has been added. Nucleic base replacements described in the Glen Research catalog (www.glenresearch.com); Krueger A T et al. (2007) Acc. Chem. Res. 40:141-150; Kool E T (2002) Acc. Chem. Res. 35:936-943; Benner S A et al. (2005) Nat. Rev. Genet. 6:553-543; Romesberg F E et al. (2003) Curr. Opin. Chem. Biol. 7:723-733; Hirao, I (2006) Curr. Opin. Chem. Biol. 10:622-627, the contents of which are incorporated herein by reference, are contemplated as useful for the synthesis of the oligomers described herein. Examples of expanded-size nucleobases are shown below:

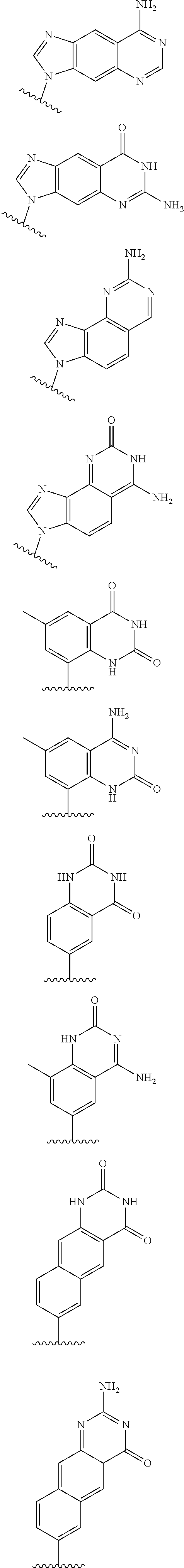

The terms “oligonucleotide” or “oligomer” refer to a compound comprising a plurality of linked nucleosides, nucleotides, or a combination of both nucleosides and nucleotides. In specific embodiments provided herein, an oligonucleotide is a morpholino oligonucleotide.

The phrase “morpholino oligonucleotide” or “PMO” refers to a modified oligonucleotide having morpholino subunits linked together by phosphoramidate or phosphorodiamidate linkages, joining the morpholino nitrogen of one subunit to the 5′-exocyclic carbon of an adjacent subunit. Each morpholino subunit comprises a nucleobase-pairing moiety effective to bind, by nucleobase-specific hydrogen bonding, to a nucleobase in a target.

As used herein, the terms “antisense oligomer” or “antisense compound” are used interchangeably and refer to a sequence of subunits, each having a base carried on a backbone subunit composed of ribose or other pentose sugar or morpholino group, and where the backbone groups are linked by intersubunit linkages that allow the bases in the compound to hybridize to a target sequence in a nucleic acid (typically an RNA) by Watson-Crick base pairing, to form a nucleic acid:oligomer heteroduplex within the target sequence. The oligomer may have exact sequence complementarity to the target sequence or nearly exact complementarity. Such antisense oligomers are designed to block or inhibit translation of the mRNA containing the target sequence, and may be said to be “directed to” a sequence with which it hybridizes.

Also contemplated herein as types of “antisense oligomer” or “antisense compound” are phosphorothioate-modified oligomers, peptide nucleic acids (PNAs), locked nucleic acids (LNAs), 2′-fluoro-modified oligomers, 2′-O,4′-C-ethylene-bridged nucleic acids (ENAs), tricyclo-DNAs, tricylo-DNA phosphorothioate-modified oligomers, 2′-O-[2-(N-methylcarbamoyl) ethyl] modified oligomers, 2′-O-methyl phosphorothioate modified oligomers, 2′-O-methoxyethyl (2′-O-MOE) modified oligomers, and 2′-O-Methyl oligonucleotides, or combinations thereof, as well as other antisense agents known in the art.

An antisense oligomer “specifically hybridizes” to a target polynucleotide if the oligomer hybridizes to the target under physiological conditions, with a Tm greater than 37° C., greater than 45° C., preferably at least 50° C., and typically 60° C.-80° C. or higher. The “Tm” of an oligomer is the temperature at which 50% hybridizes to a complementary polynucleotide. Tm is determined under standard conditions in physiological saline, as described, for example, in Miyada et al. (1987) Methods Enzymol. 154:94-107. Such hybridization may occur with “near” or “substantial” complementarity of the antisense oligomer to the target sequence, as well as with exact complementarity.

As used herein, the term “exon/intron gap junction” or “exon/intron junction” refers to the nucleic acid sequence region that corresponds to the 3′ end of an exon or intron and the 5′ end of the intron or exon that immediately proceeds said exon or intron. By way of example, but in no way limiting, the exon/intron junction of the sequence GATCGTCAGC (SEQ ID NO: 68)|GTGAGTGCCT (SEQ ID NO: 69) is represented by “|”. As such, GATCGTCAGC (SEQ ID NO: 68) corresponds to the last ten nucleotides of the 3′ end of an exon, and GTGAGTGCCT (SEQ ID NO: 69) corresponds to the first 10 nucleotides of the 5′ end of the proceeding intron. Similarly, the exon/intron junction of the sequence TGTTTCTCATCATCACCAAACG (SEQ ID NO: 70)|GTG (SEQ ID NO: 71) is represented by “I”. The antisense oligomer CACCGTTTGGTGATGATGAGAAACA (SEQ ID NO: 38) is complementary to the exon/intron junction TGTTTCTCATCATCACCAAACG (SEQ ID NO: 70)|GTG (SEQ ID NO: 71). An antisense oligomer with a targeting sequence that is complementary to a region spanning an exon/intron junction will have at least one nucleotide with complementarity to an exon and at least one nucleotide with complementarity to an intron.

The terms “complementary” and “complementarity” refer to oligonucleotides (i.e., a sequence of nucleotides) related by base-pairing rules. For example, the sequence “T-G-A (5′-3′)” is complementary to the sequence “T-C-A (5′-3′).” Complementarity may be “partial,” in which only some of the nucleic acids' bases are matched according to base pairing rules. Or, there may be “complete,” “total,” or “perfect” (100%) complementarity between the nucleic acids. The degree of complementarity between nucleic acid strands has significant effects on the efficiency and strength of hybridization between nucleic acid strands. While perfect complementarity is often desired, some embodiments can include one or more but preferably 6, 5, 4, 3, 2, or 1 mismatches with respect to the target RNA. Such hybridization may occur with “near” or “substantial” complementarity of the antisense oligomer to the target sequence, as well as with exact complementarity. In some embodiments, an oligomer may hybridize to a target sequence at about 50%, 55%, 60%, 65%, 70%, 75%, 80%, 85%, 90%, 95%, 99% or 100% complementarity. Variations at any location within the oligomer are included. In certain embodiments, variations in sequence near the termini of an oligomer are generally preferable to variations in the interior, and if present are typically within about 6, 5, 4, 3, 2, or 1 nucleotides of the 5′-terminus, 3′-terminus, or both termini.

Naturally occurring nucleotide bases include adenine, guanine, cytosine, thymine, and uracil, which have the symbols A, G, C, T, and U, respectively. Nucleotide bases can also encompass analogs of naturally occurring nucleotide bases. Base pairing typically occurs between purine A and pyrimidine T or U, and between purine G and pyrimidine C.

Oligonucleotides may also include nucleobase (often referred to in the art simply as “base”) modifications or substitutions. Oligonucleotides containing a modified or substituted base include oligonucleotides in which one or more purine or pyrimidine bases most commonly found in nucleic acids are replaced with less common or non-natural bases. In some embodiments, the nucleobase is covalently linked at the N9 atom of the purine base, or at the N1 atom of the pyrimidine base, to the morpholine ring of a nucleotide or nucleoside.

Purine bases comprise a pyrimidine ring fused to an imidazole ring, as described by the general formula:

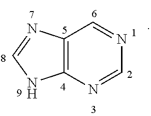

Adenine and guanine are the two purine nucleobases most commonly found in nucleic acids. These may be substituted with other naturally-occurring purines, including but not limited to N6-methyladenine, N2-methylguanine, hypoxanthine, and 7-methylguanine.

Pyrimidine bases comprise a six-membered pyrimidine ring as described by the general formula:

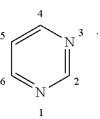

Cytosine, uracil, and thymine are the pyrimidine bases most commonly found in nucleic acids. These may be substituted with other naturally-occurring pyrimidines, including but not limited to 5-methylcytosine, 5-hydroxymethylcytosine, pseudouracil, and 4-thiouracil. In one embodiment, the oligonucleotides described herein contain thymine bases in place of uracil.

Other modified or substituted bases include, but are not limited to, 2,6-diaminopurine, orotic acid, agmatidine, lysidine, 2-thiopyrimidine (e.g. 2-thiouracil, 2-thiothymine), G-clamp and its derivatives, 5-substituted pyrimidine (e.g. 5-halouracil, 5-propynyluracil, 5-propynylcytosine, 5-aminomethyluracil, 5-hydroxymethyluracil, 5-aminomethylcytosine, 5-hydroxymethylcytosine, Super T), 7-deazaguanine, 7-deazaadenine, 7-aza-2,6-diaminopurine, 8-aza-7-deazaguanine, 8-aza-7-deazaadenine, 8-aza-7-deaza-2,6-diaminopurine, Super G, Super A, and N4-ethylcytosine, or derivatives thereof; N2-cyclopentylguanine (cPent-G), N2-cyclopentyl-2-aminopurine (cPent-AP), and N2-propyl-2-aminopurine (Pr-AP), pseudouracil or derivatives thereof; and degenerate or universal bases, like 2,6-difluorotoluene or absent bases like abasic sites (e.g. 1-deoxyribose, 1,2-dideoxyribose, I-deoxy-2-O-methylribose; or pyrrolidine derivatives in which the ring oxygen has been replaced with nitrogen (azaribose)). Pseudouracil is a naturally occurring isomerized version of uracil, with a C-glycoside rather than the regular N-glycoside as in uridine.

Certain modified or substituted nucleobases are particularly useful for increasing the binding affinity of the antisense oligonucleotides of the disclosure. These include 5-substituted pyrimidines, 6-azapyrimidines and N-2, N-6 and 0-6 substituted purines, including 2-aminopropyladenine, 5-propynyluracil and 5-propynylcytosine. In various embodiments, nucleobases may include 5-methylcytosine substitutions, which have been shown to increase nucleic acid duplex stability by 0.6-1.2° C.

In some embodiments, modified or substituted nucleobases are useful for facilitating purification of antisense oligonucleotides. For example, in certain embodiments, antisense oligonucleotides may contain three or more (e.g., 3, 4, 5, 6 or more) consecutive guanine bases. In certain antisense oligonucleotides, a string of three or more consecutive guanine bases can result in aggregation of the oligonucleotides, complicating purification. In such antisense oligonucleotides, one or more of the consecutive guanines can be substituted with hypoxanthine. The substitution of hypoxanthine for one or more guanines in a string of three or more consecutive guanine bases can reduce aggregation of the antisense oligonucleotide, thereby facilitating purification.

The oligonucleotides provided herein are synthesized and do not include antisense compositions of biological origin. The molecules of the disclosure may also be mixed, encapsulated, conjugated or otherwise associated with other molecules, molecule structures or mixtures of compounds, as for example, liposomes, receptor targeted molecules, oral, rectal, topical or other formulations, for assisting in uptake, distribution, or absorption, or a combination thereof.

As used herein, a “nucleic acid analog” refers to a non-naturally occurring nucleic acid molecule. A nucleic acid is a polymer of nucleotide subunits linked together into a linear structure. Each nucleotide consists of a nitrogen-containing aromatic base attached to a pentose (five-carbon) sugar, which is in turn attached to a phosphate group. Successive phosphate groups are linked together through phosphodiester bonds to form the polymer. The two common forms of naturally occurring nucleic acids are deoxyribonucleic acid (DNA) and ribonucleic acid (RNA). One end of the chain carries a free phosphate group attached to the 5′-carbon atom of a sugar moiety; this is called the 5′ end of the molecule. The other end has a free hydroxyl (—OH) group at the 3′-carbon of a sugar moiety and is called the 3′ end of the molecule. A nucleic acid analog can include one or more non-naturally occurring nucleobases, sugars, and/or internucleotide linkages, for example, a phosphorodiamidate morpholino oligomer (PMO). As disclosed herein, in certain embodiments, a “nucleic acid analog” is a PMO, and in certain embodiments, a “nucleic acid analog” is a positively charged cationic PMO.

A “morpholino oligomer” or “PMO” refers to a polymeric molecule having a backbone that supports bases capable of hydrogen bonding to typical polynucleotides, wherein the polymer lacks a pentose sugar backbone moiety, and more specifically a ribose backbone linked by phosphodiester bonds which is typical of nucleotides and nucleosides, but instead contains a ring nitrogen with coupling through the ring nitrogen. An exemplary “morpholino” oligomer comprises morpholino subunit structures linked together by phosphoramidate or phosphorodiamidate linkages, joining the morpholino nitrogen of one subunit to the 5′ exocyclic carbon of an adjacent subunit, each subunit comprising a purine or pyrimidine base-pairing moiety effective to bind, by base-specific hydrogen bonding, to a base in a polynucleotide. Morpholino oligomers (including antisense oligomers) are detailed, for example, in U.S. Pat. Nos. 5,034,506; 5, 142,047; 5, 166,315; 5, 185,444; 5,217,866; 5,506,337; 5,521,063; 5,698,685; 8,076,476; and 8,299,206; and PCT publication number WO 2009/064471, all of which are incorporated herein by reference in their entirety.

A preferred morpholino oligomer is a phosphorodiamidate-linked morpholino oligomer, referred to herein as a PMO. Such oligomers are composed of morpholino subunit structures such as shown below:

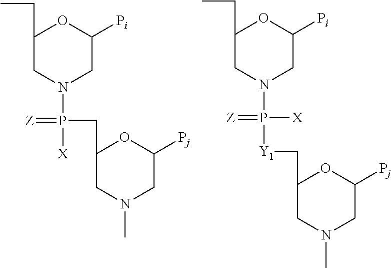

where X is NH₂, NHR, or NR₂ (where R is lower alkyl, preferably methyl), Y₁ is O, and Z is O, and Pᵢ and Pⱼ are purine or pyrimidine base-pairing moieties effective to bind, by base-specific hydrogen bonding, to a base in a polynucleotide. Also preferred are structures having an alternate phosphorodiamidate linkage, where X is lower alkoxy, such as methoxy or ethoxy, Y₁ is NH or NR, where R is lower alkyl, and Z is O.

Representative PMOs include PMOs wherein the intersubunit linkages are linkage (A1). See Table 1.

TABLE 1
Representative Intersubunit Linkages

| No. | Name | Structure |
|---|---|---|
| A1 | PMO | 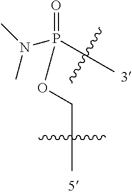 [SMILES: *COP(*)(=O)N(C)C] |
| A2 | PMO⁺ (unprotonated form depicted) | 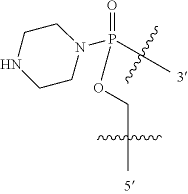 [SMILES: *COP(*)(=O)N1CCNCC1] |
| A3 | PMO⁺ (protonated form depicted) | 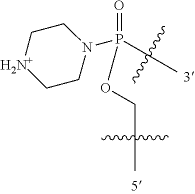 [SMILES: *COP(*)(=O)N1CC[NH2+]CC1] |

A “phosphoramidate” group comprises phosphorus having three attached oxygen atoms and one attached nitrogen atom, while a “phosphorodiamidate” group comprises phosphorus having two attached oxygen atoms and two attached nitrogen atoms. A representative phosphorodiamidate example is below:

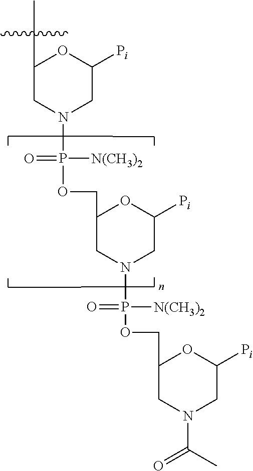

each Pᵢ is independently selected from H, a nucleobase, and a nucleobase functionalized with a chemical protecting-group, wherein the nucleobase independently at each occurrence comprises a C₃₋₆ heterocyclic ring selected from pyridine, pyrimidine, triazinane, purine, and deaza-purine; and n is an integer of 6-38.

In the uncharged or the modified intersubunit linkages of the oligomers described herein, one nitrogen is always pendant to the backbone chain. The second nitrogen, in a phosphorodiamidate linkage, is typically the ring nitrogen in a morpholino ring structure.

PMOs are water-soluble, uncharged or substantially uncharged antisense molecules that inhibit gene expression by preventing binding or progression of splicing or translational machinery components. PMOs have also been shown to inhibit or block viral replication (Stein, Skilling et al. 2001; McCaffrey, Meuse et al. 2003). They are highly resistant to enzymatic digestion (Hudziak, Barofsky et al. 1996). PMOs have demonstrated high antisense specificity and efficacy in vitro in cell-free and cell culture models (Stein, Foster et al. 1997; Summerton and Weller 1997), and in vivo in zebrafish, frog and sea urchin embryos (Heasman, Kofron et al. 2000; Nasevicius and Ekker 2000), as well as in adult animal models, such as rats, mice, rabbits, dogs, and pigs (see e.g. Arora and Iversen 2000; Qin, Taylor et al. 2000; Iversen 2001; Kipshidze, Keane et al. 2001; Devi 2002; Devi, Oldenkamp et al. 2002; Kipshidze, Kim et al. 2002; Ricker, Mata et al. 2002).

Antisense PMO oligomers have been shown to be taken up into cells and to be more consistently effective in vivo, with fewer nonspecific effects, than other widely used antisense oligonucleotides (see e.g. P. Iversen, “Phosphoramidite Morpholino Oligomers,” in Antisense Drug Technology, S. T. Crooke, ed., Marcel Dekker, Inc., New York, 2001). Conjugation of PMOs to arginine-rich peptides has been shown to increase their cellular uptake (see e.g., U.S. Pat. No. 7,468,418, incorporated herein by reference in its entirety).

“Charged,” “uncharged,” “cationic,” and “anionic” as used herein refer to the predominant state of a chemical moiety at near-neutral pH, e.g., about 6 to 8. For example, the term may refer to the predominant state of the chemical moiety at physiological pH, that is, about 7.4.

A “cationic PMO” or “PMO+” refers to a phosphorodiamidate morpholino oligomer comprising any number of (1-piperazino)phosphinylideneoxy, (1-(4-(c)-guanidino-alkanoyl))-piperazino)phosphinylideneoxy linkages (A2 and A3; see Table 1) that have been described previously (see e.g., PCT publication WO 2008/036127 which is incorporated herein by reference in its entirety).

The “backbone” of an oligonucleotide analog (e.g., an uncharged oligonucleotide analogue) refers to the structure supporting the base-pairing moieties; e.g., for a morpholino oligomer, as described herein, the “backbone” includes morpholino ring structures connected by intersubunit linkages (e.g., phosphorus-containing linkages). A “substantially uncharged backbone” refers to the backbone of an oligonucleotide analogue wherein less than 50% of the intersubunit linkages are charged at near-neutral pH. For example, a substantially uncharged backbone may comprise less than 50%, less than 40%, less than 30%, less than 20%, less than 10%, less than 5% or even 0% intersubunit linkages which are charged at near neutral pH. In some embodiments, the substantially uncharged backbone comprises at most one charged (at physiological pH) intersubunit linkage for every four uncharged (at physiological pH) linkages, at most one for every eight or at most one for every sixteen uncharged linkages. In some embodiments, the nucleic acid analogs described herein are fully uncharged.

The term “targeting base sequence” or simply “targeting sequence” is the sequence in the nucleic acid analog that is complementary (meaning, in addition, substantially complementary) to a target sequence, e.g., a target sequence in the RNA genome of human peripheral myelin protein 22. The entire sequence, or only a portion, of the analog compound may be complementary to the target sequence. For example, in an analog having 20 bases, only 12-14 may be targeting sequences. Typically, the targeting sequence is formed of contiguous bases in the analog, but may alternatively be formed of non-contiguous sequences that when placed together, e.g., from opposite ends of the analog, constitute sequence that spans the target sequence.

As used herein, a “target sequence” refers to a nucleotide sequence within the genome of human peripheral myelin protein 22 to which the antisense compound will bind under conditions suitable for such binding, e.g., physiological conditions. Examples of potential target sequences include sequences which comprise all or at least a portion of a 5′ terminal region, a transcription regulatory sequence (TRS), a translation initiation region, or an AUG region. A target sequence can typically encompass about 10 to about 30, about 20 to about 30, or about 20 to about 25 contiguous nucleotides of viral genome sequence.

As used herein, a “cell-penetrating peptide” (CPP) or “carrier peptide” is a relatively short peptide capable of promoting uptake of PMOs by cells, thereby delivering the PMOs to the interior (cytoplasm) of the cells. The CPP or carrier peptide typically is about 12 to about 40 amino acids long. The length of the carrier peptide is not particularly limited and varies in different embodiments. In some embodiments, the carrier peptide comprises from 4 to 40 amino acid subunits. In other embodiments, the carrier peptide comprises from 6 to 30, from 6 to 20, from 8 to 25 or from 10 to 20 amino acid subunits.

In certain embodiments, the carrier peptide, when conjugated to an antisense oligomer having a substantially uncharged backbone, is effective to enhance the activity of the antisense oligomer, relative to the antisense oligomer in unconjugated form, as evidenced by:
(i) a decrease in expression of an encoded protein, relative to that provided by the unconjugated oligomer, when binding of the antisense oligomer to its target sequence is effective to block a translation start codon for the encoded protein, or
(ii) an increase in expression of an encoded protein, relative to that provided by the unconjugated oligomer, when binding of the antisense oligomer to its target sequence is effective to block an aberrant splice site in a pre-mRNA which encodes said protein when correctly spliced. Assays suitable for measurement of these effects are described further below. In one embodiment, conjugation of the peptide provides this activity in a cell-free translation assay, as described herein. In some embodiments, activity is enhanced by a factor of at least two, a factor of at least five or a factor of at least ten.

Alternatively or in addition, the carrier peptide is effective to enhance the transport of the nucleic acid analog into a cell, relative to the analog in unconjugated form. In certain embodiments, transport is enhanced by a factor of at least two, a factor of at least two, a factor of at least five or a factor of at least ten.

As used herein, a “peptide-conjugated phosphorodiamidate-linked morpholino oligomer” or “PPMO” refers to a PMO covalently linked to a peptide, such as a cell-penetrating peptide (CPP) or carrier peptide. The cell-penetrating peptide promotes uptake of the PMO by cells, thereby delivering the PMO to the interior (cytoplasm) of the cells. Depending on its amino acid sequence, a CPP can be generally effective or it can be specifically or selectively effective for PMO delivery to a particular type or particular types of cells. PMOs and CPPs are typically linked at their ends, e.g., the C-terminal end of the CPP can be linked to the 5′ end of the PMO, or the 3′ end of the PMO can be linked to the N-terminal end of the CPP. PPMOs can include uncharged PMOs, charged (e.g., cationic) PMOs, and mixtures thereof.

The carrier peptide may be linked to the nucleic acid analog either directly or via an optional linker, e.g., one or more additional amino acids, e.g., cysteine (C), glycine (G), or proline (P), or additional amino acid analogs, e.g., 6-aminohexanoic acid (X), beta-alanine (B), or XB.

An “amino acid subunit” is generally an α-amino acid residue (—CO—CHR—NH—); but may also be a β- or other amino acid residue (e.g., —CO—CH₂CHR—NH—), where R is an amino acid side chain.

The term “naturally occurring amino acid” refers to an amino acid present in proteins found in nature; examples include Alanine (A), Cysteine (C), Aspartic acid (D), Glutamic acid (E), Phenyalanine (F), Glycine (G), Histidine (H), Isoleucine (I), Lysine (K), Leucine (L). Methionine (M), Asparagine (N), Proline (P), Glutamine (Q), Arginine (R), Serine (S), Threonine (T), Valine (V), Tryptophan (W), and Tyrosine (Y). The term “non-natural amino acids” refers to those amino acids not present in proteins found in nature; examples include beta-alanine (β-Ala) and 6-aminohexanoic acid (Ahx).

Representative oligomer-peptide conjugates are shown below:

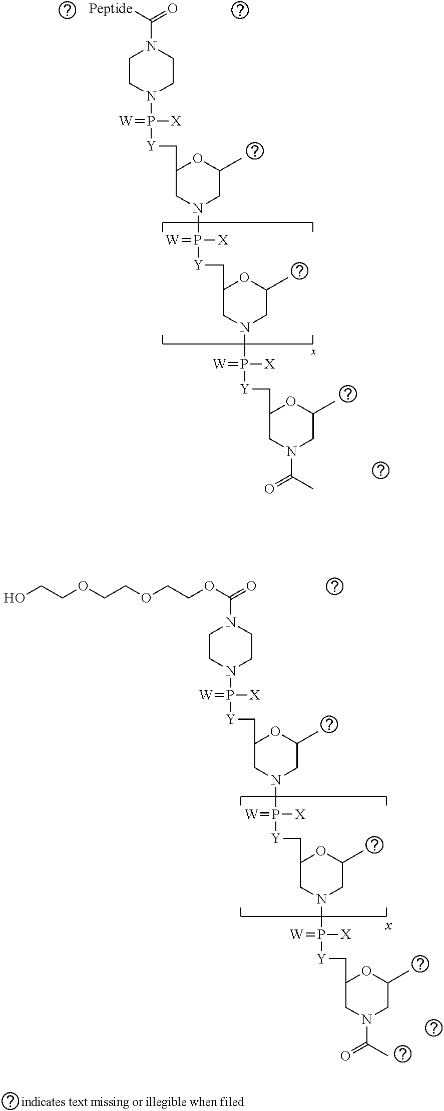

each morpholino oligomer is conjugated to a carrier peptide at the 5′ or 3′ end. W represents O; each X is independently selected from OH and —NR³R⁴, wherein each R³ and R⁴ is independently at each occurrence-C₁₋₆ alkyl; Y represents O; each Pᵢ is independently selected from H, a nucleobase, and a nucleobase functionalized with a chemical protecting-group, wherein the nucleobase independently at each occurrence comprises a C₃₋₆ heterocyclic ring selected from pyridine, pyrimidine, triazinane, purine, and deaza-purine; and x is an integer of 6-38.

An agent is “actively taken up by mammalian cells” when the agent can enter the cell by a mechanism other than passive diffusion across the cell membrane. The agent may be transported, for example, by “active transport,” referring to transport of agents across a mammalian cell membrane by e.g. an ATP-dependent transport mechanism, or by “facilitated transport,” referring to transport of antisense agents across the cell membrane by a transport mechanism that requires binding of the agent to a transport protein, which then facilitates passage of the bound agent across the membrane.

As used herein, an “effective amount” refers to any amount of a substance that is sufficient to achieve a desired biological result. A “therapeutically effective amount” refers to any amount of a substance that is sufficient to achieve a desired therapeutic result.

As used herein, a “subject” is a mammal, which can include a mouse, rat, hamster, guinea pig, rabbit, goat, sheep, cat, dog, pig, cow, horse, monkey, non-human primate, or human. In certain embodiments, a subject is a human.

“Treatment” of an individual (e.g., a mammal, such as a human) or a cell is any type of intervention used to alter the natural course of the individual or cell. Treatment includes, but is not limited to, administration of a pharmaceutical composition, and may be performed either prophylactically or subsequent to the initiation of a pathologic event or contact with an etiologic agent.

### II. Compounds

Provided herein is a chemically modified antisense oligomer having a targeting sequence that is complementary to a target region of the human peripheral myelin protein 22 (PMP22) pre-mRNA. In an embodiment, the antisense oligomer is a compound comprising a nucleic acid analog comprising a 5′ end, a 3′ end, and a targeting base sequence complementary to a target region of the human peripheral myelin protein 22 (PMP22) pre-mRNA.

In embodiment, the antisense oligomer induces skipping of one or more of exon 2 (SEQ ID NO: 2), exon 3 (SEQ ID NO: 3), exon 4 (SEQ ID NO: 4), or exon 5 (SEQ ID NO: 5) of the PMP22 pre-mRNA.

In another embodiment, the antisense oligomer has a targeting sequence complementary to a region within one of exon 2 (SEQ ID NO: 2), exon 3 (SEQ ID NO: 3), exon 4 (SEQ ID NO: 4), or exon 5 (SEQ ID NO: 5).

In another embodiment, the antisense oligomer has a targeting sequence complementary to a region spanning an exon/intron junction of exon 2 (SEQ ID NO: 2), exon 3 (SEQ ID NO: 3), exon 4 (SEQ ID NO: 4), or exon 5 (SEQ ID NO: 5).

In another embodiment, the antisense oligomer has a targeting sequence complementary to an intron region near an exon/intron junction of exon 2 (SEQ ID NO: 2), exon 3 (SEQ ID NO: 3), exon 4 (SEQ ID NO: 4), or exon 5 (SEQ ID NO: 5).

In a particular embodiment, the targeting region is PMP22 H2A (−25−1), PMP22 H2A (+1+25), PMP22 H2A (+25+49), PMP22 H2A (+30+54), PMP22 H2A (+35+59), PMP22 H2A (+38+57), PMP22 H2A (+40+59), PMP22 H2A (+40+64), PMP22 H2A (+42+61), PMP22 H2A (+44+63), PMP22 H2A (+45+69), PMP22 H2A (+46+65), PMP22 H2A (+48+67), PMP22 H2A (+50+69), PMP22 H2A (+50+74), PMP22 H2A (+52+71), PMP22 H2A (+54+73), PMP22 H2A (+55+79), PMP22 H2A (+56+75), PMP22 H2A (+60+84), PMP22 H2A (+65+89), PMP22 H2A (+70+94), PMP22 H2A (+75+99), PMP22 H2D (+15−10), PMP22 H3A (−15+10), PMP22 H3A (+1+25), PMP22 H3A (+15+39), PMP22 H3A (+24+48), PMP22 H3A (+48+72), PMP22 H3A (+65+89), PMP22 H3A (+74+98), PMP22 H3D (+17−8), PMP22 H3D (+22−3), PMP22 H4A (−10+15), PMP22 H4A (+30+54), PMP22 H4A (+60+84), PMP22 H4A (+90+114), PMP22 H4A (+100+124), PMP22 H4A (+110+134), PMP22 H4D (+22−3), PMP22 H5A (−8+17), PMP22 H5A (+18+42), PMP22 H5A (+37+61), PMP22 H5A (+55+79), or PMP22 H5A (+1271+1295).

In a particular embodiment, the antisense oligomer has a targeting sequence selected from SEQ ID NOs: 6 to 50. In an embodiment, the antisense oligomer comprises a targeting sequence complementary to a portion of, or induces skipping of, exon 2.

In a further embodiment, the target region of exon 2 is PMP22 H2A (−25−1), PMP22 H2A (+1+25), PMP22 H2A (+25+49), PMP22 H2A (+30+54), PMP22 H2A (+35+59), PMP22 H2A (+40+64), PMP22 H2A (+45+69), PMP22 H2A (+50+74), PMP22 H2A (+55+79), PMP22 H2A (+60+84), PMP22 H2A (+65+89), PMP22 H2A (+70+94), PMP22 H2A (+75+99), or PMP22 H2D (+15−10).

In a particular embodiment, the antisense oligomer comprises a targeting sequence selected from SEQ ID NOs: 6 to 29.

In an embodiment, the antisense oligomer comprises a targeting sequence complementary to a portion of, or induces skipping of, exon 3.

In a further embodiment, the target region of exon 3 is PMP22 H3A (−15+10), PMP22 H3A (+1+25), PMP22 H3A (+15+39), PMP22 H3A (+24+48), PMP22 H3A (+48+72), PMP22 H3A (+65+89), PMP22 H3A (+74+98), PMP22 H3D (+17−8), or PMP22 H3D (+22−3).

In a particular embodiment, the antisense oligomer comprises a targeting sequence selected from SEQ ID NOs: 30 to 38.

In an embodiment, the antisense oligomer comprises a targeting sequence complementary to a portion of, or induces skipping of, exon 4.

In a further embodiment, the target region of exon 4 is PMP22 H4A (−10+15), PMP22 H4A (+30+54), PMP22 H4A (+60+84), PMP22 H4A (+90+114), PMP22 H4A (+100+124), PMP22 H4A (+110+134), or PMP22 H4D (+22−3).

In a particular embodiment, the antisense oligomer comprises a targeting sequence selected from SEQ ID NOs: 39 to 45.

In an embodiment, the antisense oligomer comprises a targeting sequence complementary to a portion of, or induces skipping of, exon 5.

In a further embodiment, the target region of exon 5 is PMP22 H5A (−8+17), PMP22 H5A (+18+42), PMP22 H5A (+37+61), PMP22 H5A (+55+79), or PMP22 H5A (+1271+1295).

In a particular embodiment, the antisense oligomer comprises a targeting sequence selected from SEQ ID NOs: 46 to 50.

In an embodiment, the antisense oligomer is covalently linked to a cell-penetrating peptide.

In another embodiment, the cell-penetrating peptide is covalently linked to the antisense oligomer via a linker selected from a direct bond, a glycine, or a proline.

In yet another embodiment, the cell-penetrating peptide is selected from rTAT, Tat, R₉F₂, R₅F₂R₄, R₄, R₅, R₆, R₇, R₈, R₉, (RXR)₄, (RXR)₅, (RXRRBR)₂, (RAR)₄F₂, and (RGR)₄F₂, wherein A represents alanine, B represents beta alanine, F represents phenylalanine, G represents glycine, R represents arginine, and X represents 6-aminohexanoic acid.

In an embodiment, the antisense oligomer is selected from a peptide nucleic acid, a locked nucleic acid, phosphorodiamidate morpholino oligomer, a 2′-O-Me phosphorothioate oligomer, or a combination thereof.

In a particular embodiment, the antisense oligomer is a phosphorodiamidate morpholino oligomer.

In a further aspect, provided herein is an antisense oligomer having a targeting sequence that is complementary to a portion of one or more of exon 2 (SEQ ID NO: 2), exon 3 (SEQ ID NO: 3), exon 4 (SEQ ID NO: 4), or exon 5 (SEQ ID NO: 5) of the human peripheral myelin protein 22 pre-mRNA, wherein the antisense oligomer is an phosphorodiamidate morpholino oligonucleotide of Formula I:

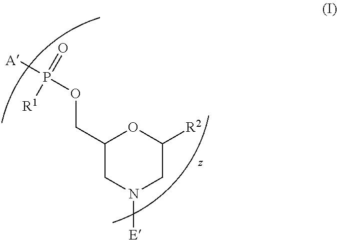

or a pharmaceutically acceptable salt thereof,
wherein:
A′ is selected from —NHCH₂C(O)NH₂, —N(C₁₋₆-alkyl)CH₂C(O)NH₂,

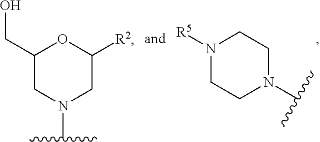

wherein
R⁵ is-C(O)(O-alkyl)-OH, wherein x is 3-10, and each alkyl group is independently at each occurrence C₂₋₆-alkyl, or R⁵ is selected from —C(O)C₁₋₆ alkyl, trityl, monomethoxytrityl, —(C₁₋₆-alkyl)R⁶, —(C₁₋₆ heteroalkyl)-R⁶, aryl-R⁶, heteroaryl-R⁶, —C(O)O—(C₁₋₆ alkyl)-R⁶, —C(O)O-aryl-R⁶, —C(O)O-heteroaryl-R⁶, and

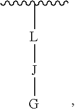

wherein Re is selected from OH, SH, and NH₂, or R⁶ is O, S, or NH, covalently linked to a solid support;
each R¹ is independently selected from OH and —NR³R⁴, wherein each R³ and R⁴ is independently at each occurrence H, —C₁₋₆ alkyl, or wherein R³ and R⁴ taken together represent an optionally substituted piperazine, piperidine, or pyrrolidine, wherein the piperazine has the formula of:

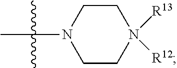

R¹² is H, C₁-C₆ alkyl, or an electron pair;
R¹³ is selected from the group consisting of H, C₁-C₆ alkyl, C(═NH)NH₂, Z-L²-NHC(═NH)NH₂, and [C(O)CHR′NH]ₘH;
Z is a carbonyl or direct bond;
L² is an optional linker selected from C₁-C₁₈ alkyl, C₁-C₁₈ alkoxy, and C₁-C₁₈ alkylamino;
R′ is a side chain of a naturally occurring amino acid or a one- or two-carbon homolog thereof;
m is 1-6;
each R² is independently selected from a naturally or non-naturally occurring nucleobase and the sequence formed by the combination of each R² from 5′ to 3′ is a targeting sequence;
z is 8-40;
E′ is selected from H, —C₁₋₆ alkyl, —C(O)C₁₋₆ alkyl, benzoyl, stearoyl, trityl, monomethoxytrityl, dimethoxytrityl, trimethoxytrityl,

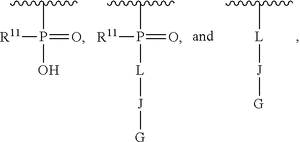

wherein
R¹¹ is selected from OH and —NR³R⁴,
wherein L is covalently linked by an amide bond to the carboxy-terminus of J, and L is selected from —NH(CH₂)₁₋₆C(O)—, —NH(CH₂)₁₋₆C(O)NH(CH₂)₁₋₆C(O)—, and

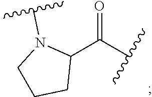 [SMILES: *C(=O)C1CCCN1*]

J is a carrier peptide;
G is selected from H, —C(O)C₁₋₆ alkyl, benzoyl, and stearoyl, and G is covalently linked to the amino-terminus of J.

In an embodiment of the antisense oligonucleotide of Formula I, the antisense oligomer induces skipping of one or more of exon 2 (SEQ ID NO: 2), exon 3 (SEQ ID NO: 3), exon 4 (SEQ ID NO: 4), or exon 5 (SEQ ID NO: 5) of the PMP22 pre-mRNA.

In another embodiment of the antisense oligonucleotide of Formula I, the antisense oligomer has a targeting sequence complementary to a region within one of exon 2 (SEQ ID NO: 2), exon 3 (SEQ ID NO: 3), exon 4 (SEQ ID NO: 4), or exon 5 (SEQ ID NO: 5).

In another embodiment of the antisense oligonucleotide of Formula I, the antisense oligomer has a targeting sequence complementary to a region spanning an exon/intron junction of exon 2 (SEQ ID NO: 2), exon 3 (SEQ ID NO: 3), exon 4 (SEQ ID NO: 4), or exon 5 (SEQ ID NO: 5).

In a particular embodiment of the antisense oligonucleotide of Formula I, the targeting region is PMP22 H2A (−25−1), PMP22 H2A (+1+25), PMP22 H2A (+25+49), PMP22 H2A (+30+54), PMP22 H2A (+35+59), PMP22 H2A (+38+57), PMP22 H2A (+40+59), PMP22 H2A (+40+64), PMP22 H2A (+42+61), PMP22 H2A (+44+63), PMP22 H2A (+45+69), PMP22 H2A (+46+65), PMP22 H2A (+48+67), PMP22 H2A (+50+69), PMP22 H2A (+50+74), PMP22 H2A (+52+71), PMP22 H2A (+54+73), PMP22 H2A (+55+79), PMP22 H2A (+56+75), PMP22 H2A (+60+84), PMP22 H2A (+65+89), PMP22 H2A (+70+94), PMP22 H2A (+75+99), PMP22 H2D (+15−10), PMP22 H3A (−15+10), PMP22 H3A (+1+25), PMP22 H3A (+15+39), PMP22 H3A (+24+48), PMP22 H3A (+48+72), PMP22 H3A (+65+89), PMP22 H3A (+74+98), PMP22 H3D (+17−8), PMP22 H3D (+22−3), PMP22 H4A (−10+15), PMP22 H4A (+30+54), PMP22 H4A (+60+84), PMP22 H4A (+90+114), PMP22 H4A (+100+124), PMP22 H4A (+110+134), PMP22 H4D (+22−3), PMP22 H5A (−8+17), PMP22 H5A (+18+42), PMP22 H5A (+37+61), PMP22 H5A (+55+79), or PMP22 H5A (+1271+1295).

In a particular embodiment of the antisense oligonucleotide of Formula I, the antisense oligomer has a targeting sequence (R²) selected from:

(SEQ ID NO: 6)
CTGCGAGGAGAGCGCTGGGCGTGAG, z is 25;
(SEQ ID NO: 7)
AAGTTCTGCTCAGCGGAGTTTCTGC, z is 25;
(SEQ ID NO: 8)
CAACAGGAGGAGCATTCTGGCGGCA, z is 25;
(SEQ ID NO: 9)
CTCAGCAACAGGAGGAGCATTCTGG, z is 25;
(SEQ ID NO: 10)
TGATACTCAGCAACAGGAGGAGCAT, z is 25;
(SEQ ID NO: 11)
ATACTCAGCAACAGGAGGAG, z is 20;
(SEQ ID NO: 12)
TGATACTCAGCAACAGGAGG, z is 20;
(SEQ ID NO: 13)
GACGATGATACTCAGCAACAGGAGG, z is 25;
(SEQ ID NO: 14)
GATGATACTCAGCAACAGGA, z is 20;
(SEQ ID NO: 15)
ACGATGATACTCAGCAACAG, z is 20;
(SEQ ID NO: 16)
TGGAGGACGATGATACTCAGCAACA, z is 25;
(SEQ ID NO: 17)
GGACGATGATACTCAGCAAC, z is 20;
(SEQ ID NO: 18)
GAGGACGATGATACTCAGCA, z is 20;
(SEQ ID NO: 19)
TGGAGGACGATGATACTCAG, z is 20;
(SEQ ID NO: 20)
CGACGTGGAGGACGATGATACTCAG, z is 25;
(SEQ ID NO: 21)
CGTGGAGGACGATGATACTC, z is 20;
(SEQ ID NO: 22)
GACGTGGAGGACGATGATAC, z is 20;
(SEQ ID NO: 23)
CACCGCGACGTGGAGGACGATGATA, z is 25;
(SEQ ID NO: 24)
GCGACGTGGAGGACGATGAT, z is 20;
(SEQ ID NO: 25)
ACCAGCACCGCGACGTGGAGGACGA, z is 25;
(SEQ ID NO: 26)
GCAGCACCAGCACCGCGACGTGGAG, z is 25;
(SEQ ID NO: 27)
GAACAGCAGCACCAGCACCGCGACG, z is 25;
(SEQ ID NO: 28)
GAGACGAACAGCAGCACCAGCACCG, z is 25;
(SEQ ID NO: 29)
AGGCACTCACGCTGACGATCGTGGA, z is 25;
(SEQ ID NO: 30)
CGATCCATTGCTAGAGAGAATCAGA, z is 25;
(SEQ ID NO: 31)
CGTGTCCATTGCCCACGATCCATTG, z is 25;
(SEQ ID NO: 32)
CCAGAGATCAGTTGCGTGTCCATTG, z is 25;
(SEQ ID NO: 33)
ACAGTTCTGCCAGAGATCAGTTGCG, z is 25;
(SEQ ID NO: 34)
GACATTTCCTGAGGAAGAGGTGCTA, z is 25;
(SEQ ID NO: 35)
GATGAGAAACAGTGGTGGACATTTC, z is 25;
(SEQ ID NO: 36)
TTTGGTGATGATGAGAAACAGTGGT, z is 25;
(SEQ ID NO: 37)
AGCCTCACCGTTTGGTGATGATGAG, z is 25;
(SEQ ID NO: 38)
CACCGTTTGGTGATGATGAGAAACA, z is 25;
(SEQ ID NO: 39)
CAGACTGCAGCCATTCTGGGGGAAA, z is 25;
(SEQ ID NO: 40)
GAATGCTGAAGATGATCGACAGGAT, z is 25;
(SEQ ID NO: 41)
AGAGTTGGCAGAAGAACAGGAACAG, z is 25;
(SEQ ID NO: 42)
TGTAAAACCTGCCCCCCTTGGTGAG, z is 25;
(SEQ ID NO: 43)
ATTCCAGTGATGTAAAACCTGCCCC, z is 25;
(SEQ ID NO: 44)
AATTTGGAAGATTCCAGTGATGTAA, z is 25;
(SEQ ID NO: 45)
TACCAGCAAGAATTTGGAAGATTCC, z is 25;
(SEQ ID NO: 46)
CACTCATCACGCACAGACCTGGGGAA, z is 26;
(SEQ ID NO: 47)
GCCTCACCGTGTAGATGGCCGCAGC, z is 25;
(SEQ ID NO: 48)
TTGAGATGCCACTCCGGGTGCCTCA, z is 25;
(SEQ ID NO: 49)
CCGTAGGAGTAATCCGAGTTGAGAT, z is 25;
(SEQ ID NO: 50)
CTCTGATGTTTATTTTAATGCATCT, z is 25.

In an embodiment of the antisense oligonucleotide of Formula I, the antisense oligomer comprises a targeting sequence complementary to a portion of, or induces skipping of, exon 2.

In a further embodiment of the antisense oligonucleotide of Formula I, the target region of exon 2 is PMP22 H2A (−25−1), PMP22 H2A (+1+25), PMP22 H2A (+25+49), PMP22 H2A (+30+54), PMP22 H2A (+35+59), PMP22 H2A (+40+64), PMP22 H2A (+45+69), PMP22 H2A (+50+74), PMP22 H2A (+55+79), PMP22 H2A (+60+84), PMP22 H2A (+65+89), PMP22 H2A (+70+94), PMP22 H2A (+75+99), or PMP22 H2D (+15−10).

In a particular embodiment of the antisense oligonucleotide of Formula I, the antisense oligomer comprises a targeting sequence selected from SEQ ID NOs: 6 to 29.

In an embodiment of the antisense oligonucleotide of Formula I, the antisense oligomer comprises a targeting sequence complementary to a portion of, or induces skipping of, exon 3.

In a further embodiment of the antisense oligonucleotide of Formula I, the target region of exon 3 is PMP22 H3A (−15+10), PMP22 H3A (+1+25), PMP22 H3A (+15+39), PMP22 H3A (+24+48), PMP22 H3A (+48+72), PMP22 H3A (+65+89), PMP22 H3A (+74+98), PMP22 H3D (+17−8), or PMP22 H3D (+22−3).

In a particular embodiment of the antisense oligonucleotide of Formula I, the antisense oligomer comprises a targeting sequence selected from SEQ ID NOs: 30 to 38.

In an embodiment of the antisense oligonucleotide of Formula I, the antisense oligomer comprises a targeting sequence complementary to a portion of, or induces skipping of, exon 4.

In a further embodiment of the antisense oligonucleotide of Formula I, the target region of exon 4 is PMP22 H4A (−10+15), PMP22 H4A (+30+54), PMP22 H4A (+60+84), PMP22 H4A (+90+114), PMP22 H4A (+100+124), PMP22 H4A (+110+134), or PMP22 H4D (+22−3).

In a particular embodiment of the antisense oligonucleotide of Formula I, the antisense oligomer comprises a targeting sequence selected from SEQ ID NOs: 39 to 45.

In an embodiment of the antisense oligonucleotide of Formula I, the antisense oligomer comprises a targeting sequence complementary to a portion of, or induces skipping of, exon 5.

In a further embodiment of the antisense oligonucleotide of Formula I, the target region of exon 5 is PMP22 H5A (−8+17), PMP22 H5A (+18+42), PMP22 H5A (+37+61), PMP22 H5A (+55+79), or PMP22 H5A (+1271+1295).

In a particular embodiment of the antisense oligonucleotide of Formula I, the antisense oligomer comprises a targeting sequence selected from SEQ ID NOs: 46 to 50.

In an embodiment, the phosphorodiamidate morpholino oligomer is covalently linked to a cell-penetrating peptide, wherein one of the following definitions occurs in the oligomer of Formula I:

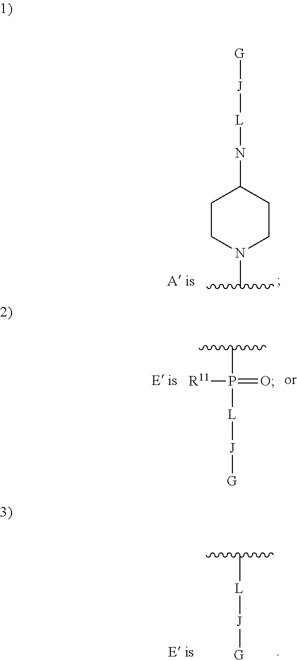

In another embodiment, E′ is selected from H, —C₁₋₆-alkyl, —C(O)C₁₋₆-alkyl, benzoyl, stearoyl, trityl, monomethoxytrityl, dimethoxytrityl, trimethoxytrityl, and

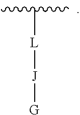

In yet another embodiment, A′ is selected from —N(C₁₋₆-alkyl)CH₂C(O)NH₂,

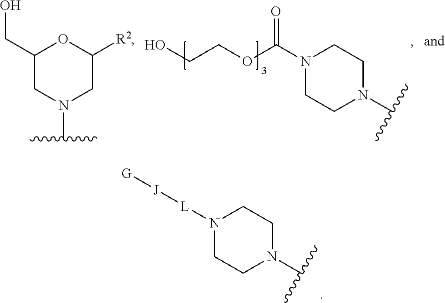

In a further embodiment, E′ is selected from H, —C(O)CH₃, benzoyl, stearoyl, trityl, 4-methoxytrityl, and

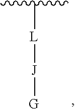

In another embodiment, A′ is selected from —N(C₁₋₆-alkyl)CH═C(O)NH₂.

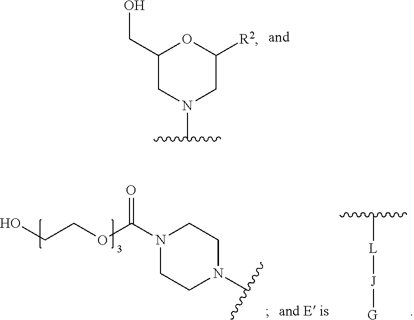

In an embodiment, A′ is

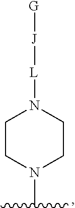

and
E′ is selected from H, —C(O)CH₃, trityl, 4-methoxytrityl, benzoyl, and stearoyl.

In a particular embodiment, the peptide-oligonucleotide conjugate of Formula I is a peptide-oligonucleotide conjugate selected from:

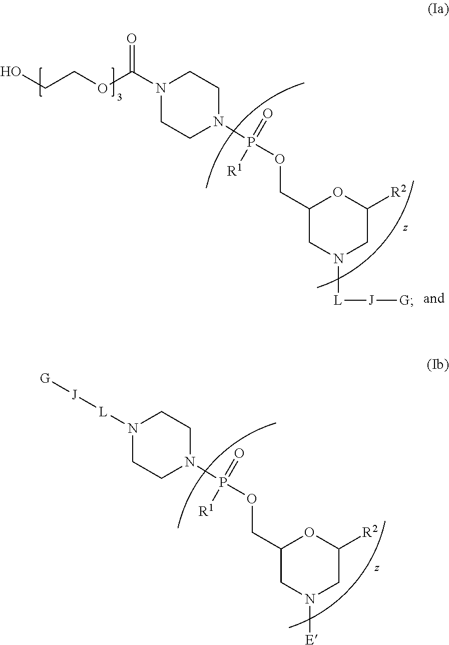

wherein E′ is selected from H, C₁₋₆-alkyl, —C(O)CH₃, benzoyl, and stearoyl.

In an embodiment, the peptide-oligonucleotide conjugate is of the formula (Ia). In another embodiment, the peptide-oligonucleotide conjugate is of the formula (Ib).

In yet another embodiment, E′ is selected from —C(O)(alkyl)ᵥ(O-alkyl)ᵤ-NHC(O)—R⁹, —C(O)—R⁹, and —R⁹, wherein u is 0-12, v is 0-12, each alkyl group is, independently at each occurrence, C₂₋₆-alkyl.

In an embodiment, A′ is

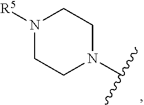

wherein R⁵ is selected from —C(O)(alkyl)_{w}(O-alkyl)_{y}-NHC(O)—R⁹, —C(O)—R⁹, and —R⁹, wherein y is 0-12, w is 0-12, each alkyl group is, independently at each occurrence, C₂₋₆-alkyl.

In another embodiment, E′ is —C(O)(alkyl)ᵥ(O-alkyl)ᵤ-NHC(O)—R⁹, wherein u is 0-12, v is 0-12, each alkyl group is, independently at each occurrence, C₂₋₈-alkyl.

In an embodiment, A′ is

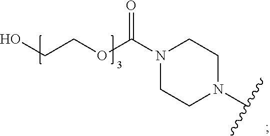

and
E′ is-C(O)(alkyl)ᵥ(O-alkyl)ᵤ-NHC(O)—R⁹, wherein u is 0-12, v is 0-12, each alkyl group is, independently at each occurrence, C₂₋₆-alkyl.

In another embodiment, A′ is-C(O)(alkyl)_{w}(O-alkyl)_{y}-NHC(O)—R⁹, wherein y is 0-12, w is 0-12, each alkyl group is, independently at each occurrence, C₂₋₈-alkyl; and E′ is selected from H, —C(O)CH₃, trityl, 4-methoxytrityl, benzoyl, and stearoyl.

In a further embodiment, the conjugate of Formula I is a conjugate selected from:

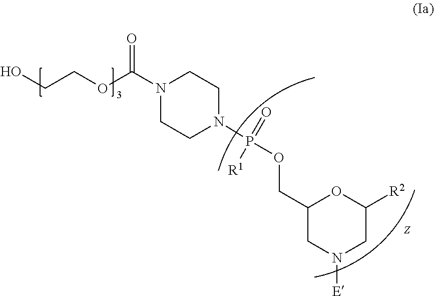

wherein E′ is —C(O)(alkyl)ᵥ(O-alkyl)ᵤ-NHC(O)—R⁹, wherein u is 0-12, v is 0-12, each alkyl group is, independently at each occurrence, C₂₋₆-alkyl;

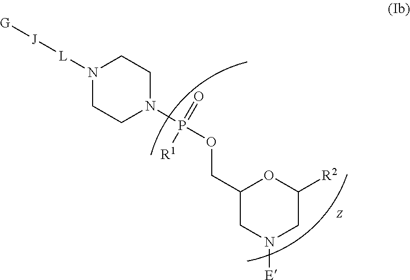

wherein E′ is —C(O)(alkyl)ᵥ(O-alkyl)ᵤ-NHC(O)—R⁹, wherein u is 0-12, v is 0-12, each alkyl group is, independently at each occurrence, C₂₋₆-alkyl;

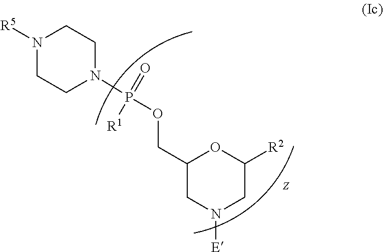

wherein R⁵ is selected from —C(O)(alkyl)_{w}(O-alkyl)_{y}-NHC(O)—R⁹, —C(O)—R⁹, and —R⁹, wherein y is 0-12, w is 0-12, each alkyl group is, independently at each occurrence, C₂₋₆-alkyl, and wherein E′ is selected from H, C₁₋₆-alkyl, —C(O)CH₃, benzoyl, and stearoyl; and

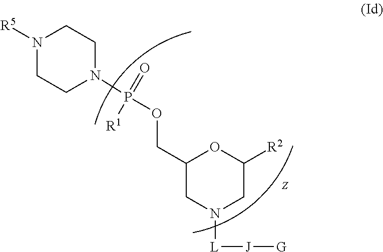

wherein R⁵ is selected from —C(O)(alkyl)_{w}(O-alkyl)_{y}-NHC(O)—R⁹, —C(O)—R⁹, and —R⁹, wherein y is 0-12, w is 0-12, each alkyl group is, independently at each occurrence, C₂₋₆-alkyl.

In a particular embodiment, the conjugate is of the formula (Ic):

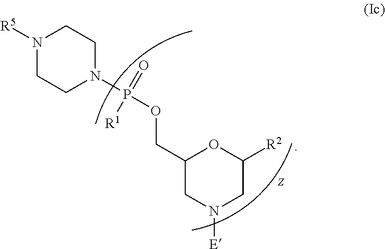

In another particular embodiment, the conjugate is of the formula (Id):

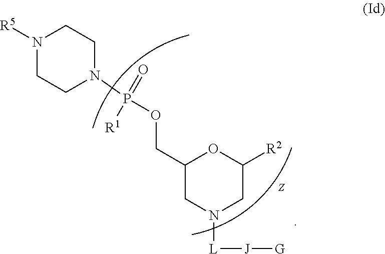

In an embodiment, provided herein is a compound having the following structure:

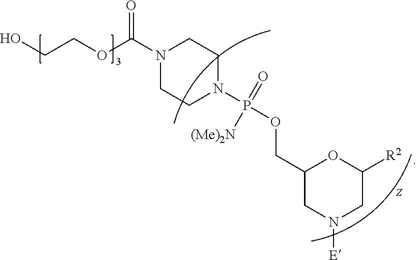

In an embodiment of the antisense oligonucleotide of Formula I, the cell-penetrating peptide is selected from rTAT, Tat, R₉F₂, R₅F₂R₄, R₄, R₅, R₆, R₇, R₈, R₉, (RAhxR)₄, (RAhxR)₅, (RAhxRRBR)₂, (RAR)₄F₂, and (RGR)₄F₂.

In an embodiment, each R₁ is N(CH₃)₂. In another embodiment, the targeting sequence is selected from SEQ ID NOs: 6 to 50.

In another embodiment, each R₂ is a nucleobase, independently at each occurrence, selected from adenine, guanine, cytosine, 5-methyl-cytosine, thymine, uracil, and hypoxanthine. In yet another embodiment, L is glycine.

In a further embodiment, G is selected from H, C(O)CH₃, benzoyl, and stearoyl.

In an embodiment, G is H or—C(O)CH₃.

In a further embodiment, G is H.

In yet a further embodiment, G is-C(O)CH₃.

In an aspect, provided herein is an antisense oligomer compound and a pharmaceutically acceptable carrier.

### Oligomer Chemistry Features

The antisense oligomers of the disclosure can employ a variety of antisense oligomer chemistries. Examples of oligomer chemistries include, without limitation, morpholino oligomers, phosphorothioate modified oligomers, 2′-O-methyl modified oligomers, peptide nucleic acid (PNA), locked nucleic acid (LNA), phosphorothioate oligomers, 2′-O-MOE modified oligomers, 2′-fluoro-modified oligomer, 2′-O,4′-C-ethylene-bridged nucleic acids (ENAs), tricyclo-DNAs, tricyclo-DNA phosphorothioate subunits, 2′-O-[2-(N-methylcarbamoyl)ethyl] modified oligomers, including combinations of any of the foregoing. Phosphorothioate and 2′-O-Me-modified chemistries can be combined to generate a 2′-O-Me-phosphorothioate backbone. See, e.g., PCT Publication Nos. WO/2013/112053 and WO/2009/008725, which are hereby incorporated by reference in their entireties.

In some embodiments, the nucleobases of the modified antisense oligomer are linked to morpholino ring structures, wherein the morpholino ring structures are joined by phosphorous-containing intersubunit linkages joining a morpholino nitrogen of one ring structure to a 5′ exocyclic carbon of an adjacent ring structure.

In some embodiments, the nucleobases of the antisense oligomer are linked to a peptide nucleic acid (PNA), wherein the phosphate-sugar polynucleotide backbone is replaced by a flexible pseudo-peptide polymer to which the nucleobases are linked. In some aspects, at least one of the nucleobases of the antisense oligomer is linked to a locked nucleic acid (LNA), wherein the locked nucleic acid structure is a nucleotide analog that is chemically modified where the ribose moiety has an extra bridge connecting the 2′ oxygen and the 4′ carbon.

In some embodiments, at least one of the nucleobases of the antisense oligomer is linked to a bridged nucleic acid (BNA), wherein the sugar conformation is restricted or locked by introduction of an additional bridged structure to the furanose skeleton. In some aspects, at least one of the nucleobases of the antisense oligomer is linked to a 2′-O,4′-C-ethylene-bridged nucleic acid (ENA).

In some embodiments, the modified antisense oligomer may contain unlocked nucleic acid (UNA) subunits. UNAs and UNA oligomers are an analogue of RNA in which the C2′—C3′ bond of the subunit has been cleaved.

In some embodiments, the modified antisense oligomer contains one or more phosphorothioates (or S-oligos), in which one of the nonbridging oxygens is replaced by a sulfur. In some aspects the modified antisense oligomer contains one or more 2′ O-Methyl, 2′ O-MOE, MCE, and 2′-F in which the 2′-OH of the ribose is substituted with a methyl, methoxy ethyl, 2-(N-methylcarbamoyl)ethyl, or fluoro group, respectively.

In some embodiments, the modified antisense oligomer is a tricyclo-DNA (tc-DNA) which is a constrained DNA analog in which each nucleotide is modified by the introduction of a cyclopropane ring to restrict conformational flexibility of the backbone and to optimize the backbone geometry of the torsion angle g.

In some embodiments, at least one of the nucleobases of the antisense oligomer is linked to a bridged nucleic acid (BNA), wherein the sugar conformation is restricted or locked by introduction of an additional bridged structure to the furanose skeleton. In some aspects, at least one of the nucleobases of the antisense oligomer is linked to a 2′-O,4′-C-ethylene-bridged nucleic acid (ENA). In such aspects, each nucleobase which is linked to a BNA or ENA comprises a 5-methyl group.

### 1. Peptide Nucleic Acids (PNAs)

Peptide nucleic acids (PNAs) are analogs of DNA in which the backbone is structurally homomorphous with a deoxyribose backbone, consisting of N-(2-aminoethyl) glycine units to which pyrimidine or purine bases are attached. PNAs containing natural pyrimidine and purine bases hybridize to complementary oligomers obeying Watson-Crick base-pairing rules, and mimic DNA in terms of base pair recognition. The backbone of PNAs is formed by peptide bonds rather than phosphodiester bonds, making them well-suited for antisense applications (see structure below). The backbone is uncharged, resulting in PNA/DNA or PNA/RNA duplexes that exhibit greater than normal thermal stability. PNAs are not recognized by nucleases or proteases.

A non-limiting example of a PNA is depicted below.

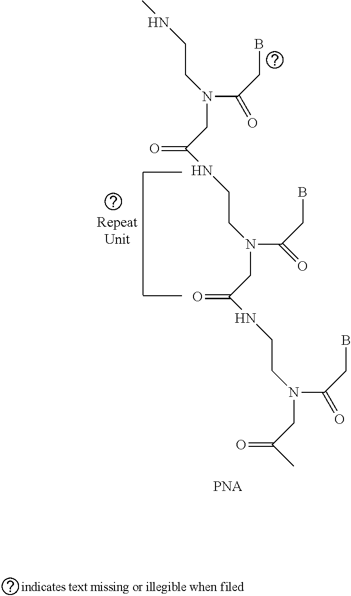

Despite a radical structural change to the natural structure, PNAs are capable of sequence-specific binding in a helix form to DNA or RNA. Characteristics of PNAs include a high binding affinity to complementary DNA or RNA, a destabilizing effect caused by single-base mismatch, resistance to nucleases and proteases, hybridization with DNA or RNA independent of salt concentration and triplex formation with homopurine DNA. PANAGENE™ has developed its proprietary Bts PNA monomers (Bts; benzothiazole-2-sulfonyl group) and proprietary oligomerization process. The PNA oligomerization using Bts PNA monomers is composed of repetitive cycles of deprotection, coupling and capping. PNAs can be produced synthetically using any technique known in the art. See, e.g., U.S. Pat. Nos. 6,969,766; 7,211,668; 7,022,851; 7,125,994; 7,145,006; and 7,179,896. See also U.S. Pat. Nos. 5,539,082; 5,714,331; and 5,719,262 for the preparation of PNAs. Further teaching of PNA compounds can be found in Nielsen et al., Science, 254: 1497-1500, 1991. Each of the foregoing is incorporated by reference in its entirety.

### 2. Locked Nucleic Acids (LNAs)

Antisense oligomers may also contain “locked nucleic acid” subunits (LNAs). “LNAs” are a member of a class of modifications called bridged nucleic acid (BNA). BNA is characterized by a covalent linkage that locks the conformation of the ribose ring in a C30-endo (northern) sugar pucker. For LNA, the bridge is composed of a methylene between the 2′-O and the 4′-C positions. LNA enhances backbone preorganization and base stacking to increase hybridization and thermal stability.

The structures of LNAs can be found, for example, in Wengel, et al., Chemical Communications (1998) 455; Koshkin et al., Tetrahedron (1998) 54:3607; Jesper Wengel, Accounts of Chem. Research (1999) 32:301; Obika, et al, Tetrahedron Letters (1997) 38:8735; Obika, et al, Tetrahedron Letters (1998) 39:5401; and Obika, et al, Bioorganic Medicinal Chemistry (2008) 16:9230, which are hereby incorporated by reference in their entirety. A non-limiting example of an LNA is depicted below.

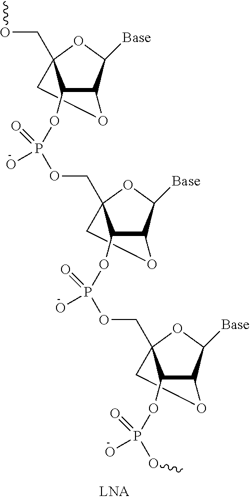

Antisense oligomers of the disclosure may incorporate one or more LNAs; in some cases, the antisense oligomers may be entirely composed of LNAs. Methods for the synthesis of individual LNA nucleoside subunits and their incorporation into oligomers are described, for example, in U.S. Pat. Nos. 7,572,582; 7,569,575; 7,084, 125; 7,060,809; 7,053,207; 7,034,133; 6,794,499; and 6,670,461; each of which is incorporated by reference in its entirety. Typical intersubunit linkers include phosphodiester and phosphorothioate moieties; alternatively, non-phosphorous containing linkers may be employed. Further embodiments include an LNA containing antisense oligomer where each LNA subunit is separated by a DNA subunit. Certain antisense oligomers are composed of alternating LNA and DNA subunits where the intersubunit linker is phosphorothioate.

### 3. Ethylene-Bridged Nucleic Acids (ENAs)

2′-O,4′-C-ethylene-bridged nucleic acids (ENAs) are another member of the class of BNAs. A non-limiting example is depicted below.

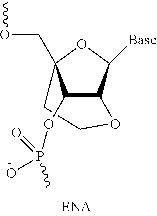

ENA oligomers and their preparation are described in Obika et al., Tetrahedron Lett (1997) 38 (50): 8735, which is hereby incorporated by reference in its entirety. Antisense oligomers of the disclosure may incorporate one or more ENA subunits.

### 4. Unlocked Nucleic Acids (UNAs)

Antisense oligomers may also contain unlocked nucleic acid (UNA) subunits. UNAs and UNA oligomers are analogues of RNA in which the C2′—C3′ bond of the subunit has been cleaved. Whereas LNA is conformationally restricted (relative to DNA and RNA), UNA is very flexible. UNAs are disclosed, for example, in WO 2016/070166. A non-limiting example of a UNA is depicted below.

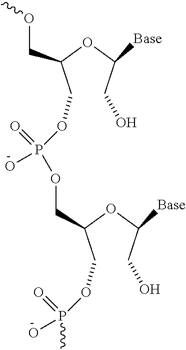

Typical intersubunit linkers include phosphodiester and phosphorothioate moieties; alternatively, non-phosphorous containing linkers may be employed.

### 5. Phosphorothioates

Phosphorothioates (or S-oligos) are a variant of normal DNA in which one of the nonbridging oxygens is replaced by a sulfur. A non-limiting example of a phosphorothioate is depicted below.

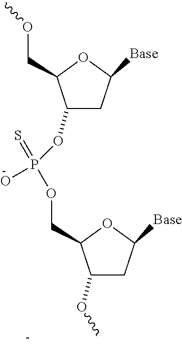

The sulfurization of the internucleotide bond reduces the action of endo- and exonucleases including 5′ to 3′ and 3′ to 5′ DNA POL 1 exonuclease, nucleases SI and PI, RNases, serum nucleases and snake venom phosphodiesterase. Phosphorothioates are made by two principal routes: by the action of a solution of elemental sulfur in carbon disulfide on a hydrogen phosphonate, or by the method of sulfurizing phosphite triesters with either tetraethylthiuram disulfide (TETD) or 3H-1, 2-benzodithiol-3-one 1, 1-dioxide (BDTD) (see, e.g., Iyer et al, J. Org. Chem. 55, 4693-4699, 1990, which is hereby incorporated by reference in its entirety). The latter methods avoid the problem of elemental sulfur's insolubility in most organic solvents and the toxicity of carbon disulfide. The TETD and BDTD methods also yield higher purity phosphorothioates.

In a particular embodiment of the antisense oligomer, the antisense oligomer is a phosphorthioate oligonucleotide conjugate of Formula II:

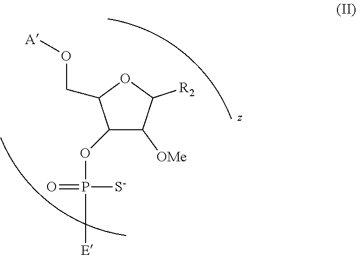

or a pharmaceutically acceptable salt thereof,
wherein:
A′ is selected from —NHCH₂C(O)NH₂, —N(C₁₋₆-alkyl)CH₂C(O)NH₂,

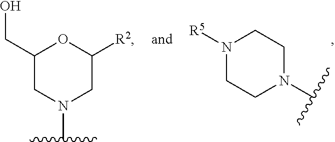

wherein
R⁵ is-C(O)(O-alkyl)ₓ-OH, wherein x is 3-10, and each alkyl group is independently at each occurrence C₂₋₆-alkyl, or R⁵ is selected from —C(O)C₁₋₆ alkyl, trityl, monomethoxytrityl, —(C₁₋₆-alkyl)R⁶, —(C₁₋₆ heteroalkyl)-R⁶, aryl-R⁶, heteroaryl-R⁶, —C(O)O—(C₁₋₆ alkyl)-R⁶, —C(O)O-aryl-R⁶, —C(O)O-heteroaryl-R⁶, and

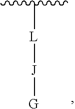

wherein R⁶ is selected from OH, SH, and NH₂, or R⁶ is O, S, or NH, covalently linked to a solid support;
each R² is independently selected from a naturally or non-naturally occurring nucleobase and the sequence formed by the combination of each R² from 5′ to 3′ is a targeting sequence;
z is 8-40;
E′ is selected from H, —C₁₋₆ alkyl, —C(O)C₁₋₆ alkyl, benzoyl, stearoyl, trityl, monomethoxytrityl, dimethoxytrityl, trimethoxytrityl,

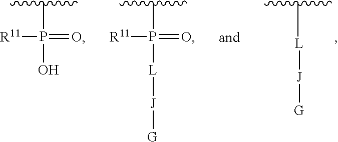

wherein
R¹¹ is selected from OH and —NR³R⁴,
wherein L is covalently linked by an amide bond to the carboxy-terminus of J, and L is selected from —NH(CH₂)₁₋₆C(O)—, —NH(CH₂)₁₋₆C(O)NH(CH₂)₁₋₆C(O)—, and

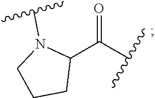 [SMILES: *C(=O)C1CCCN1* ; ";"]

J is a carrier peptide;
G is selected from H, —C(O)C₁₋₆ alkyl, benzoyl, and stearoyl, and G is covalently linked to the amino-terminus of J.

### 6. Tricyclo-DNAs and Tricyclo-Phosphorothioate Subunits

Tricyclo-DNAs (tc-DNA) are a class of constrained DNA analogs in which each nucleotide is modified by the introduction of a cyclopropane ring to restrict conformational flexibility of the backbone and to optimize the backbone geometry of the torsion angle g. Homobasic adenine- and thymine-containing tc-DNAs form extraordinarily stable A-T base pairs with complementary RNAs. Tricyclo-DNAs and their synthesis are described in International Patent Application Publication No. WO 2010/115993, which is hereby incorporated by reference in its entirety. Antisense oligomers of the disclosure may incorporate one or more tricycle-DNA subunits; in some cases, the antisense oligomers may be entirely composed of tricycle-DNA subunits.

Tricyclo-phosphorothioate subunits are tricyclo-DNA subunits with phosphorothioate intersubunit linkages. Tricyclo-phosphorothioate subunits and their synthesis are described in International Patent Application Publication No. WO 2013/053928, which is hereby incorporated by reference in its entirety. Antisense oligomers of the disclosure may incorporate one or more tricycle-DNA subunits; in some cases, the antisense oligomers may be entirely composed of tricycle-DNA subunits. A non-limiting example of a tricycle-DNA/tricycle-phosphorothioate subunit is depicted below.

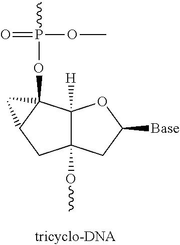

### 7. 2′—O-Methyl, 2′-O-MOE, and 2′-F Oligomers

2′-O-Me oligomer molecules carry a methyl group at the 2′-OH residue of the ribose molecule. 2′-O-Me-RNAs show the same (or similar) behavior as DNA, but are protected against nuclease degradation. 2′-O-Me-RNAs can also be combined with phosphorothioate oligomers (PTOs) for further stabilization. 2′-O-Me oligomers (phosphodiester or phosphorothioate) can be synthesized according to routine techniques in the art (see, e.g., Yoo et al, Nucleic Acids Res. 32:2008-16, 2004, which is hereby incorporated by reference in its entirety). A non-limiting example of a 2′-O-Me oligomer is depicted below.

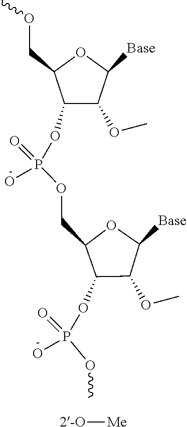

2′-O-Methoxyethyl Oligomers (2′-O-MOE) carry a methoxy ethyl group at the 2′-OH residue of the ribose molecule and are discussed in Martin et al., Helv. Chim. Acta, 78, 486-504, 1995, which is hereby incorporated by reference in its entirety. A non-limiting example of a 2′-O-MOE subunit is depicted below.

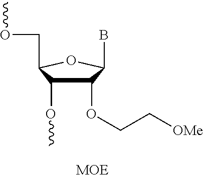

2′-Fluoro (2′-F) oligomers have a fluoro radical in at the 2′ position in place of the 2′-OH. A non-limiting example of a 2′-F oligomer is depicted below.

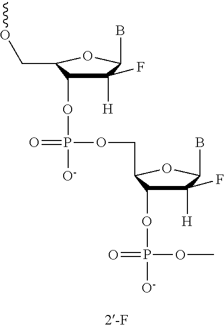

2′-fluoro oligomers are further described in WO 2004/043977, which is hereby incorporated by reference in its entirety.

2′-O-Methyl, 2′-O-MOE, and 2′-F oligomers may also comprise one or more phosphorothioate (PS) linkages as depicted below.

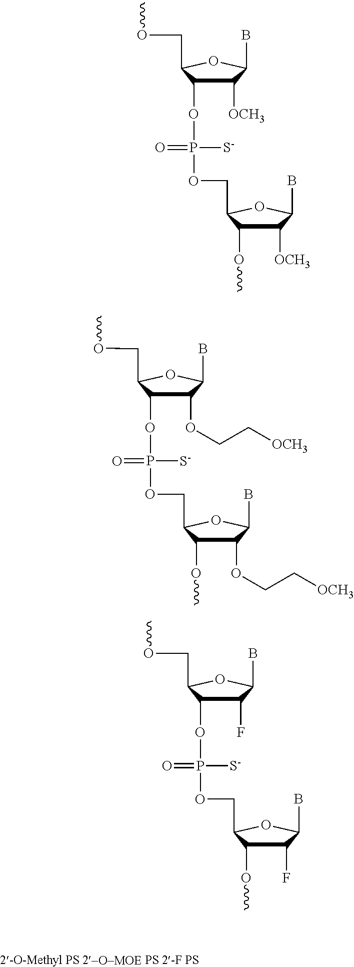

Additionally, 2′-O-Methyl, 2′-O-MOE, and 2′-F oligomers may comprise PS intersubunit linkages throughout the oligomer, for example, as in the 2′-O-methyl PS oligomer drisapersen depicted below.

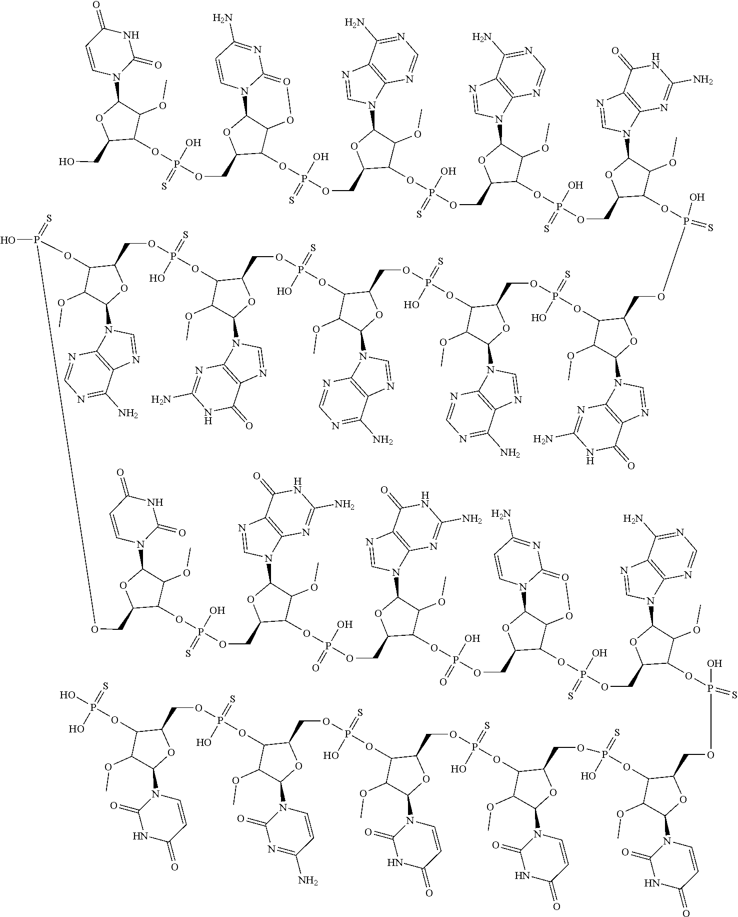

Alternatively, 2′-O-Methyl, 2′-O-MOE, and/or 2′-F oligomers may comprise PS linkages at the ends of the oligomer, as depicted below.

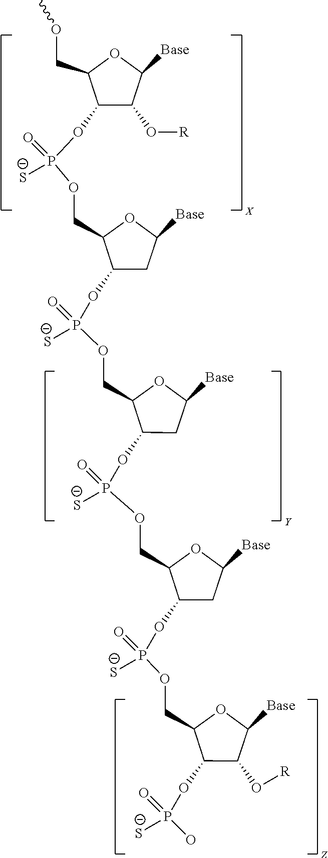

where:
R is CH₂CH₂OCH₃ (methoxyethyl or MOE); and
X, Y, and Z denote the number of nucleotides contained within each of the designated 5′-wing, central gap, and 3′-wing regions, respectively.

Antisense oligomers of the disclosure can incorporate one or more 2′-O-Methyl, 2′-O-MOE, and 2′-F subunits and can utilize any of the intersubunit linkages described here. In some instances, an antisense oligomer of the disclosure can be composed of entirely 2′-O-Methyl, 2′-O-MOE, or 2′-F subunits. One embodiment of an antisense oligomers of the disclosure is composed entirely of 2′-O-methyl subunits.

### 8. 2′-O-[2-(N-methylcarbamoyl)ethyl] Oligomers (MCEs)

MCEs are another example of 2′-O modified ribonucleosides useful in the antisense oligomers of the disclosure. Here, the 2′-OH is derivatized to a 2-(N-methylcarbamoyl)ethyl moiety to increase nuclease resistance. A non-limiting example of an MCE oligomer is depicted below.

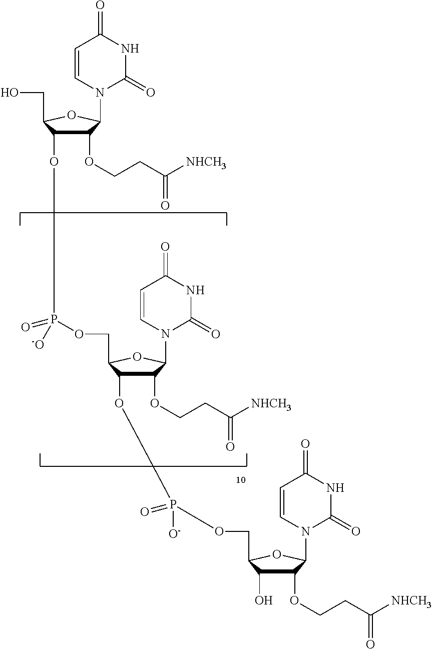

MCEs and their synthesis are described in Yamada et al, J. Org. Chem. (2011) 76(9): 3042-53, which is hereby incorporated by reference in its entirety. Antisense oligomers of the disclosure may incorporate one or more MCE subunits.

### III. Sequences for Splice Modulation of Peripheral Myelin Protein 22

In some embodiments for antisense applications, the oligomer can be 100% complementary to the nucleic acid target sequence, or it may include mismatches, e.g., to accommodate variants, as long as a heteroduplex formed between the oligomer and nucleic acid target sequence is sufficiently stable to withstand the action of cellular nucleases and other modes of degradation which may occur in vivo. Mismatches, if present, are less destabilizing toward the end regions of the hybrid duplex than in the middle. The number of mismatches allowed will depend on the length of the oligomer, the percentage of G:C base pairs in the duplex, and the position of the mismatch(es) in the duplex, according to well understood principles of duplex stability. Although such an antisense oligomer is not necessarily 100% complementary to the nucleic acid target sequence, it is effective to stably and specifically bind to the target sequence, such that a biological activity of the nucleic acid target, e.g., expression of encoded protein(s), is modulated.

The stability of the duplex formed between an oligomer and the target sequence is a function of the binding Tₘ and the susceptibility of the duplex to cellular enzymatic cleavage. The Tₘ of an antisense compound with respect to complementary-sequence RNA may be measured by conventional methods, such as those described by Hames et al., Nucleic Acid Hybridization, IRL Press, 1985, pp. 107-108 or as described in Miyada C G. and Wallace R B (1987) Oligonucleotide hybridization techniques, Methods Enzymol. Vol. 154 pp. 94-107.

In some embodiments, each antisense oligomer has a binding Tₘ, with respect to a complementary-sequence RNA, of greater than body temperature or in other embodiments greater than 50° C. In other embodiments Tₘ's are in the range 60-80° C. or greater. According to well known principles, the Tₘ of an oligomer compound, with respect to a complementary-based RNA hybrid, can be increased by increasing the ratio of C:G paired bases in the duplex, and/or by increasing the length (in base pairs) of the heteroduplex. At the same time, for purposes of optimizing cellular uptake, it may be advantageous to limit the size of the oligomer. For this reason, compounds that show high Tₘ (50° C. or greater) at a length of 20 bases or less are generally preferred over those requiring greater than 20 bases for high Tₘ values. For some applications, longer oligomers, for example longer than 20 bases, may have certain advantages.

The targeting sequence bases may be normal DNA bases or analogues thereof, e.g., uracil and inosine that are capable of Watson-Crick base pairing to target-sequence RNA bases.

An antisense oligomer can be designed to block or inhibit or modulate translation of mRNA or to inhibit or modulate pre-mRNA splice processing, or induce degradation of targeted mRNAs, and may be said to be “directed to” or “targeted against” a target sequence with which it hybridizes. In certain embodiments, the target sequence includes a region including a 3′ or 5′ splice site of a pre-processed mRNA, a branch point, or other sequence involved in the regulation of splicing. The target sequence may be within an exon or within an intron or spanning an intron/exon junction.

An antisense oligomer having a sufficient sequence complementarity to a target RNA sequence to modulate splicing of the target RNA means that the antisense agent has a sequence sufficient to trigger the masking of a binding site for a native protein that would otherwise modulate splicing and/or alters the three-dimensional structure of the targeted RNA. Likewise, an oligomer reagent having a sufficient sequence complementary to a target RNA sequence to modulate splicing of the target RNA means that the oligomer reagent has a sequence sufficient to trigger the masking of a binding site for a native protein that would otherwise modulate splicing and/or alters the three-dimensional structure of the targeted RNA.

In certain embodiments, the antisense oligomer has sufficient length and complementarity to a sequence in the PMP22 pre-mRNA, the sequence of which is provided in Table 2A below.

TABLE 2A
PMP22 pre-mRNA (SEQ ID NO: 1)

AATAAACTGGAAAGACGCCTGGTCTGGCTTCAGTTACAGGGAGCACCACCAGGGAACATCTCG
GGGAGCCTGGTTGGAAGCTGCAGGCTTAGTCTGTCGGCTGCGGGTCTCTGACTGCCCTGTGG
GGAGGGTCTTGCCTTAACATCCCTTGCATTTGGCTGCAAAGAAATCTGCTTGGAAGAAGGGGTT
ACGCTGTTTGGCCGGGTGAGTTTTATTGGCAAACTGTGCCTCTGGGTGATGTGTGCCTATGCTT
TACAAGAATTGCCTAATTTCCCACCCCCTGCAAGCCGCAAATGAAAAGGATTGCAGGAGAGATG
GTGCATTTGTGTTGGAATTGACTGGAGATTCAGAGGGCTTTTTATATCCTTGGTTAAAAGGTGGA
TATATACTCTGGCTGGGGAGGTGGGGGGCCATCTGAGAGCATCTAGAAACCCTAATGCATCCA
GATTGAAAGGAAGAAAGGAATCTAGATGCTTTTTCCCCCATGGAAAATAGCTGTGCACACACAG
CTGGCAGGTGGCCTTGGTAAGCAGGTTAGGGAGAAGCTGCCACCTGTGGCAGAGCCTTGGCC
AGCCGGGCTCTGGGTGTGGCAAGCCTGGTCAGCAGGCACTGAGTAGCACTTCCTCTGCCACCA
TTGAGAACCAGGCAGGAGGCCAGAGGCAGTGAGGGACAGATGGGTTGGGTTTACCTGTCTGG
CAGTATATGGTGTGGCTGTGACCTGGGTGGTTATTGATTAAACTATTGGGTTCAAAAGAAAGGAA
GAAGCGAGCTGTAGCACCCAATATATGCACTTTTCTGTATGTGTATTAGACTTTATTAAAAAGTTT
ATTTGAAAATCACAAAGGAAGGGAAAGAAAACCCTGAGTTAGATATGCTCATATTTCTAAAGTGC
TTACTTAGAACAGTCCTGAGTATGTTGTGATCACATAAGTGTTGGTTAAATAAATAAATGTCCAAA
TGGCATAAAACAAAGTAATAATTTCTAGAGCAAATCTAACTTATAAAATGACCTGTGGAGGAAAG
AAAACACTGCCATGTCTTTAGACTTTTTTTTCTACTTGCATATGCACTTTCATTTATAAATCTTTAT
CTATCTATCTATATCTATCTATCTATCTATCTATCTACCTACCTACCTACCTACCTATCAGAGTTAC
AAGCTACTTTAGTGCAGAGGTGGTCAGGTCTTCCCTGAAAGGTTGATGTGAGAGTTAAATGTGA
TATAATAATAGTGGTGGCAATTTGGGCACTGGGCCCTTATTATGTTGCAGACACGATGTTAACTG
CTTTGTACATATTGTCTCCTTCAATCACCACCATAAACCTTTAAAGCAGGCATTACTATTTCCTTTT
GCACCTGAGAAAACTGAAGCTCAGTGAACTTGAGAAATTGCCCAAATTCACAACAAAAGTAAGC
AGGGTGATTAGGTTTGTTGGAGCCCAGAGCCAGAGTGCCTAACCACTGCACCATACTGCCTCTC
ACAGACCATGCATATAAAGTCCAGGCAAAGTGCTTGGCATATAACGGGCACTGTAAATGCAGTG
TTTACAAGTACACTTCATTTTAATCTGATAACACGATAGTTTTAAAAAGATCATTGTTTGAAGATCT
TTTTCAAATTTATTTTTACTTACCGTAAGAATATACAATTAGCTAAAATAGCAGCTCCTTTCAAATC
GTAAAATAATATAGAATTATTTGAATCATTGAGAGTGATGAGCTTCTATGACACAGTACACTAGAG
GTAGGGAAGTGTGTGTGTGTGTGTGTGTGCGCGCGCGTGCGTGAACTGCTGCATTTTCAGGCA
GAAACCTTTAACATCCACATTCCTGCTCCCTGTCCCGTGCCTCAAGGCTGGCCTGCGCACGGGA
GTCTCAGTTGGGCGCGCCTCTGCCAAGCCGAGACTGAAGGGGGCTAGCCTCTCTCCCTGTAAC
GCTGGGCGGGCCACGTTAGGAGGCTATGAATCAGCTGATTTCCTTGGCTGCTCCAACCCCACC
TCAAATGGCCACCTCGCACCCGCCCGCCAAACCCCATGGCCAGGACTCCAGCCAAGGCTGACA
GCCAAGCCCGACTGCTGCAGGAACACTTTGCCTAGAGCTTATTCGGTGGTAGTCTGGTTTTGCC
TAGGCTAGGAGGAGCCCAATCCCAGACCATGCTATCCAGTAGTCGGCCGGACTTTTCTTCCCCT
AATTCGCACCCAAGAGGAGCCCGCTAGATCAATCCCCGCCAATCCTAGGAAGCTGGCATGCTT
CGTAGGTGCAGACAGTAATAGCGGGGACCGGCGCGGGGCAGGTGTGTCCGGCTAAGACGCCA
GGACAGGGCAGGGGCTCCAGGACCCGAGAGGAGAGGGACTTCTTCCAGCGCTCAAGCGCCCG
CTGCCCTCTATTAGTGGGAAAGGAATCCAGCCTCAGCCCCGCGCGGGCGCGGTCGGCGTCGG
CGGGCCCAGAAGCCCAGCCCTGGGCATCCGCTGAGCTACATTTGGCTGGGTCTTCCCAGAGTG
GGCTGAGGAGCCAGTTTCTCGGTCAACACTAGGTCTCCACGGGGCCAGGGGAGAAGGGAGGT
GGGAGGTGAGAAAGCTCAGCCGCCTCTGGTTTCGAGTAAAAGTCGCCGCGGTTTTGCAGGGAC
CGACTTTTTCTTGAGGCGCATTTAAGGCCAAGTGACTGTCTCCTGCCCTCCCTCTCTCCTGCCC
CCTCTCCTCCCTGAGTCCCGCCCTCCCGCACACGCTGACCCAGGGACACACCCTACTGCAGCG
ACGCAAACAGGGCGTTGTTCCCGTTAAAGGGGAACGCCAGGAGCCTCCCACTGCCCCCTTGCT
TCGCGCGCGCGCAGCCCCGCAGCGCAGCTTTGGCGGCGCCAGCAGCGGAGCCAACGCACCC
GAGTTTGTGTTTGAGGCCACCCTGAGGATCGGGACAGCTGTTCCTTTGGGCTGTAAGTGATTTG
GTGGGGAGAGTGAAGGAGATGGAGGAGAAATGTGGGCATCTTCCAGTGAGGGTGCCAGAAAG
CGCAGCGCAGGCGCGGGGCTTTGGCCAGCTCCCTGGGGCTTCTGTTTAGGGGCACAGGGTCC
CCTGTGTGTCCTGTTTCCCTCCAATGGGTCTTGGAGTAATACAACGAGGAGAGCCTTTATGGTC
TATAAGACCTTACAGGGCAAGGGTGATGGTGCTGGTGTCTGGATAGCGGATAAGGGCGGCCCA
GTTCTCGCCTTGCTGAAGAGGCGTTGACTCTGGGACACACTGGCAAAACAGTCCCTGTGGCGG
ACATCCCGAGAGTGTTGGACTCCTGTGGTTCCCCAGTTCCCAGCCTCCTGCCATCGGCTTAATT
CAAACCCTCTGGGAGTCATCAGAAATCCTTGTTTATTTCTTCCAGTGCATTCCAGAGTTTCTTTGA
ATGTGATGCTTGTGGAGAGGAACAGAGGGGCCTGGGATTGGGTACTTTCCAAACTGGGTTATTG
AAACGTTGTCTCCCTGGCCCAAGACTCTGCCTAAAATGGTCCCCAGAGTCTCAGCCAGATCTTT
CACAGCCATGCATGTCCTCTAACAGGTCATCCCAGCCTTCAGTTCCCCCAAAGTTTAAAATGAG
GGGCGAGGACCGCATTACTTGGGATAAATGTCCTGCCGGCTCTGACCGGCTGAAATTCTCGGA
CTCAGCCTTTTACCCTCTGTCTCTCTCTCCCGTCCGCTGGACATCTACTCCCTTCGGTCCAGGC
TCCTGGGCTCCTGTCCCACCGCACCCAGACCGGAGCTCAGGCTTGTTGGGGTTCGTGTCCTGG
CTTTGAGTGCCTGGGGTGCAGACTGGACCCCTAGGCGAGCACGTGGGTCTAGCGCAAGAGCA
GAATGCCCCCTGACCCCAGGGGCTGCCTGGGGAAGCGCGCGGTGGACGGGAAGCGCAGGGT
CCGGGCAACCCTCTCGAGCCATTCTTTTCTTTCCACACTACTCTGGCTCTGCACCACCTCTCCG
GAGCCGCCAGAGTCTGCGCGAGGCTGAGCTGGGGCCAGGACCGTTCCTCTACGCTGGCAGAG
TTGGGTGGAAACTTGGAGACAAACGGAATGCGGAGAGCACTGGGGTCTGGGAAACCAGCCTCC
TCGCCGCTGTCTCCCCCACCGCATGCCACGGCTGTCTCCAGGTCTCAGACGGAAATCTGGGCA
CCTACTCCCCTCACCCCTACCTCCACCCCATAGGGGAATTCACCATCTGAACCGGGGTCTCGGA
GAACGTGACCTCTCACCTTCCAGGGAGGTCGCCGGGAGGTGCTTGGGGTGGGTGGAAGCGTG
CAGTGGCCTCTGCTCATATTTTCTGGAAACCCCTCCGTTCCCTGGGTGGTTTTAGACGTGCGAA
CCGCTTGTTTTGTTTCCAAAAGCAAAAGATGTTCCGTTGCAGGCGGGCCCGGCTGGGCGCTGG
GCACTGGGCGCTGGTCCTGCAGGCGGCTGCTGCCCCCTCTCGGCGGCAGGCGGCGCGAAGG
CTCCTGACCCGCGCGGGCGGTCGGGCTGCGGGCGCTGGGCCAGGCCGGGCCTTCCGCTAGT
GCGCGGGACCCTCCCTCTGCGCGCGCCTCCGTCGCTCGGCCCAGTGCGTTCGGCCTCACGCC
CAGCGCTCTCCTCGCAGGCAGAAACTCCGCTGAGCAGAACTTGCCGCCAGAATGCTCCTCCTG
TTGCTGAGTATCATCGTCCTCCACGTCGCGGTGCTGGTGCTGCTGTTCGTCTCCACGATCGTCA
GCGTGAGTGCCTGGCGGGGAGGCTCCCTGCGCGGCCCGCCCTTCCCCATCTGGGTTCCCAGC
CCGTGCTCCTGCTGGTTCAGGACTGTGTTATTTGCAGACAGTTGGAAGTCTCAGACGTCCCAGG
GAAGTTTCTGGCAATCTGCCCCCTTCCAGTTGCTTTGAGAAAACGAGAGCAGATTCAGTGATAG
GAACCAAGCGAGCGCTGGGCTGGGTTAGCTGCGCAGGTCTCTATTTAAGCCAAGTAACTTCAG
AGCCGATCTAGGGTCCCCGACCTTCATTTGACAAGATTGTTTAACTTTTTTTTTTCTTGATGCAGC
CTCGTTGAAGAAGAGGAGTATTTATGATTTTTTTTTCTCCAAACCATAGTCAGATGGGTGAATTAA
TTTATAAAACACCCTTTTAGGACTTGAAATTGGAAAGTTAAAATGGGCTTTTCTGGAAAAGAGTTC
ACAGGTCCCATGTCGGCTTTATGGCATGAACACAACACATTGTTAGTAAAGCTTCCACTGGTAG
GAACAGGCCATCAGAGTAACTTCTGTTACAAAACTGTCCAGCCCATTAGATCTTAAAGTTATTTT
CTTGGCGATGAAGATGAGCAGACATTCAGGCAGTTTTCCAGTGGTGGCTTTCACTTCTTAATTTA
AGGCACATTGTAAGCATGATTTTCTTTTTCGTTTTCTTTTCTTTTCTTTTCTTTTTTTTTTTTTTTTTG
AGATGGAGTCTCCAGCCTGTCACCCGGGCTGGAGTGCAGTGCCATGATCTTGGCTTACTGCAA
CCTCTGCCTTCCGGGTTCAAGCCATTCTCCTGCCTCAGCCTGGGATTACAGGTGCCCGCCACC
ACGCCTGGCTAATTTTTTTTTTTTTTGTATTTTTAGTAGAGACGGGGTTTCACTATGTTGGCCAGG
CTTGTCTCAAACTCCTGACCTCATGATCCGCCCACCTCTGCCTCCCAAAGTGCTGGGATTACAG
GTGTGAGCCACCGTGCCCGGCCTGTAAGCATGATTTTCTAGTCTCTGTGAGAAATAATTATGTT
GGGGATTTGTGACTTAGTTTAACATTTACAAGCATCTTCACAGTATCTCCATTGTGCCTGGTGAT
GATGATATCAGACTGATAGGAGCTATAAACAAGGAAATAGACTGAGAGAGGCGGGCTTGGGGA
ATGATCCCGAGATGCTGGGAGGAGAGAAGGCTTGAATGCAGGTGTCTAAGGTTGAGTTCATGG
CTCCTTCTACTATAGTAAACATCTCTGCACATAATCGTTTCTGTGTGCATGTGGAACTTCTCCATT
TACAAGGTGCTTTTAAGTCATAAAACGTTGGCTCTTACCATGCAGGGGTGGGCGGTGTGGCTAG
GTGGATGCGGGTGCTTTTCGCCATCCCTGGGCCTTTCTCCTTCCCCTTTTCCTTCACTCCTCCCT
CCCTCCCTGACTCAGGATATCTATCTGATTCTCTCTAGCAATGGATCGTGGGCAATGGACACGC
AACTGATCTCTGGCAGAACTGTAGCACCTCTTCCTCAGGAAATGTCCACCACTGTTTCTCATCAT
CACCAAACGGTGAGGCTGGTTTTGTGCTCCATGAGCTTGTCCTCAGACGCCTGTAACACGTTTC
TCAGCCCAGAGCTGGAAACGCTTATTGGAAGACACAAGCCAGAATGTCGTTGCTGGGGTGGGG
TGGGATGTGACAGGGAGCCATGAGTCCAGGGAAAATGGGAGGGGTGGTGATCTCGCTGGGGA
AGCTGAGATCCTGGGCATCATTGTGATTCACTCTACCTAGAGACATGCCCTTAGTGCCCTCCTG
ATCACTTAGGATAATGCTTTTTGTGATTGAAAAAAATATCGACCTGGGAGCTGTGAAGGTGGTAT
GATAGCTAAGCCTTTGGCAGACCCCCTTGGGACATTGGATCAAACGTGTATCTTTGGTTTGGTG
GCTGTCTCTATTCAGAAATGTCCTGCCCCTTTCTGCTGCAGTGACAGTGGGGCTAGCTCACTGC
ACCGTCAGTTACATGGACACAAGACTGGCTGCCTTTCCAGGTCTTGGGCCTCAGTGATGCCACC
TAGCATCAAAACCTTTGAATGTTTCCTTGTTGCAAGATGGGGACTTGAAGCTATTTGCAAAACCC
TAAGCTAAGCCTTGGGGTCCTTAATCCAGAAGATCGTATTTCTCCTCTGTCCTCAGCATCTTTGC
TTGTGAGATATGGAAGCCAGGCTTCATGAGGGTCAAATTAAGGGATAATTGGCAGCAGAAGCAC
CAACTAGGTACCCAAGGCACCCAGAGGGAAAGGAACACCCAGCCAGGAGCCCTGTGTGTTTGG
ATGCAGACAGGTGGAGGTAAACAGGATGAGCTGTTTTGGTGGCTGGGTAGGCCATAGCGATTG
ATGTTAAGAGCCCGTGGACCTGGATCCACATGATCTTTTGTTCCAGAAGATGTGTCCTCAGGCT
GGAAGGACCTAGAGATACAGCAGAGCTTTCCTAACATCCTGGGTTTAAAAGGCCACGGAAATAC
AGCTTTAAATGTTGCTGGAGCTACTCATAGATCCAGTATATATGTTCAAGATGCTACACCAGACG
AGAGAAACCAGTTTAGATCCAGTTCAGTAAAACAGGCAGTGAAGTGGCCTTTGGTCCTGGCTTT
GCATAATCCAGTTGCTTTGCTGGACTTTCCCTCTTACAGCTCAGTACCCAGTAATACCCAAGCAT
GTCCCCAGCTGAAGCATTATAAACCAAATTCCTTGAACCACAGTACAAATAGAAACTGCTAAAAT
AATAATTTGGTAATTTACATGGAGAAGATACGCAAGGAGTAGAAAACCCTGCTTTATGTTTTTACC
TTCTGGGTTGGGGAGGGAGCAGAGAAAAGGTCCCAGTCCTATTGGAGTGAGAACAGTGCTTAA
TCTTGTCTCTGAGTGTGTGAAAGGCATTGAATTAAAGCCATCCCAGAAATTACGTGGTGGGTTTG
CATGGTGAGTTGGCAACTGAGTAAATGAAATACTCATGAGTGCCTGAGAATGTGACCGTAGCAC
TGCTTGTCCCCAAAGCCACAGCATCCGGTTTCTCCCCCTTTAGGGTCTGGCTGAAGTTCCCGGA
GCACTCCGGATGGGAGTCTCCGTACTCGCTACTGACACCTGGTGGTTACAAGTAGTGACTCAG
CCCGAAAAGGGCAGGCTTTGGTGCTCACAGGCGCCATCCCCAAGTGGCATCTGGCCACGCGG
CTTGGCACAGAATCCTGGACTCTTGGGCCCGGAAGGGGGCAGTTTTGGCAGGCGTGTTAAAAC
CCGGCGAAGTTTCAGCAGAAACGAGGCGGCAGAGGAGTTGCTAAGTTGTGCTTAAACCATCTG
CAGGAAAGAAAGCAATATTGACCTGTAGCATGTTACAGATTGACATTTGGTGTCTTCTGCCGTTG
GAAATAGCTGTCAGTTGGTGCTAGAAGCAGATCTCAAAGAGGCACTAGGGTTATTTTAATCAGG
AAACTTCTTATCTCTCAACTTGTTCCTTCTGTGCCAAGCTCATGTTCTTGAGTTCATTTACAATGC
CTCCTTAGTGTGGGTTTCAAATCTGCCTCGTTGCTTTCCGTGAGGACCCCAGAGTGTCTGTTTTC
CACATCTCCCTTCTCAGCTCTCACAATCAGGGCATTTTGAAAACATCTGGACACTGCACCTGAAC
ATGCTGGGTTTGTTTTCACGCCACTCAGGCTTAATCTAAATTTGAAATTTCCATTTACACTTCCCC
AGTAGTGGTTATCTCTGCCGCTATCTTCCCCAACGTGGCAGCACTTGCTACGACTTCAGCCTTA
AGTGGCGAATCCTCCAGGGCCTCTTTGATTGAGTTTAACAATTGTGGCTGCAGACAATAAGCTG
AAAAAAATGTGTACTTTTTAAAACATTATACTGTCTTTACAAATGAACTATGCTCATTGCGAACCAT
TTAACTTGTCTGTATTTGCTCTTAGAAAGTATATTTGAAAAAAAATACTTGTTAAGTAATCAAAAGT
AATTTGTTATTGACATCGATTTGTGAAGAATATGTTGCAAGTGTGAAAAAATAAATAGTGGTGCAA
TCTCGGCTGACTACAACCTCCACCTCCTGGGTTCAAGCGATTTTCCTGCCTCAGCCTCCCGAGT
AGCTGGGATTATAGGCGCCCAACACCACGCCTGGCTAATTTTTGTATTTTTAGTAGAGAGGGGT
TTCATCATGTTGGGCAGGCTGGTCTCGAACTCCTGACCTCAGGAGATCCACCCACCTCACCTCC
CAAAGTGCTGGGATTAGAGGTGTGAGCCACTGCGCCATGTGACTTGGTATTTTTTTCTGAAAGG
ACTCATTTTTCTGGTGCATGACCATGACACTTCACCTCTCTCTGGGGGTTGCCACTCATGTTGTT
TTCTACATGAATGGGGCCTCCTGGCATCGTGCCATGAGGCAGTCTAACATCTAGGTTGTGTAGT
TTGAAAGAGACACACCAGATCTTGCCCCCAGGCTACATCTTAATATTAGAAATAGTTTTAGATTTT
TAACTAACTGACTTTGACTCCGCCTGCCTTCTCGTTTTTCTAAATGTGTTTGTATACTTTCTGGCA
CCCCCTCCTTATCTGCCTCGTGTGGGCAGAGGTAAGAGAGGCTTAGTGTGGGGATATTCGTTAT
GATATTGTATCCAGTGCCTCTCCGGCCCAGGGAATACAAGGATCTGGAATGCATGGGCCTTGCC
TTGATGGTCCCGAGCCTGGTCGAAGAGTCAGGAGATAGATATGGAAGGGTGAATCACTCCCTT
GGAAGGCAGAGCAGGCCAGGTGCCCAAAGGGAGGCAGGCAGACAGCGTCTGAGTTCAGGGAG
GAAGCGGATGGGAAATCTCTGGTGGGAGGTAGAATCTGATCCATCCTCCAAAGGATGGGCAGG
ATTTAAGTCAATACAGGCAACGGGGAGGAGAATATTCCCTGTGACCTAAGGTAAGGGTAGAAC
AAGGCAGGGGATGGGGTAGACTGGTTGGCTAGTGTTCTGGTTAGGATTAGGATTGATTGTGTAT
AAGAGAACATCTTCAAATGATAGGGGCTTAAGTAAGATCATTTATTTTTCTCTCTTTTCAAAGAAG
TCTGGAAGTAGGTAGCCTGAGAGAGGTATGGAGGTTCCATGAAATTGTCAGAGATGCAGACACT
TTCTAGCTCATTTCTTTGCCATCTCTTGGGTTTGGCTGTCATCTTCAGGAAACAAAAGGCTGCTA
GAGCTCCAGCCATCTCATCCATGATTCAAGCAGTAGGGTGGAGAAAGGAGGTGGGGAAGAATG
AATCTCCTTTTCTTTTAATGAAATGTGACATGGTCACACCTACACCTATTTACAAGAGAGGCTGA
GAAATATAGTCATTCGGCCTTGGGGGAAGCATGGCTTTTCAAATCATAGAAACCTGGCAGCAAA
TACCTCTCTGTAAGCCTGGTGTGGAATACTTTCCTTGAGGGTTTGACCATCCCCTCCCATACCCA
ATCATACTCTTTTGCAATAGCTCAGTTGGGCCCTATGCAGTTCCATAAGTCCCAGTTATTTCAGC
ATTCTTCAAAGAGTCCATCTCTGCTGCATCCTGGGCTCTGTCAGCCACCCCACCATTCTCACATG
GGCTCTTCTCAGAAACTTTAGGACCTGTTGAGGCTTCTCTCTCTCTATTTCTACCACCTCTCCAG
GTATAAGTAAGTCTTATTATTATTATTGAGACAGAGTTTCACTCTTGTTGCCCAGGCTGGAGTTCA
GTGGCGCCATCTTGGCTCACTGCAACCTCCGCTTCCCGGGTTCAAGCAATTCTCCTGCCTCAGC
CTCCCGGGTAGCTGGGATTACAGGCATGCGCCACCACGCTTGGCTAATTTTTTGTATTTTTAGTA
GAGACGGGCTTTCTCCATGTTGGTCAGGCTGGTCTCGAACTCCCGACCTCAGGTGATCCACCT
GCCTCAGCCTCCCAAAGTGCTGAGATTACAAGCGTGAGCCACCATGCCTGGCCATCTTATTATT
ATTTAATGAGTCCAGACCTCTCTTCTGAATCCCAGACCTGTTTATCCAGCTGTCTCCTGGCCACT
TCCCTTGGGATATCTAAGAGAGGCCCCCAAGTCACTAGGCCAGAGACAGTCCTTATCTTCCTCT
GCCAATTCAACCCCTCTGTCACTTTCTAGTATTTTAGTGAATGCTGCTTCACCAGCCAGCAAGCC
AAACATCTAGAGTCTGCCTTGACTTGACGCGTTTCCTTCTTTCTCTTCTCAGCATTGTCACGGGT
CCCTCAGGCCACCCCATTGTTCCAATGCCTGTTGCCCACCTGTGGTCACGCTGCTTCCTCGTTT
GTCAGAGGCGCCAGGGCAGCCTCCTAACTAGTTTGCCTGAACCCTTTCTTGCTCCTCTGTGACT
CATTTTTCATATGGAATCCAGGCTGATATTTAAAAAAGCACAAACCTAATTATATCACTCCTCATT
TTGAAACCTTTCAGAGGCCGTTCATTGGTCTTAATCTTCAGATGAAAATCTTTAACAAGGCCCAT
CGGAATCTACCTGAATGATCCACTCCTGCTTCTGTTAAGTCTCACCTCCCATCTCTCTTCCCCTC
ATCTTGGAATTTCAGCGTTTTCTCACGTCCTGAAATGTGAACTTCCCACTTCGGGAGTCTCTCCA
TTTCTCTTCTGATCTTTCATGCAGGACTATTTCTTGAACAACTCTCGTGTACCAGAGACTATTGCA
GGTGCTAAGGTTACAAGAGTGAACAAGAGCTTATGTTTTACTGGACAAACCAGTCAGTAAACATG
GAGAATTTGGGGGCAATGGTAAGAACTATATAAGACAGGGTAATGGGATAAAGAGGGATTGCAG
TGGTGAAGGAAGGTCTCCTTGAGGAGTGGCCAGGAGCCAGCCATATGAACATCTCGGGGAGGT
TCCATACAGAAGGACCGTCAAGTGCAAGATCGCTGGGGAGGGAAGGAGCTTGGCGTAGTTGAG
GGACAGAGGGCAGGGCAGACTAGTGGGAACAGAGAGTGAGGGGTAAGTAGAATCACAGACCA
TCTCAAAGGTACCCCCCACCCCCACTAGGAAGGAGTTGGGTGCACATGGATGCATTTCTAATCT
TTGTGATTTCCTTAGACCTGCTGTCTTCCACGGGTTTCTCAGTGGTCCATTTGATTCCAAGTTGG
AGTGACAGTCCAGAAGCCTCTATAAGGCAGAATATATCAGAAATTGTACAAGGTAGAAATGCATC
GGTATATTTTCAGTGATTATTTTGGGTGACGGGATTCTTATCACTTTGGCTGTTTTCTTTCTTTTG
CTGCCTTACCTGTTTTCCTCATTGAGATCTTTTATTTTCACAATCAGAAAATAACTGTGAACATTTT
ATGTTAGAAAATTGGTTTTGAGTAGTAAGTAGGTTCCCTCTGCCTTGATTAAGAGAGTGGAGCCC
TGGGGCATGGCACGCAGGGTCCATGGCGTCGCTTTGAATGAGGCCAGTTCCTGGAGCTTTAGT
TTGCAAACTCATACAATCTGGAAGGCTCTGGTGAGGGGGAGGGGTGTGCCAAGCCTCCTCCCC
AAGACCCTATGGGTGAGTTTAAAATCTACTGACCCAAAGGATTCAGGCAAAACATTTGCCCTGCT
ATTGAATGAACCAGTGCTTTTTGTTTTGAAGTCTCCAAGATCCAAATATCAAGTCCAAGTTCTCAC
AAGCATTCCCCTGCTTTTTGTTTAATTTGTGTGGACCTGACGTGTAGCAGAATGGGTCAGAATAC
TGGAATTAAGCCACAGGCGGCTTGGGAGCCCAAGAGCTAGAAGGGCACAGTCCTGGCTTGCCC
TGGCCCCTGGCTGGTCTTCATCAGCTTCAGTGATGAGGCAGGACCCAGGCAGTTTGTCTTCAGT
AAAATCTGTCTCTCAGGGCCTGTCCCTGCTCACAAAGTCCTCTCTTGTCTCAGAAGAAAAGGTA
GTTGATAAACTTTTTCATGCTGAGGTGTTAGGCACAAAAAATGTGCCCCTTTTTGAATCACTTTTG
TTTTTAAAAAGGAGGCCAGTGCTCAACACCAAAGCATGAAGGGAATTGTTGAGCTTTCCTGTAAT
CTGTCCCCAAACGTCCTTGGCCACCTCCATTGTCTAAGGGCAGTACCTGCAGCCCCTCATTTCC
CAGTGACAACAGGTGGGTCTGCTCTGCCACAGTGTGTGGGGGGGACCAGCACCAGACGCTGA
GAAGATGACCACTTACTACTCTTCTTTTCCATTTAAACAGCAGCAGCAGCAGCAGCAGCAGCAG
CAGCAGCAGCAACAGCAAACTCATGACTATCAATAGGTTGTGTGAGGAATTAGATCAATGCTTTG
GGTTGAAAATTAGAGAAACTAAATCTTGCCAGTCTCAGCAGCCCCCAACCTGAGCCCTTGGTGG
TCTCCTATCATCGCTGTCTTCAAAACGATCCCAGACTTGTTTATCAATTTGGTGACACTGTCATTT
TGGCAAGGACACCAGACAATTTCCTTGTCTGTGGCTCAGCTTTTGGCCAGATTTTAGAAAACAC
GGGAGACCTTCTGCATTTATGGGTTCATGTTTTACATTTTAAAATCTTCGTGGCTCCCTCAGCCA
ACCCATAATCTCTACCCAAGGAATGGCTCTGGATTTTACAGGCTCTTATGAGATGCTGAGACCCT
GGTGGTACAAGGGAGGTACAGAGGACGGAATGTGTTGTCAACATGCCAGGATTTCAACCCTTA
GGGTTTCTCTGATGCCAATACCCAGGAATTAGTATGAAATGTTTGTTTGAACCTGTGTCTGAACT
GTATGACCTGGAATCTATAGTCTTTGCTTTAAAAAAACTTCAATTGTATCCCCAAGATTTCACCAG
CACCAAGAAAACATCCCTTGCATTTCAGGAGCAGGGACTGGCCATGGTGCAGCCTGAGGTTGT
GGATGTTGATCTTTGGTGCCCGAGAGAAGGCTGTCTGGGATGAAGCCTTCTGACTGTGGTCAG
GTCCCTGCCAGTGCTGGGGGCCACATGAGTGTGCAGTCATCCACACACAAGTGGCCCCCAACA
CTGGCAGGGACCCGGAGTCATCTGGCCCATCCCTCCTTCCTATCCCATCGAGCCACAGACCCC
TCTGCAATATGCCTACCACATGTTCATCCTACCACTGATGGGGAACTTATTACCTTCAAGAGAGC
CTCTTTTGTTTTTGAAGAGCTAATAGTGAGATCCATTCATGTGTTCTTCAACTCTATTGAGCCTGT
CCTATGTACTGGGCACTGTTCTAGAAATGGGGTGCACAGAGATGAACAAGAAAGAGCAGGTCC
CTGCCATTGTGCAGCTCACAAGGAGTTGGAGGAAACAGGCAATACACAATACATGCATCACTAT
GGGTAGTGGAACATGCTCTGCAGGAAACAAATAGGGTGATCCAATGGAAAGTCATCACTGGAG
GAATGAGCTGCTCAGGGAAGGCCTCTTCCTTCCATAGTGATGTGGTCTGAGATTTAGAAGACAG
GGAAGGACCAGTTATAGGAAGAACCATGGGAAGAAAATTCTAGGAGGTAAAGGGAAGAACCAA
GGCCCTGAGTTAGCTGAAGAAAGGAGGGTGGGGAATGCGGGAGAGAGGGATGGGAGCAGGAT
CGCTGGTGCCTCACAGGCTGTGGTACTGAGAAGGACAGTTGGGACATCATTGAAGGGTTTTAA
GCAGAAGAGTGTCATGATTTGATTCATGTTTTGTAAGCTCATTCTGGCTGTTGAGAATGAATTCT
GGGGAGAGGCAAGAGTGGAAACTGGGGACCAGTGAGGAGTTTGTGGTCATAGTCCAGACTGG
ACATGATGATGACACTGAGATGACAATGATGACAATGACAATGATGTTAAGAATACAAACAGCTC
ACGTAAGTGCCAGGCATTCTTTGAAAAGCAATGCATTTAATCCTCACAAGAACTCTAGGAGGTAG
CTGGTGGCTTAGCTAGGGAGGGGAAGTAGAGAAGAAAACAGGTCTCATGACAGGGTTTGCTGA
TGGGTTAGATGTAGGGAGTTAGGGAAGAATGATTCTAGATGACTCTACGTTTTGGGCTTGAGCA
ACTGGTGTGTAACTGAATCTTGGACTGAGATGGGGAGACTGGAGGAGGAACAGAAGATTTGCG
GGAAGGTGTAGAAATCAGGATTTATATTTTGACCATCAGAGAGGTCAGCAAATCACTTCAATTTA
GAAGTCTTTGGCTTAGGGGAGAGGTCTGGGCTGGAAATTCAAAGTCAAGAGTCATTCCTCTATA
CCGAAGGGTATTCAATGTTGTGGTGCTGAATACAGTTACTTGGGGAGGAGTTGAGAGCAGAAG
GCAGAAAAGGGGAACAGGGTTCAGAGCTAAGTCTTGGGCATGCCAGGGGTTAACGAGGGTACA
GGAAGGAGAGGAGACTGGGGATGAACAGCCAGTGATGAGGGAGGAATGCCAGGAGATATTGT
CACTTAAGAAATTACCTCGGCTGGGCGCGGTGGCTCACGCCTATAATCCCAGCACTTTGGGAG
GCTGAGGCGGGCGGATTACAAGGTCAGGAGATTGAGACCATCCTGGCTAACACGGTGAAACCC
TGTCTCTACTAAAAATACAAAACAAAATTAGCCGGGCGTGGTGGCCGGTGCCTGTAGTCCCAGC
TACTCGGGAGGCTGAGGCAGGAGAATGGCGTGAACCCGGGAGGCGGAGCTTGCAGTGAGCCG
AGATCGCACCACTGCACGCCAGCCTGAGCGACAGAGTGAGACTCCGTCTCAAAAAAAGAAATTA
CCTTGTTAAACCCAACACTGCCCCTCTTTTGTCCATTCACTTCTAGTTGCCTTCATTCATGCCTGA
TGTGCTGGACTTGGGTTTTATCAAGTTGCTTGGCACGTCCTTAAAGAATTTAAATCTCCACAGTG
CCTCCTGGACAATGACTGCAGGGTGACCCCCGTCTCTCCTCTACACGATGACTCTTCAAACAGT
TGACGATTGCAGTTTTTCTTTCCTTCCTTCTTTCAGTGGCTTCTCTGGTGCTGGTCCCAGCTGCA
TAAGCAGGGCCTCTTCTTGCCCTTCTGAAGTTTGGTCAGTGGTTTGGCTTGGCAGGCTGATGAG
CAGAGAATAAGGGAAGCCGCTCTCTCTTTGCCACACCATTCTCCTTCCACCCTCTCTAGTTTTTG
TGGATTCTAAGTCTGAAGAATGGTGGTAGAATAATTTAATTCATTAGATTGCAAGCATATCATTGA
CTTCTCTGCACCCCCAGTGTGTTGTAGTGGAAAAACTAGTAGTCTGGGCATCAGGGAATCCTTG
TTTTTCTGCACCGGGTCTGCCTGTAACAAATTAGGTAACCTGGGGCAGGTACCCTGGTTTCCTA
AAAGCAGAGCAGGCATTGAGGTAGGACCTGAAGTTGAGGTAGGACCTGCACATTGCATGGGTC
CCGGAGCCTCTGTTCATGTTCAGGATGTAATTGTGGCATCGAGGGATCCTAAAAGGAGGTGGC
CTCTGAGCTGGGCCTTGGCAATGGGGTAGAATTGCAGTAGGCAGAGGGAGCAGAGATGGGTTT
TGTGCTTGTAAACGGGCATTGGTTTACTGTCACTGGTGGGGGTAGCAGCTCTTTTGTTCTAGCT
CTTCATTTCCATAATGCGTGTTCTTTTTTCATACTTTAGGGGAAAGAAGGAGGAGAACTCTATTAT
TTCTATGGGGAGAATTCCTCCTAAACCTGAAGATCTTAAAACTAAGCGAATTATTCCCTGTTCATT
CTCCACTGATGCAGTTCATGACTGCAATTGCAACTGCCTTTCCCGTCATTTTCTATGGCCAAACT
CAGTGTTTTTAAGTGAGCTCTTCTTTTAAAAAACAAAAACAAAAACAAGTCTCCTGATAAATCCGT
TTGAAAGACACTAAGTTTTGAAAGTTAAGCAGGCTTTGATCATTCATGGTACATTGTGAATTACCA
AGGAGGGAGATTGATATCCTTCATCGTACAATGCACTCCCCTCCTTTTTCTTTTGTATCTGGTGG
AGTAAGTCTTCCAAGAGCAATTCTTAGAGAAACAAAGCCTACTCTGTTTCCCTGTTTCTAGAGTTT
TCCAACAAGGGTATAAAAAAGCCTCAGACAGCTTACTATTATCCAGTAACCTCAGGTACCTCAGT
GGCTGCGTGTTATTCCCTTTAGAAGTCCAAAAACTCACTAGCAGAGCAAGAATATGGATGTCAAA
GTGCAGAACTAGACCCTGACAAACTAGCTCTGTGCCTGCCACAAAGCCTCTTAATTAGGAATGC
AGTTCATTGAATAACTGTATCAAAGTTAGCTGGAATACCTTGACATGAAGATTCCTTCCACTTACT
GAGCAGTTTGTGCCCACTAGTGGCCAGACACAGGCTTTGCTCTAGCTAAAATACCCCCAGGATT
ACCTGTTGAGAGAGCCGCCCACGTCACCTTCATCGCCTTTGTGAGCTCCATGCTGGCACATAGT
TGTCTCCATCTGTTTTGCTTTCCGCATGAATCATAGGCAAAGTTAGCCTGACCAGCAAGCCCATT
TCAAAGCCACCAGCTGGGGGAGAAGTTGAAGCCCAGGGGGCAAGACCCACGCTGGGCTTTGG
GCATGTTTGAGCTGGTGGGCGAAGCATATGGGCAAAGGCCACATTGTTTAGGATGGAGGCCTT
TCAGAGACTCAGCTATTTCTGGAATGACATTCATACTGAGAAATAAGGAAAATGGCGATCTGTGT
GATGGTTGTGGGTTGGGAGGTTTGGGCGTGGGAGTCCTGGTCTTGGGGTCATGTGTTTTGAAA
ACAGTCTAGCACTATGCAAATGGGAGGTGTTAATAACTCTTTGCCTCTGTGATTTACCCTCTCAT
GGCTTTTTCTCTCTTGGCCTCTCCAAGGTCTCTTCTGACTCTTGCAACTGTCCTTCCTTTCACTT
TCAACAAAACCTGTTCTCTTGAGTCAGAACAGTTTTATGAATTGCCCAGAGTGGGACTTGATATG
GGGCAGGGTTGGTTCCATGCTGGTAATGAAAGATGCACATAAACTGTTACATTGAAAAGTGCTT
CATACTTTCTGAGCCCTTAACTCACTAGGTTGTAGGCCCTGGGCTAAGAATATAGCTGAGGGGG
CTGATGTATTTCCTTCATTGCTCAGCTGCTCCTACAGCAGGAATCTGACCTTGAAATGAGCTTTG
ATCTTTGTGCAACTCGAGCTTCCCACTATCTGCTGGGGGAGCTAGGTCTGGGTGCTGCCACGG
CCCATGTCAGAAAGCTGTCAGCAGCTCTTATACCGGACACTCTCAGAACAGGAGGCCTGCAAAG
AGCTTTAGCTTTAGTTTTGTTGTTGTTGTTGTTTTTGAGACAGTCTCGCTCTGTCGCCCAGGCTG
GAGTGCAGTGGCGCCATCTCGGCTCACTGCAAGCTCCACCTCCCGGGTTCACACCATTCTCCT
GCCTCAGCCTCCCAAGTAGTTGGGACTACAGGTGCCCGCCACCACGCCCAGCTAATTTTTTGTA
TTTTTAGTAGAGACGAGGTTTCACCGTGTTAGCCAGGATGGTCTCAATCTCCTGACCTCGTGATC
CACCCGCCTTGGCCTCCCAAAGTGCTGGGATTACAGGTGTGAGCCACGGCGCCCGGCCTTTTT
TTTTTTTTTTTTTTTGAGACGAGTCTTGCTCTGTTTCCAGGCTGGAGTGCAGTGGTGTGATCTCG
GCTCACTGCAGTCTCCGCCTTCCGGGTTCAAGTGATTCTCCTGCTCCAGCCTCCCAAATAGCTG
GGATTACAGGCACATGCCACCACACCCAGCTAATTTTTGTATTTTTAGTAGAGACGGGGTTTCAC
CATGTTGGCCAGTATGATCTCGATCTCCTGACCTTGTGATCCACCCGCTTTGGCCTOCCAAAGT
GCTGGGATTACAGGCATGAGCCACCACGCCCGGCCAGAGCTTTAGTTTTTAATTCCAGTGCTCA
GCACCAAGGTTGGATATTGGCTTAAAACTTCCACTGGAAACTCCACAAATGTTTAGCAGGCCTC
CACTTTGCACCAGGAACTGAGTGTGGCCTGGAGGACAGCAGGTACTCAACATAAGTTCTACTAT
TTATTGCATACTTGCAAATTGGGTGGGGCGTGGGGAAGAGTATCGCCACACTGGGGCTTGTGTT
AATCTATAGTTTAGCCACTTGGGATGTTGGTGTCACACTAGGTTTGATCTAAATAGAACAGGTTT
GTAATTGACAGCATTTTCACTTGCATTATATTTCTCTGAACTCGAATCATTATTTTCTGTACAGCT
GACTTGTTTTGCTGTAGCAGAGTTTGGCAGATAATAGCTTGCATGTCGAATCCAACCTGCTTTTG
TAAATAAGATTTTATTGGATAAATGGCTATTGAAGGAATGGGTCACACCCACTCATTTACATCTTG
TCCATGGCTATGGCTGATTTTGTGATACAAAGGCAGAATTGAGTAGTTGTTAAGAAGACCGTATG
ACCTGCAAAGCCTAAAGTATTTGCTCTCTGATTCTTTATAGATAGTTTGCTATCTGTGCAGATATT
TGCACTCTGACTCTGTAGCACCCTTATGCTGGAGAGGAGATATGAATTGATACCAAAGTTGTATC
AAAACACTTTTGGAGTTCTGGCCAATGTGCTAATAACCTGGCTTGTGTGAACATTGTGGCTCTCA
TTTTCTTTTTTCTTTTTTTTTTTTTTGAGACGAGTCTTGCTCTGTCGCCCAGGCTGGAGTGCAGTG
GCGCAGTGGCGCGATCTCGGCTCACTGCAAGCTCCGCCTCCAGGGTTCACGCCATTCTCCTGC
CTCAGCCTCCAGAGTAGCTGGGACTACAGGCGCCCGCCACCACGCCCGGCTAATTTTTTTTTTT
TTGTATTTTTAGTAGAGACGGGTTTTCACTGTGTTAGCCAGCATGGTCTCGATCTCCTGACCTCG
TGATCTGCCCTCCTGGGCCTCCCAAGTGCTGGGATTACAGGCGTGACCCACTGCGCCCGGCCT
GTGGCTCCCATTTTCATACTGCTTGGTGTGACCCAGATGGAAATGCTCCAGAACCTGGCCCCTC
AATAACAGCCTGTAAATGCTTGTTTACCCTGTTTTCTCACATCAGCCTTTTAGGTGGGGTTATTTG
CACTATCCTTTAACAGCGCTTCAATAACAACTCTTTTGTTTGATCATTATGACAACCCAATAAAAT
ATATTAGTGCCTCAAGTACGTAAAGCAGAATTCAAGGCCAAAAAAATGTATGTGGCAAGTTAGGT
GGGCGGTCAGAAGAACTTGGCAACAGAGCCCAGCCCTGCCTGGGCTGTCCTAACCAGAGCACA
TAAATGCTGGAGAGAGTCAGGCTTTGCACGTGCTCACACGGGACCCTTACCTTGGACAGGATC
AAAGCCCTGTCATTTCTGGAGTGTGTGATATCTGCTGTCTCTTACTGCGCTGGTTGTTACCTGTC
TATTCCAGTTCTGATGAATGGCTCTGATTGGGGAGCAGCATCATCTGAGCTTAGAAATGTCCTCA
GGGCCCTGTGGGGTTCCCTTTGAGCAACAACGAGAGCTCTTGAATCTTGCACCAGCCTTTTGGA
GCTGGAATGGAGTTTGCTTTCTCCTGGGCTGGGCTCTGTTTTTTTTCCTCTCGCTGTCCTTGTGT
AGCTTTTGATCAATGGCACTGGAGGAAAGGAAAATCCCTTGTTGTTTTCCTGGGCATGTGGAAG
CCCAGTCCTGCCCAGAATTCCACTAGCCCTTTGAGTTTTGGAATCCTGACTCTTAGTAAGTCAAT
AGAGTGCTGCATCTGTTTTTACCTTTGTAATCTTTTAAATTGGTTTTAATTATTTAAGTAATACAAG
TATTATAATACATGAAAATATCCTCAACTGAAAGGCTTCTTGACCAAACTCTTCTTCGTTGCAACA
GTAGTTCCTGGGGGAACCCACTGACATCAATTTGACACAACTTTGGCTCTTTCTCTACGAATATT
TGCATGCCTGTGGATGTCTATACGCAATTACATAGTTTTTTGGTGTTTGTTTTCATAATGATGACA
TGTTGTGTGTGCACCTTACTCTTTTCATTGTCTTGGAGACCTTTCAAAGACTCTTCATTTTCTAAC
TCATGATTTTTTTAACTGCTGCAAAATATTTCATTGTGAGGATATACCATAATTTATTTTCCCTCAT
AGAAATGCTACAATCAATGTCCTTATTTATTTTCCTCTGAGTATATGTTCACTGTTTTTCCAGGATA
TAGTAGATAAGCACTTGAACACATATAGTGTCATTACGCTAGAGGTGTTACAATTTTTAAACTACT
TTTAATGGCAAAAATTACAATTACTTTTGTATCAACCTAATATATTCTGCCACAAAATGGCTGGTC
ACAAAAGGATTATAATAATAATATTATTATTATTCCTGTGAATGGTGTCTGCAGGTAGCTATCACT
TATTTGTTCTGCTGATATTATCCATTTTTTTTGAATTTTGCCTATTGGCTGGATGAAAATTGGTGTC
ACCCTCCTCATGGGTTTCTCTTGCATTTCCTTGGTTGCTAGTGAGGTTGGGCACCTTTTACGATG
TTTATTGGCTGTTCATATTTCTCCTTTGCTAATCACATGGCTTTTCAATTTTATACTTAGCTGTTTT
CTCTTTGTTCTGTCTGTAATTTAATTTTGTTAATGATGCGAACTAGAGATTTAATTTTATGTTTTAC
CAAATGAATAGCCAACACTATGTATTGTCTGTTGTATTTTTCCCCACTGGTTTATATGATGTTGTG
TATTATCTACCAAATTTACATGTATGCTTCCAAAGTCTTATTCCATCCCATTCTTTTATTTTTCTTTC
ATGATTAACACCATATTTTTGAATTGCTAAAACTTTATAATATAAATATAAGCTTTGTATGTCTATA
AATAAAATACATTTTATTAATTATAATTATATGTTATATAATTATAATATAAGCACTTGAACATATAT
ACTTCTATAATTATATATTATATAATTATATATCATACTTCTATGATATGTTCTATAATTATATATTGT
ATATATCATAGTTCTATGATATGTTCTATAATTATATATTATAAAGTTACAATTAATTGTATATTTAA
TTTATACCATATACTGTAATAAGCAAAATATAGAAATATGTTTTAAATCTGGTAGGTGGTATCTTG
ACTCATTGGTTTTATATGTTTTTCTGATTTTCTTTTTGTTCATTTGTTCGTTTTGGTTTTTAGTTGCT
GTTGTGTTTACTTCAAATTTTTTCTTATCTGTCATTGCTCATTGTTTTTTAGATGAATTTTTAATTGA
TTTTTTTCCAGTTCCAGTTGCAATATGGATTAGGACAGCATTGAATTTGTAGATTATTTTGGATGA
AATTGGCATCTTTATAACTTTGAGTGTTTCTATCCAGGAACTTAGTATGTTTCTTTAGTACTTCTTT
TATGTACTTGAATAAAATTTGTCTAACTTGTTGATGTATCCCCAAAGAACCAGACATTTTTATTAAA
TGTATTTTAATATTTTTTTCTGTCTCATTAGTCTGTGCTTTTATCCATATTAAGTCAATTAATTTTTG
GACTTATTTCATTGTTTTTTTTCTGGCTTCTTGAGTTGAAAGTGTAGTTCACTCTTTTTAAGGATTT
TTCTAGCAAATGCTTAGAAGTGTATACATTGTCCTCTGAGAACTGCTTTGGTTACAACCCACAGG
TGTGCCATGTGGCTCTCTATATAATTCCTTGCTGAAGAAGTTTTTACTTTTCATCTTGTTTTTTTTA
ATCCGAGGAATTATCTGGAACACTGTTTTTAAATTTCCAAATTTATTTTGGCTAGTTTGTTTTTAAT
GTCTAATTTCTGTTTGCCTTTAGGTATATGTTGAGCCTACATCATTTCTGCTTCTATATTTATTGAG
ATATTCTTTGTTGCCCTTATAGTCAGTTTGAAAAGCTGTCCCATGTGTGTTTGAAAATAACTTTGC
TATATAGACACAAGTTTTCTATACATCTATTAGGACAGATTTGTTAATCCTCTTATGTAATCTATAT
TCTTTCTTCTTTTTGGTCTACCTGACATCTATTTTGAGGAAGGAAGCTAAAGTCTTCTACTGTTAT
TGTGATTTTAACAATTTCTTCTCATATTTTGATTACTTTTTGATTTTAACTTTTTCAGGCCATGTTAT
TATATGCATAATGATTTATTTTCTTCAGTCATAGATCATGTAAACCAGAAGTAAAGATCAAATAAA
CCAAATCTTTCCTCTATGAAACAACCAAACAACTCTCTGGCCACTTTTATCCTTTCAACATAAGTT
CTATTTTTTATTATTAATTTATCTTCTTTCTTTTTTGTTAACATTTGCCTGAAGCTTCTTTTCAGAAT
CTTTTTTGTTTTTGACTGTTGCTTGTTTTAGGTGTATCTATTATATTAGAATATAGCTACAATGTCT
CTCTTTTTTTTTTTTTTTTTTTTTTTTGAGACAGAGTCTCTCTCTGTGCCCAGGCTGGAGTGCAGT
GGTGTGATCTTGGCTCACTACAACCTCCACCTCCTGAGTTCAAGCGATTCTCTGCCTCAACCTC
CCAAGTAGCTGGGACTATAGGTGTCCACCAGCACACCTGGCCAATTTTTTTGTATTTTTCGTAGA
GATGGGGTTTTGGCATGTTGGCCAGGCTGGTCTTGAACTCCTGGCCTCAAGTGATCTACCTGCC
TCAGCCTCCCAAAATGTCTTAATCAAAGATAAAATTCCATATCTTATGAGTGCCTTGAACCCATTC
ACATATAATGTGGTTGATGATAGACTTACTATCTTAATGCATGTGTTTTATTTATTGTGCTTATGGT
TGAAAACCATCTTCTTCATCTCTGGATCATCAGAGTCAACCAGAGGGCTTGGCTCAAAGCAGTA
GGTATTCAACAAATGCTCATTGAATAAAAATGTCCTTGATCCTCTAATTCTGATTTGATTCGAGAA
ATAACTTATGGCAAAGAGCTCAATTGTAATGGGGTTGGGACTAAAATTCAAGTTAATGTTGAAAG
ATGTTTCAAGAAGTATTTAGGTTATATTATTAAATATTAGACACTTTCTGTTTATATAGATTTAACAT
ATAAAGATTTTAAGTTTTCTGTGTATGTGAGGTCTCGTAGTAGTTTCTGCTGAAAATGACAGGGG
CGCGGGGTTAAGGGTGAAGACAGTATCTCAGGATTGGAGAAGGAAATCTAAGATATTGTCTCCA
CCAGCCATACTCCTCATGAACATGCACATGGAAGGACATTTCCCAGCCAAAGTGAATCCCTTTAT
TTGTTTTAGTTTCAGATTTGAGTCTCAGATTTCTTTGTAATTCCAGCTGCATCTACCAAAGTGCTA
GATAATTTTTTTATTTTAAGCAAACAATATTATGAGAACATTATCTTTATTGCAAAGTGCTCCTCCA
GAAGAACCTCTATGCTGGATAAAGACAAACATTTGATTAAGGTTATGTAAGAGTGAAATCAGGAT
GGCTCACAACCCATTAGCCGTCATTTTCTTACTTTAATTGACAAAGAAATTGAAAAAATGAAAGAA
GCATATGTTGTGCTTGAATGTGAAGAGTGCTAGACTAGAATCAAAGGCCTCTTAGATTTGGCTGT
GTCATTTGGGCTCATGAAAACACCTCTGGAAATCTCTTTCAGTGCAAAGGAGCTGGACTAGTTG
ATCTCTCAGGTTTCTTCCAACTCTAAAATATCTCCAGTCTGTGCCTTGGAAACATCTTAGGTGAAA
ATCTAGGAACAGTTAACCTAATTTGCACCCTTAAAATTCTGCCATGAGCTGCTTACAACTCAAAA
CAAGTTTATCTTACTCAGTTACTAATTATAAACCATCCAGATTTCAGAGCTGTGAGTACTGGGTGA
AGCATTGAAGGTATGCTTTTGAAGCCATTACATATGGCAGTTACTGAGCTGAAAGGATTAAATGC
TGCAGCTTCCCCAGTTGCCCTTCCTCCATGAGAGCAGTGCCTGCCCCCAGCATTCTGTGGCACT
TGGAAGACAAGACAGAGGCCAAATGCAGATTTTTACCCTGGGCTTCCCTCTACAGTGTGGAAC
TCAGGTTGTTTCTTCTTTCCTCCCTGAAATGACATGAGTTTGCAGCGGATGGTGAACTGAAGAAA
CCATAGGAGGCTCTGTCTTCTTGCCTGAATTTCAGTTGGAAGCTTGGAGATTTGGGGTTCAACA
GAGATAAGGAAGTGTAAGCCTTCATCCCGTCTGGTGGTTGGCGATCACACACCGCTCTGTGCTG
AGGCTAATGGCCATGATCAGAGTTGACCAAAAAAAAAAAAAAAAAAAAATACGGGTTGTCCAAGC
AAATTGATTTCCATACCTATAGAGAGCATACCTTTCTTCATCAGTATTTTCTTCCATTCTTCCAAAA
AATTACTTTGGGCTCTAACAGCCATTCCCGTGATCTTTACCTCTCCTTGGGGAATGCAGATAATT
TGAATAGTGGTTTTAAGCTATTTTTCTTGGAATACAGAAGTTCTGATAAGCCCTTCAAAGACCCCT
GAGGGCAAGAAGAGGGAAGGTAGTAGGCAGGGCTCAGGCCCTCTTTAACACAGACACATGTAC
ATAAGTAACACATTTGCACCAACACTGGAAGGAAATTCAATATATTTTAAAATGACTTTAACTCCT
AGTGATGGAACTATAGATATTTTTTTACAGCCCTTCCCTGAATGTTCACATGTGTTTTTTATATATA
TGTGTGTGTCACGTATTTGTATATGTGTATATACACATATATATAGAAACACGTACATATTTGTAT
GCATATTTGTACATATAGGTATATATGTATGTAATGTGTACATATTGGCACATGTGTGTGTTTGTA
TATGTATATATCAGTACATATAGATGTATGTATATATGTATATATTTTTTCCTCCAAAATAAACAAT
AAAGAATGTCTTCTTAGATTGCAAAAGTGAATTGTCTGAGTTCTGCTAAGGAAAAAGGACTTCTG
CTTCTGCTGCCTGTGAGGACTGTGCTGTCAGCAGTCATGGCCCTTCAGGCCCTGCACCTCGAG
GCAGAGCCCTCCCCCGGCCATGGCCAGCTCTCCTAACCAAGTGTCTCTTTCCCCCAGAATGGC
TGCAGTCTGTCCAGGCCACCATGATCCTGTCGATCATCTTCAGCATTCTGTCTCTGTTCCTGTTC
TTCTGCCAACTCTTCACCCTCACCAAGGGGGGCAGGTTTTACATCACTGGAATCTTCCAAATTCT
TGCTGGTAAGTTGTGGATGGTAAAGTCCATGTGGAAGCGGGGTGCATCCAAGTCTGCGGAATG
ATTAGTTTAGTAGAAGGATGTGGCCTCAGAATGACTGATGTTCATGAGTCTCCCCACTGGATGCT
TTCCATAAAGTGAGGGTGGGTGCTTGTATGTGTGGGTGTGTACCTGTATGTGTCTTAGAACTTG
GGACTTAGAACTCTCCCCTTCTCCCTGGAATGAGATGCATATGAAAGAGAACTTAGAGGATCTG
GAAGGAAGGTCCCCACCCAAGCCAGGCGTATCAACAGGAATGAAACTGCAATCTGGACACATA
ATCAGAGGTGAATACTGAGGCTATCTGTAGAGCAAAGGTCAGGCTTGAGAGCTGTTTCTGTAGA
TTACATTATGCCTCCAGAAAATGGCCCTGATGTGCTAAGAACTAGCAAAGTAGTTATCAGGTATG
TGTCTTCCACCAATAGGTAGTGATGAAGCCACACTGACAAATTCTCACCTTCCTTGCTTCCAGTT
CCTAGATTCTACTGGGCTTTGATTGACTGTTGTCATCCTCTGGTGTCTTCATTTTGACACTCTTG
TGTCACATATTGTCATTTCCAAACATGGGGCTATGACAACACATGAAAACACATGAGAGGTCTCC
TTAATCTCCTGCCTAAACTGTCTTCAAGTTCCCTCTTTAAATACGTTATTAATATGCATAGTGTGC
AGAGTCCTAGAAACTTCTGTTACACAGGGTGACATCTTCCAACTTTGTCTCTGGATTCTGCCTAG
CATCTTACATGCTTACATCTTACATCTTACATCTTACATCTTGCATTCTGCCTAGCATCTTACATTA
GCTCTTACATGTCTGTCTGTTGACTTACTGTTGACTGAACCAGCAGGGCATTGGAGAGAAGTAA
GAGCTAGATGTAGTGGTGGATTCTGTGGTCCAAATTCATAGATCACAAACTTCATATGTACCAGA
GTATGTCTAGGTACTGGGAGATGTTCTCAATTCTGACCCTCTGAGAGGGCAAAGGATGTAGCAT
CTCTTCTCTGAGTTGGTTGTCAGAATGCCCATGGTACCATTTCACCACTCTGTCCCCAGGAGCA
GTCATTGGAAGGTTGACGTAAATAGGGTTGTATGGGAAGACACAGCCCAAGGTTAGATGTTGGT
GACCTTGTCTAGAAGACAGAGAGTTCCCCTTTCCTGAAAAAAGGAAGTAAATGATTAACCACTTC
TCATTAAACACTCAAATACAACATTTCAATACTCATGGTTTTGAGATTTCAAAACCAGACAGTGCT
TTGCTACTTACACATGTCTTATGACACCAAGCCAAGCTCCTGGATGGTTGCTGGCTCTGTTAAAT
GACTAATTATGCAAGGAGATATCATTTCTAGGTACGTTAAAGTGAAGAGTTACCCTTACTCAATTT
TCAGTTGGAATAAAAACAACTGTAACATATTCTGGGGTTTCTTTTTTTTTTTCTCACTCGTTTTAGT
TTGATATCAAATCAAATAATGATCATATCCATTGCATCAGTGGATATGCCCTCAAGATAATATGGA
TTTAGAACCAGAACTTTCATAATGTATTTCTATTGAAATGTTAGTTTCATAAGCGATGATTGGGTT
TTCATGCCCATGTGTGAGATGTGCCTCGCTCAAACCTTGTTATGATTTGGCACGTTACCCATCTG
ATGTGAAAAAAATTACATTTTATTTGTACAGGCTCGTTATTTTACTGATGAATAATTTGAGCCCAC
CAGAGGATAAATGAATGACCAAGGTCACCCAGCTCATGACAGGGACGGTTGAGTGTTACACTGA
ATATAGTGAGGTACTTCTTATATTTTAAAGACAGAATGCACCAAAAAATTTAAAGAACACAAAATC
CAAGGCAGAAGCTCTGCCTTTTATATTATCTTTTATTGGAACTGATTTACAATGGAAGGTAAATGC
AAATTTGCACCATGTATTATTCTGAAGTTCCAAACATCTGTGATGAATACAAGCCTGTACTATAAG
ACCCAGTCACATTGAAAATATGGAGCTGAGAAGAGGTAAGCTGCTGTTGAATGGGCTCCTTGGG
ATAGCCAGTACCTTCATCTTCATTCATCCTGCTGAGCTGTTTCGGCTTTAAGTTCTTTAACAATGT
CTTTTTAGCAACATCATTACATCATTTTAGGCCAAAACTCAAAGTCCAGAGATAAGAACCCTTAAG
TCACTCATGTAACTGCACTGTGTGTTAAAAGTATTTCAGTTCAGCCAAACACTCTTCTCCTAGGTA
TTGCGATTTAAGTATATTTACTAATCCACTCTTGCTTCACTATTTTCATTCTCCTCCAAAGTCAATA
CAAGATGTTTAGAACTGTGCTGGAAGTGCAGAATTCCGAATGTAAAAGCGCATGACTTTGTCCTC
TTTATCCCCTTTACATCTAGCTGCTTACGTCTCATGAAACTGAATTTTCAGTTATCTGTTGGTCCA
CATTTGAATAAGAAATTATCTTGAATTTGAAATGCTGAGCTGTAATAGCAGTTGTAATTTGTAAGT
CCTGAGAGTGTCCTGTCCTCCGTTGTTAATCCCAGTCAAGATCATCTGAGAGTTGGTCTCCAGG
GAACCTCAGATCTCTAGGATGTTGCACTGGAAATGGCTGCAGGATCTTTCCACTAATTCTGAGAA
CTGAAAGAGTTAGGAACTCTATTTGGAGAGTTCCTGGTTCCCTTATCGTGTGACAGTTCTAAGTC
AATTTTGTCATGTGGTTTTCTGACTCACAGACTAGTAAGAAGTTAGTAATTAGAGAGCTAAGTAG
ATTAGGGTTGTTGGAGATGAGAAACCCACGTTTTGGGAAAACCTGGCAAGTGACAACTTAACAT
CAAGGAAGTAGTCAGAAAAGCTAAAACTGACAAACAGAAGGTAGAAGAAAGTAGCTCCACACTC
ATGGGATGTGAAATCTACAAGCGTGCATGCCCGGCAAATGCCTCTCCAATGCACTGGAGCGTTT
TAAGTGGAAACCACTAGAATCTCTGTGTAGTCTCCGGAAGTGGCTGTGAGGGCTGCATTATCTC
TGCACAGCTTCCTCTTGGTGGGCCCAGCTGTGATCTTTATGGATGGCACACATCAGCTTTCAGG
AAAAGCACATGAAAGGTGCTAGGGCTCTTGGAGCTGACTGTAGGTTTGGGAGTTGTCTGTCTCC
TTGCTCTAGATTACAGCTCTGTGTGTTGTGTGGGGTCTCCATGGTTTGCCAAATTATCTCTTCCT
CACTTAGCCACAAGGCTGACAGTTAGGAACTATCTCTTCTTGATTGCATTAGGTTGGCTGCTTCC
TGAATGCATATCAAAAGGCTCCTTCCTTTAGTTCAGTGCTTTCAACCTGAGCTGTGCATCAGAAT
CACCTGAGGTCCTTTAAAAAAAAAAAAAAGACAGTGGGGGGGGCGCAGTGGCTCACGCCTGTA
ATCCCAGCACTTTGGGAGGCTGAGGTGGGCAGATCACTTGAGGTCAGGAGTTCAAGACCAGCC
CGGCCAACATGGTGAAACCCTGTCTCTACTAAAAATAAAAATAAAAAAATAGCCAGGCGTGGTG
GTGCGTGCCTGTAGTCCCAGCTACTTGGGAAACTGAGGCAGAGAAGAATCACTTGAACCTAGG
AGGTGGAGGTTGCAGTGAGCCAAGATCATGCCACTGCACTCCAGCCTGGGGGTGACAGAGTAA
GACTGTCTCAAAAAAAAAGAAAAAAGAAAAGAAAAAAGACAGTATTCAGGTCTCATCCCTGGAGA
CTCTCATTTAATAGTTGTGGGATGGATCCACTGCCTAGGTGACTCTGATGTGCATTTAGGGTTGG
GAACCACTGACATAGCCATTAAACTGTCCCTAATCCCACTGTAAGGTTTTCTAGGATATTTTCCC
AGAAATAACTAAACCACCTTCTTAGAGAAGGAACATCCTGATCCTGCGTCTGGACTTTGGAGTCA
TTCTTATTTTCAGAACCTATAGCCATCTATTCCTTGACAAGATCTGTTGGGTTGGGTTGCACTAGA
CTGGAAAACATCAAGAAATTAATCTAGACACAGACCTAAAAGGAAGATTTGCACGTTTTGATTTAT
TTTACTGCTTACACCCAGCATCAAGCTCCATGTGGGGCACATAGAGGAGCCTGAATAAAACTATT
ATTGGTTGGATGATTAGATAATTGTGTTTCCTCGGCAGAATCAAACTCAAGTCAAATGTGTGGTT
ACCGGGATTAAGAACAGAAAGAATAGAGGGGACTCCTGAGTGAGTTTCACTTTCTCTCTCTTTTT
GTTATTGTTGTTCATTTGTTTTGTTAATAGAAAGATCAATATAGCTATAGAGCATTTAAAATAAAAG
GATTTGGGCCAGGTGTGGTGGCGAATGCCTGTAATCCCAACACTTTGAAAGACTGAGGTGAGA
GGATTGCTTAAAGCCAGGAGTTCAAGACCAGCCTGGGCAACAAAGCGAGATGCCATCTCTACAA
AAAATGATTTAAAAATTAGTGGGGTGCCTTGGAGCACGCCTGTGGTCCCAGCTATTCAGGAGGC
TGAGGCAGGAGGATTGCTTGAGGCTGAGAGGTCAAGACTACAGTGAACTATGATCAGGCCCCT
GCACTTGCTCCAGCCTGGTGACAGAGTGAGACCCTGCCTCTCTAGGAAAAAAAAAAAAAGAATT
TGGTGGGGGGGGATTAATAAAAGTTCAGTGCCATCCTGTTCAGAGTGATTCAAAGGTGGCTTAA
GTCAAATACCACATTTAGAAATTTTTGTTAGGGACGGATGATTATGGATGTCTCACTGGCCATGC
CCAAATCAGAGTCAATGCTATGGGTGGCTTTCTAGGCAGCAATGGTCATTTGGATACTGAAAAT
GTAAAAGGAGTGGGCTTCAATCACAGAAACACAGAGAAGTCTCTTGCTTTCAGAGGAACAGGCC
TCACAGCCCCTTCCTGCCCTCCCTTGTTGCCCTGAGGATGAAGTGGGAGGGAGAAAAAGGCAC
CCACTCATCAACCCAACATCATAGCTGCACCTTCCAGTGACCACCCTGGTGGTCTCACTGACAT
CCCTTTCCAGCCAACTTCTCTCTGACTTCTGCCCCAGGCTCTCAAATGAAAGTGCTTTTGCGAG
GTTACCAAAGTACACTTTGAAGTCTTTAATTTGTTGGATCACTCTGCTCCTTTCAATACTGTTAGC
TACTTACTCTTTGAAGCTCTATTCCCCTGGCTTCTGAGACATCGCTGTGTTCCCACTTGCCTGAG
TGAACACACCAGGCGGTCATGCTGGCCTCTCCTCTCTCCCTCACCCCACAGCCAGCCTTCTACT
GAATCCTGCATTTGGGTAGCATCCTCCGTAGGCATTACGCCCATTTCTTTGTCTTGACTCACTAA
CCCCAGTGCAGCTATGCAAAGGCCTCCTCATGCACCTTCCAGCTACCTGCCTTGTCCCTGCCCC
TGAATCCATTTTCTGCACCATGGCCAGAGTGATCATTCTAAAACGTGCGTCTGATCATGTTTCTC
CTCTGCTGAAGTTTCCTCAGAGGCTTCTCATCACAGCTTCCTTAACATGTCATGTAAGGCCCTCT
CTTTCCTGACCCTTAGTGAGCTAATCAGCCTATTATATGAGAGTCCGTGTGGCTTCTCCAGTTCT
CCAGTTTGGACTCAGCGCCCGTCTATGCTCTTGGGGCATCTGGTGCATTTCCTTCCCTCTCTCG
AAATTCCATCGAACTATATTGAAATTATATGCAAATGTCTGTCTTCTAGACACACAGTCCTTAAGG
TAGGAGCCATGTCTCACTTATCTTGGTACATCTAGAGCAGCAGCTAGAACAGGGCCCGCATGC
AATAGGCACTCATTAAATGTTCACTGAACCAAAGGGAATGGAGATGATAAGGATGTACTGGGAA
TTCCCTCAGCTACTTCCTTTGACCTTGGCCCCTTGGGTGCTTCCTTCCAGAGGCTCTGGGCTCT
AATCACTCTCATGGGGTTGCAGTCACTGTTAAAGGAGCTTAAGACTTTCTCTTCAAAAGGGCTTT
AGGCAGAACTTGCAGGAAGTTGTTGCAGACCTACCCATCCTGAGACAGGGAGAAACTCTTGTAA
GTTGAATGCTCAGCACATTTGTATTGTCTGGACAGGGTCAGTGTCCTTCTGCTTAAAGATGACCT
ATGCTCCCAGACTCAGGCCCCTACCAGGGAGTCCCGGTCATTGCCAAAGAGCAGAACATCTGC
CTGTGCCTCAGGGCCTCACTTACCCTTGCCACAGGGACCTGGTGATACATCTCCACCTACTGGT
TGCTGAGAAAGTGAGTCACAGTCCACTTACTAGGGGTATTTGGCTTTTGGAGATGACTACTGGA
TGACATTAACTGTCTTGGGTTGCAGACAGAGGGAACCCAGCCAAAGAATCATCCTTTTCTCTATT
TCAGGGTACATCTATTGCTTTACATGCAGACAATCTTGTAATAATATATTCCCTAAAACATCAGTC
TCATGACAACAATCATATAAAGTGTGTATCCTCTCTTTGGCTTTGTAGATATCTAAGTTTACTGCT
TATCACGGTTAAGCTTAGAGTCATTCACTTCTGAGATTCTACTAAGATGAAAGTCATACACATTAA
GCTGTGTAGTTAGTCGCTGCACTTCTCTATACTGCCTCTTTCTATCCCTCTTGTTCAAGGATGGA
ATCTGGTATCTCCTCTTAGTCAGTGAAATCGGGGAGTAGCTGGTCCACTCAGCTCCTGAGGCAT
TTCTCATGCCAGATCTGGTTAGACAGCCCATTTGGGAGCCCTGCCTGCATGATGATAAGTGTTG
TCCTGCCCTGCTAGCACTCTGTTAACACCTTTCTCTCTGCCAAGGTCATGTTTCCTTTTTTATAGC
CCTGCCTTCATTAGATAAGTCAGACAGAGCCCAAGAGACAACTTCAGTATTTCTTAAAGACAAGA
GTTAGCACTTTGGAGATGCAAGCCGTGGAAGAGTGGGGCAAGATAATTAAGGATCCTGGACCT
CAAGGCAAATAAAGTGTGTCCCCAGCAGATCAAAACTGCTGTTTTCTGCAATCAACAATTAACCC
GCAATGATTTCGCTCCTGACATGGCCATCTTTGCTGTCTTCTGAACTTCCTGTAGGTGGCAGGT
CCTGGATGCTGTCCCATTTTGGTTCCAAGACTATTGTGTGGCTCTCTTGACAGGGGCCAGTCAA
GTGGCAGTTTTCTCCTCTGAGTTGCAGGAGGACAGGCTTAACAGGGTGGTTCATGTGGCGTATT
CCCACTTCTGCACTGTCTTCAAACAAAGTTGCAGTGTCCAGTAAAACAGCAGCCCAAATAGCAG
TGAGGTTAGATGTAGGTGGCCGGGCGGTGGGGGGGGGGGGTGCATTGGGAACTCTGGAACT
GCATGAATCCCACACGGCATACTGGATTTATAATAGAGTCACTCAGAGCTTCTCAAAGACCCTG
TTTTCAGGGAGGAAACAGACCAGGCTTTGTGCAGCTCCAGGCTGGGGTGCTGACATCACTGGA
CCCCTCCTTGTGGGGGGACGCTGTCTGTCACGTGAGCTGGGGCCATCAGTTTGAGCGTGGCTG
CTCCACAGGGCCCAAGATGGAGGCTTTTCTTCTCTTTCCAACACCGAAGGGCTGCATTGGTGGG
CCAGGGAGGGTGGCCTGACCCACCTGCATATCCAGTGCCCTTTAGCCTTCTTTGGGCTTCAAAT
GCAACCTTGGGGCACCCAGGGAAATGGAGCTTGCCTTTGATCATCTTCCCCCTCTGCAGCGTG
GCTTCCCAGTCATACTGTCGGCTGCATCCTTATTCCTCACTTTCTTTTTCCCTCTCCACACATTTC
TTTGTCTGTTTGGCAGTGCCCATAGGCATTGCTTGCTTCACCTGGTATGTGGCAACTCTGTTAGG
GGGACCATAGGCCCCTGTGGTCTGCATGGAGCTTAATCCTTTTACCATGGCGTGACTCCAATAA
TGGTAAAATGTCACTTCACTTCCATTGGCTTTAGGTAATGCCACCACTCACTTGTAGAGTGGCAT
ATAGATTTCCAGGGGTTTCCATACCCAACCACCCTTGGAAAGAGACAGGTAGGTTTTTCTTGCA
GCCTGAATGCCTGCTGGCCATGTAGTGTGGCCTGGGAATTGGTGACAGTGAAAGAAAGAGAAA
TAGGGCAGAAAATGTAAAGAAAACCCTCAGGTATTAAGGCCTAGGTGGCCAAGATTGGAAATAG
AGAGCAAGTTGTTTTCCTTGTTCCCATATTTTTGTGCTACCTCCTTTCAATTCTGGACCTGGAAGC
AGATTTCCTTCCAGAGGTAGAAATGTTCTTCCTACCCAGCAATTGTCAGCATCCGGGGTGGCGG
AGAGGGGGCCGCTCTGCCATGGACTCTCCGTCACCCAGCTTGTCCCTCTCCTTCCCCAGGTCT
GTGCGTGATGAGTGCTGCGGCCATCTACACGGTGAGGCACCCGGAGTGGCATCTCAACTCGGA
TTACTCCTACGGTTTCGCCTACATCCTGGCCTGGGTGGCCTTCCCCCTGGCCCTTCTCAGCGGT
GTCATCTATGTGATCTTGCGGAAACGCGAATGAGGCGCCCAGACGGTCTGTCTGAGGCTCTGA
GCGTACATAGGGAAGGGAGGAAGGGAAAACAGAAAGCAGACAAAGAAAAAAGAGCTAGCCCAA
AATCCCAAACTCAAACCAAACCAAACAGAAAGCAGTGGAGGTGGGGGTTGCTGTTGATTGAAGA
TGTATATAATATCTCCGGTTTATAAAACCTATTTATAACACTTTTTACATATATGTACATAGTATTG
TTTGCTTTTTATGTTGACCATCAGCCTCGTGTTGAGCCTTAAAGAAGTAGCTAAGGAACTTTACAT
CCTAACAGTATAATCCAGCTCAGTATTTTTGTTTTGTTTTTTGTTTGTTTGTTTTGTTTTACCCAGA
AATAAGATAACTCCATCTCGCCCCTTCCCTTTCATCTGAAAGAAGATACCTCCCTCCCAGTCCAC
CTCATTTAGAAAACCAAAGTGTGGGTAGAAACCCCAAATGTCCAAAAGCCCTTTTCTGGTGGGT
GACCCAGTGCATCCAACAGAAACAGCCGCTGCCCGAACCTCTGTGTGAAGCTTTACGCGCACA
CGGACAAAATGCCCAAACTGGAGCCCTTGCAAAAACACGGCTTGTGGCATTGGCATACTTGCCC
TTACAGGTGGAGTATCTTCGTCACACATCTAAATGAGAAATCAGTGACAACAAGTCTTTGAAATG
GTGCTATGGATTTACCATTCCTTATTATCACTAATCATCTAAACAACTCACTGGAAATCCAATTAA
CAATTTTACAACATAAGATAGAATGGAGACCTGAATAATTCTGTGTAATATAAATGGTTTATAACT
GCTTTTGTACCTAGCTAGGCTGCTATTATTACTATAATGAGTAAATCATAAAGCCTTCATCACTCC
CACATTTTTCTTACGGTCGGAGCATCAGAACAAGCGTCTAGACTCCTTGGGACCGTGAGTTCCT
AGAGCTTGGCTGGGTCTAGGCTGTTCTGTGCCTCCAAGGACTGTCTGGCAATGACTTGTATTGG
CCACCAACTGTAGATGTATATATGGTGCCCTTCTGATGCTAAGACTCCAGACCTTTTGTTTTTGC
TTTGCATTTTCTGATTTTATACCAACTGTGTGGACTAAGATGCATTAAAATAAACATCAGAGTAAC
TCACT

In certain embodiments, the antisense oligomer has sufficient length and complementarity to a sequence in exon 2 of the PMP22 pre-mRNA, exon 3 of the PMP22 pre-mRNA, exon 3 of the PMP22 pre-mRNA, exon 4 of the PMP22 pre-mRNA, or exon 5 of the PMP22 pre-mRNA. Also included are antisense oligomers which are complementary to a region that spans an exon 2/intron junction of the PMP22 pre-mRNA, a region that spans an exon 3/intron junction of the PMP22 pre-mRNA, a region that spans an exon 4/intron junction of the PMP22 pre-mRNA, or a region that spans an exon 5/intron junction of the PMP22 pre-mRNA. The exon 2 (SEQ ID NO: 2), exon 3 (SEQ ID NO: 3), exon 4 (SEQ ID NO: 4), or exon 5 (SEQ ID NO: 5) of the PMP22 gene (accession number NM_000304.4) are shown in Table 2B below.

TABLE 2B
Target sequences for PMP22 gene

| | | SEQ |
|---|---|---|
| | | ID |
| Name | Sequence (5′-3′) | NO |
| PMP- | GCAGAAACTCCGCTGAGCAGAACTTGCCGCCAGAATGCTCCTCCTG | 2 |
| 22 | TTGCTGAGTATCATCGTCCTCCACGTCGCGGTGCTGGTGCTGCTGT | |
| Exon2 | TCGTCTCCACGATCGTCAGC | |
| PMP- | CAATGGATCGTGGGCAATGGACACGCAACTGATCTCTGGCAGAACT | 3 |
| 22 | GTAGCACCTCTTCCTCAGGAAATGTCCACCACTGTTTCTCATCATCA | |
| Exon3 | CCAAACG | |
| PMP- | AATGGCTGCAGTCTGTCCAGGCCACCATGATCCTGTCGATCATCTT | 4 |
| 22 | CAGCATTCTGTCTCTGTTCCTGTTCTTCTGCCAACTCTTCACCCTCA | |
| Exon4 | CCAAGGGGGGCAGGTTTTACATCACTGGAATCTTCCAAATTCTTGCT | |
| | G | |
| PMP- | GTCTGTGCGTGATGAGTGCTGCGGCCATCTACACGGTGAGGCACC | 5 |
| 22 | CGGAGTGGCATCTCAACTCGGATTACTCCTACGGTTTCGCCTACAT | |
| Exon5 | CCTGGCCTGGGTGGCCTTCCCCCTGGCCCTTCTCAGCGGTGTCAT | |
| | CTATGTGATCTTGCGGAAACGCGAATGAGGCGCCCAGACGGTCTGT | |
| | CTGAGGCTCTGAGCGTACATAGGGAAGGGAGGAAGGGAAAACAGA | |
| | AAGCAGACAAAGAAAAAAGAGCTAGCCCAAAATCCCAAACTCAAAC | |
| | CAAACCAAACAGAAAGCAGTGGAGGTGGGGGTTGCTGTTGATTGAA | |
| | GATGTATATAATATCTCCGGTTTATAAAACCTATTTATAACACTTTTTA | |
| | CATATATGTACATAGTATTGTTTGCTTTTTATGTTGACCATCAGCCTC | |
| | GTGTTGAGCCTTAAAGAAGTAGCTAAGGAACTTTACATCCTAACAGT | |
| | ATAATCCAGCTCAGTATTTTTGTTTTGTTTTTTGTTTGTTTGTTTTGTT | |
| | TTACCCAGAAATAAGATAACTCCATCTCGCCCCTTCCCTTTCATCTG | |
| | AAAGAAGATACCTCCCTCCCAGTCCACCTCATTTAGAAAACCAAAGT | |
| | GTGGGTAGAAACCCCAAATGTCCAAAAGCCCTTTTCTGGTGGGTGA | |
| | CCCAGTGCATCCAACAGAAACAGCCGCTGCCCGAACCTCTGTGTGA | |
| | AGCTTTACGCGCACACGGACAAAATGCCCAAACTGGAGCCCTTGCA | |
| | AAAACACGGCTTGTGGCATTGGCATACTTGCCCTTACAGGTGGAGT | |
| | ATCTTCGTCACACATCTAAATGAGAAATCAGTGACAACAAGTCTTTG | |
| | AAATGGTGCTATGGATTTACCATTCCTTATTATCACTAATCATCTAAA | |
| | CAACTCACTGGAAATCCAATTAACAATTTTACAACATAAGATAGAATG | |
| | GAGACCTGAATAATTCTGTGTAATATAAATGGTTTATAACTGCTTTTG | |
| | TACCTAGCTAGGCTGCTATTATTACTATAATGAGTAAATCATAAAGCC | |
| | TTCATCACTCCCACATTTTTCTTACGGTCGGAGCATCAGAACAAGCG | |
| | TCTAGACTCCTTGGGACCGTGAGTTCCTAGAGCTTGGCTGGGTCTA | |
| | GGCTGTTCTGTGCCTCCAAGGACTGTCTGGCAATGACTTGTATTGG | |
| | CCACCAACTGTAGATGTATATATGGTGCCCTTCTGATGCTAAGACTC | |
| | CAGACCTTTTGTTTTTGCTTTGCATTTTCTGATTTTATACCAACTGTG | |
| | TGGACTAAGATGCATTAAAATAAACATCAGAGTAACTCA | |

In certain embodiments, antisense targeting sequences are designed to hybridize to a region of one or more of the target sequences listed in Tables 2A and 2B. Selected antisense targeting sequences can be made shorter, e.g., about 12 bases, or longer, e.g., about 40 bases, and include a small number of mismatches, as long as the sequence is sufficiently complementary to effect splice modulation upon hybridization to the target sequence, and optionally forms with the RNA a heteroduplex having a Tm of 45° C. or greater.

In certain embodiments, the degree of complementarity between the target sequence and antisense targeting sequence is sufficient to form a stable duplex. The region of complementarity of the antisense oligomers with the target RNA sequence may be as short as 8-11 bases, but can be 12-15 bases or more, e.g., 10-40 bases, 12-30 bases, 12-25 bases, 15-25 bases, 12-20 bases, or 15-20 bases, including all integers in between these ranges. An antisense oligomer of about 14-15 bases is generally long enough to have a unique complementary sequence. In certain embodiments, a minimum length of complementary bases may be required to achieve the requisite binding Tm, as discussed herein.

In certain embodiments, oligomers as long as 40 bases may be suitable, where at least a minimum number of bases, e.g., 10-12 bases, are complementary to the target sequence. In some embodiments, facilitated or active uptake in cells is optimized at oligomer lengths of less than about 30 bases. For PMO oligomers, described further herein, an optimum balance of binding stability and uptake generally occurs at lengths of 18-25 bases. Included in the disclosure are antisense oligomers (e.g., PMOs, PMO-X, PNAs, LNAs, 2′-OMe) that consist of about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 bases, in which at least about 6, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 contiguous or non-contiguous bases are complementary to the target sequences of Tables 2A and 2B.

The antisense oligomers typically comprises a base sequence which is sufficiently complementary to a sequence or region within or adjacent to exon 2, exon 3, exon 4, or exon 5 of the pre-mRNA sequence of the PMP22 gene. Ideally, an antisense oligomer is able to effectively modulate aberrant splicing of the PMP22 pre-mRNA, and thereby increase expression of active PMP22 protein. This requirement is optionally met when the oligomer compound has the ability to be actively taken up by mammalian cells, and once taken up, form a stable duplex (or heteroduplex) with the target mRNA, optionally with a Tm greater than about 40° C. or 45° C.

In certain embodiments, antisense oligomers may be 100% complementary to the target sequence, or may include mismatches, e.g., to accommodate variants, as long as a heteroduplex formed between the oligomer and target sequence is sufficiently stable to withstand the action of cellular nucleases and other modes of degradation which may occur in vivo. Hence, certain oligomers may have substantial complementarity, meaning, about or at least about 70% sequence complementarity, e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence complementarity, between the oligomer and the target sequence. Oligomer backbones that are less susceptible to cleavage by nucleases are discussed herein. Mismatches, if present, are typically less destabilizing toward the end regions of the hybrid duplex than in the middle. The number of mismatches allowed will depend on the length of the oligomer, the percentage of G:C base pairs in the duplex, and the position of the mismatch(es) in the duplex, according to well understood principles of duplex stability. Although such an antisense oligomer is not necessarily 100% complementary to the v target sequence, it is effective to stably and specifically bind to the target sequence, such that splicing of the target pre-RNA is modulated.

The stability of the duplex formed between an oligomer and a target sequence is a function of the binding Tm and the susceptibility of the duplex to cellular enzymatic cleavage. The Tm of an oligomer with respect to complementary-sequence RNA may be measured by conventional methods, such as those described by Hames et al., Nucleic Acid Hybridization, IRL Press, 1985, pp. 107-108 or as described in Miyada C. G. and Wallace R. B., 1987, Oligomer Hybridization Techniques, Methods Enzymol. Vol. 154 pp. 94-107. In certain embodiments, antisense oligomers may have a binding Tm, with respect to a complementary-sequence RNA, of greater than body temperature and preferably greater than about 45° C. or 50° C. Tm's in the range 60-80° C. or greater are also included. According to well-known principles, the Tm of an oligomer, with respect to a complementary-based RNA hybrid, can be increased by increasing the ratio of C:G paired bases in the duplex, and/or by increasing the length (in base pairs) of the heteroduplex. At the same time, for purposes of optimizing cellular uptake, it may be advantageous to limit the size of the oligomer. For this reason, compounds that show high Tm (45-50° C. or greater) at a length of 25 bases or less are generally preferred over those requiring greater than 25 bases for high Tm values.

TABLE 3
below shows exemplary targeting sequences (in a 5′-to-3′ orientation)
complementary to pre-mRNA sequences of the PMP22 gene.
Antisense oligomer sequences for PMP22-targeted oligomers

| Sequence (5′-3′) | Gene Location | SEQ ID NO |
|---|---|---|
| CTGCGAGGAGAGCGCTGGGCGTGAG | PMP22 H2A (−25−1) | 6 |
| AAGTTCTGCTCAGCGGAGTTTCTGC | PMP22 H2A (+1+25) | 7 |
| CAACAGGAGGAGCATTCTGGCGGCA | PMP22 H2A (+25+49) | 8 |
| CTCAGCAACAGGAGGAGCATTCTGG | PMP22 H2A (+30+54) | 9 |
| TGATACTCAGCAACAGGAGGAGCAT | PMP22 H2A (+35+59) | 10 |
| ATACTCAGCAACAGGAGGAG | PMP22 H2A (+38+57) | 11 |
| TGATACTCAGCAACAGGAGG | PMP22 H2A (+40+59) | 12 |
| GACGATGATACTCAGCAACAGGAGG | PMP22 H2A (+40+64) | 13 |
| GATGATACTCAGCAACAGGA | PMP22 H2A (+42+61) | 14 |
| ACGATGATACTCAGCAACAG | PMP22 H2A (+44+63) | 15 |
| TGGAGGACGATGATACTCAGCAACA | PMP22 H2A (+45+69) | 16 |
| GGACGATGATACTCAGCAAC | PMP22 H2A (+46+65) | 17 |
| GAGGACGATGATACTCAGCA | PMP22 H2A (+48+67) | 18 |
| TGGAGGACGATGATACTCAG | PMP22 H2A (+50+69) | 19 |
| CGACGTGGAGGACGATGATACTCAG | PMP22 H2A (+50+74) | 20 |
| CGTGGAGGACGATGATACTC | PMP22 H2A (+52+71) | 21 |
| GACGTGGAGGACGATGATAC | PMP22 H2A (+54+73) | 22 |
| CACCGCGACGTGGAGGACGATGATA | PMP22 H2A (+55+79) | 23 |
| GCGACGTGGAGGACGATGAT | PMP22 H2A (+56+75) | 24 |
| ACCAGCACCGCGACGTGGAGGACGA | PMP22 H2A (+60+84) | 25 |
| GCAGCACCAGCACCGCGACGTGGAG | PMP22 H2A (+65+89) | 26 |
| GAACAGCAGCACCAGCACCGCGACG | PMP22 H2A (+70+94) | 27 |
| GAGACGAACAGCAGCACCAGCACCG | PMP22 H2A (+75+99) | 28 |
| AGGCACTCACGCTGACGATCGTGGA | PMP22 H2D (+15−10) | 29 |
| CGATCCATTGCTAGAGAGAATCAGA | PMP22 H3A (−15+10) | 30 |
| CGTGTCCATTGCCCACGATCCATTG | PMP22 H3A (+1+25) | 31 |
| CCAGAGATCAGTTGCGTGTCCATTG | PMP22 H3A (+15+39) | 32 |
| ACAGTTCTGCCAGAGATCAGTTGCG | PMP22 H3A (+24+48) | 33 |
| GACATTTCCTGAGGAAGAGGTGCTA | PMP22 H3A (+48+72) | 34 |
| GATGAGAAACAGTGGTGGACATTTC | PMP22 H3A (+65+89) | 35 |
| TTTGGTGATGATGAGAAACAGTGGT | PMP22 H3A (+74+98) | 36 |
| AGCCTCACCGTTTGGTGATGATGAG | PMP22 H3D (+17−8) | 37 |
| CACCGTTTGGTGATGATGAGAAACA | PMP22 H3D (+22-3) | 38 |
| CAGACTGCAGCCATTCTGGGGGAAA | PMP22 H4A (−10+15) | 39 |
| GAATGCTGAAGATGATCGACAGGAT | PMP22 H4A (+30+54) | 40 |
| AGAGTTGGCAGAAGAACAGGAACAG | PMP22 H4A (+60+84) | 41 |
| TGTAAAACCTGCCCCCCTTGGTGAG | PMP22 H4A (+90+114) | 42 |
| ATTCCAGTGATGTAAAACCTGCCCC | PMP22 H4A | 43 |
| | (+100+124) | |
| AATTTGGAAGATTCCAGTGATGTAA | PMP22 H4A | 44 |
| | (+110+134) | |
| TACCAGCAAGAATTTGGAAGATTCC | PMP22 H4D (+22−3) | 45 |
| CACTCATCACGCACAGACCTGGGGAA | PMP22 H5A (−8+17) | 46 |
| GCCTCACCGTGTAGATGGCCGCAGC | PMP22 H5A (+18+42) | 47 |
| TTGAGATGCCACTCCGGGTGCCTCA | PMP22 H5A (+37+61) | 48 |
| CCGTAGGAGTAATCCGAGTTGAGAT | PMP22 H5A (+55+79) | 49 |
| CTCTGATGTTTATTTTAATGCATCT | PMP22 H5A | 50 |
| | (+1271+1295) | |

For any of the sequences in Table 3, any of the antisense oligomer chemistries disclosed herein or any of the antisense oligomer carrier peptide conjugates disclosed herein may be used.

Certain antisense oligomers thus comprise, consist, or consist essentially of, a sequence in Table 3 (e.g., SEQ ID NOs: 6-50) or a variant or contiguous or non-contiguous portion(s) thereof. For instance, certain antisense oligomers comprise about or at least about 6, 7, 8, 9, 10, 11, 12, 13, 14, 15, 16, 17, 18, 19, 20, 21, 22, 23, 24, 25, 26, or 27 contiguous or non-contiguous nucleotides of any of SEQ ID NOs: 6-50. For non-contiguous portions, intervening nucleotides can be deleted or substituted with a different nucleotide, or intervening nucleotides can be added. Additional examples of variants include oligomers having about or at least about 70% sequence identity or homology, e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity or homology, over the entire length of any of SEQ ID NOs: 6-50. In some embodiments, the antisense oligomer or compound with a targeting sequence that comprises, consists of, or consists essentially of such a variant sequence increases, enhances, or promotes exon 2, exon 3, exon 4, or exon 5 exclusion in the PMP22 mRNA, optionally, by at least about 10%, 15%, 20%, 25%, 30%, 35%, 40%, 45%, 50%, 55%, 60%, or 65% or more relative to a control, according to at least one of the examples or methods described herein. In some embodiments, the antisense oligomer or compound with a targeting sequence that comprises, consists of, or consists essentially of such a variant sequence reduces PMP22 protein expression in a cell, optionally, by at least about 10%, 15%, 20%, 25%, 30%, 35%, 40%, 45%, 50%, 55%, 60%, or 65% or more relative to a control, according to at least one of the examples or methods described herein. In some embodiments, the antisense oligomer or compound comprising, consisting of, or consisting essentially of such a variant sequence reduces PMP22 activity in a cell, optionally, by at least about 10%, 15%, 20%, 25%, 30%, 35%, 40%, 45%, 50%, 55%, 60%, or 65% or more relative to a control, according to at least one of the examples or methods described herein.

In various aspects an antisense oligomer or compound is provided, comprising a targeting sequence that is complementary (e.g., at least 80, 81, 82, 83, 84, 85, 86, 87, 88, 89, 90, 91, 92, 93, 94, 95, 96, 97, 98, 99, or 100% complementary) to a target region of the PMP22 pre-mRNA, optionally where the targeting sequences is as set forth in Table 3. In another aspect, an antisense oligomer or compound is provided, comprising a variant targeting sequence, such as any of those described herein, wherein the variant targeting sequence binds to a target region of the PMP22 pre-mRNA that is complementary (e.g., 80%-100% complementary) to one or more of the targeting sequences set forth in Table 3. In some embodiments, the antisense oligomer or compound binds to a target sequence comprising at least 10 (e.g., at least 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40) consecutive bases of the PMP22 pre-mRNA (e.g., any of SEQ ID NOs: 2, 3, 4, or 5 or a sequence that spans a PMP22 pre-mRNA splice junction defined by SEQ ID NO: 2 and an intron preceding or proceeding SEQ ID NO: 2, SEQ ID NO: 3 and an intron preceding or proceeding SEQ ID NO: 3, SEQ ID NO: 4 and an intron preceding or proceeding SEQ ID NO: 4, or SEQ ID NO: 5 and an intron preceding or proceeding SEQ ID NO: 5). In some embodiments, the target sequence is complementary (e.g., at least 80, 81, 82, 83, 84, 85, 86, 87, 88, 89, 90, 91, 92, 93, 94, 95, 96, 97, 98, 99, or 100% complementary) to one or more of the targeting sequences set forth in Table 3. In some embodiments, the target sequence is complementary (e.g., at least 80, 81, 82, 83, 84, 85, 86, 87, 88, 89, 90, 91, 92, 93, 94, 95, 96, 97, 98, 99, or 100% complementary) to at least 10 (e.g., at least 11, 12, 13, 14, 15, 16, 17, 18, 19, 20, 21, 22, 23, 24, 25, 26, 27, or 28) consecutive bases of one or more of the targeting sequences set forth in Table 3.

The activity of antisense oligomers and variants thereof can be assayed according to routine techniques in the art. For example, splice forms and expression levels of surveyed RNAs and proteins may be assessed by any of a wide variety of well-known methods for detecting splice forms and/or expression of a transcribed nucleic acid or protein. Non-limiting examples of such methods include RT-PCR of spliced forms of RNA followed by size separation of PCR products, nucleic acid hybridization methods e.g., Northern blots and/or use of nucleic acid arrays; nucleic acid amplification methods; immunological methods for detection of proteins; protein purification methods; and protein function or activity assays.

RNA expression levels can be assessed by preparing mRNA/cDNA (i.e., a transcribed polynucleotide) from a cell, tissue or organism, and by hybridizing the mRNA/cDNA with a reference polynucleotide that is a complement of the assayed nucleic acid, or a fragment thereof. cDNA can, optionally, be amplified using any of a variety of polymerase chain reaction or in vitro transcription methods prior to hybridization with the complementary polynucleotide; preferably, it is not amplified. Expression of one or more transcripts can also be detected using quantitative PCR to assess the level of expression of the transcript(s).

### IV. Peptide Transporters

In some embodiments, the subject oligomer is conjugated to a peptide transporter moiety, for example a cell-penetrating peptide transport moiety (also referred to as a cell-penetrating peptide), which is effective to enhance transport of the oligomer into cells. For example, in some embodiments the peptide transporter moiety is an arginine-rich peptide. In further embodiments, the transport moiety is attached to either the 5′ or 3′ terminus of the oligomer. When such peptide is conjugated to either terminus, the opposite terminus is then available for further conjugation to a modified terminal group as described herein.

In some embodiments of the foregoing, the peptide transport moiety comprises 6 to 16 subunits selected from X′ subunits, Y′ subunits, and Z′ subunits,
where
(a) each X′ subunit independently represents lysine, arginine, or an arginine analog, said analog being a cationic-amino acid comprising a side chain of the structure R³³N═C(NH₂)R³⁴, where R³³ is H or R; R³⁴ is R³⁵, NH₂, NHR, or NR³⁴, where R³⁵ is lower alkyl or lower alkenyl and may further include oxygen or nitrogen; R³³ and R³⁴ may together form a ring; and the side chain is linked to said amino acid via R³³ or R³⁴;
(b) each Y′ subunit independently represents a neutral amino acid —C(O)—(CHR)ₙ—NH—, where n is 2 to 7 and each R is independently H or methyl; and
(c) each Z′ subunit independently represents an α-amino acid having a neutral aralkyl side chain;
wherein the peptide comprises a sequence represented by one of (X′Y′X′)ₚ, (X′Y′)ₘ, and (X′Z′Z′)ₚ, where p is 2 to 5 and m is 2 to 8.

In selected embodiments, for each X′, the side chain moiety is guanidyl, as in the amino acid subunit arginine (Arg). In further embodiments, each Y′ is —CO—(CH₂)ₙ—CHR—NH—, where n is 2 to 7 and R is H. For example, when n is 5 and R is H, Y′ is a 6-aminohexanoic acid subunit, abbreviated herein as Ahx (or simply X); when n is 2 and R is H, Y′ is a β-alanine subunit (referred to herein as B).

In certain embodiments, peptides of this type include those comprising arginine dimers alternating with single Y′ subunits, where Y′ is Ahx. Examples include peptides having the formula (RY′R)ₚ or the formula (RRY′)ₚ, where Y′ is Ahx. In one embodiment, Y′ is a 6-aminohexanoic acid subunit, R is arginine, and p is 4.

In a further embodiment, each Z′ is phenylalanine, and m is 3 or 4.

In some embodiments, the conjugated peptide is linked to a terminus of the oligomer via a linker Ahx-B, where Ahx is a 6-aminohexanoic acid subunit and B is a β-alanine subunit.

In selected embodiments, for each X′, the side chain moiety is independently selected from the group consisting of guanidyl (HN═C(NH₂)NH—), amidinyl (HN═C(NH₂)C—), 2-aminodihydropyrimidyl, 2-aminotetrahydropyrimidyl, 2-aminopyridinyl, and 2-aminopyrimidonyl, and it is preferably selected from guanidyl and amidinyl. In one embodiment, the side chain moiety is guanidyl, as in the amino acid subunit arginine (Arg (R)).

In some embodiments, the Y′ subunits are either contiguous, in that no X′ subunits intervene between Y′ subunits, or interspersed singly between X′ subunits. However, in some embodiments the linking subunit may be between Y′ subunits. In one embodiment, the Y′ subunits are at a terminus of the peptide transporter; in other embodiments, they are flanked by X′ subunits. In further embodiments, each Y′ is —CO—(CH₂)ₙ—CHR—NH—, where n is 2 to 7 and R is H. For example, when n is 5 and R is H, Y′ is a 6-aminohexanoic acid subunit, abbreviated herein as Ahx. In selected embodiments of this group, each X′ comprises a guanidyl side chain moiety, as in an arginine subunit. Exemplary peptides of this type include those comprising arginine dimers alternating with single Y′ subunits, where Y′ is preferably Ahx. Examples include peptides having the formula (RY′R)₄ or the formula (RRY′)₄ (SEQ ID NO: 72), where Y′ is preferably Ahx. In some embodiments, the nucleic acid analog is linked to a terminal Y′ subunit, preferably at the C-terminus. In other embodiments, the linker is of the structure AhxB, where Ahx is a 6-aminohexanoic acid subunit and B is a β-alanine subunit.

The peptide transport moieties as described above have been shown to greatly enhance cell entry of attached oligomers, relative to uptake of the oligomer in the absence of the attached transport moiety, and relative to uptake by an attached transport moiety lacking the hydrophobic subunits Y′. Such enhanced uptake may be evidenced by at least a two-fold increase, or in other embodiments a four-fold increase, in the uptake of the compound into mammalian cells relative to uptake of the agent by an attached transport moiety lacking the hydrophobic subunits Y′. In some embodiments, uptake is enhanced at least twenty-fold or at least forty-fold, relative to the unconjugated compound.

A further benefit of the peptide transport moiety is its expected ability to stabilize a duplex between an antisense oligomer and its target nucleic acid sequence. While not wishing to be bound by theory, this ability to stabilize a duplex may result from the electrostatic interaction between the positively charged transport moiety and the negatively charged nucleic acid. In some embodiments, the number of charged subunits in the transporter is less than 14, or in other embodiments between 8 and 11, since too high a number of charged subunits may lead to a reduction in sequence specificity.

Exemplary arginine-rich cell-penetrating peptide transporters are given below in Table 4.

TABLE 4
Arginine-Rich Cell-Penetrating Peptide
Transporters

| Name (Designation) | Sequence | SEQ ID NO |
|---|---|---|
| rTAT | RRRQRRKKR | 51 |
| Tat | RKKRRQRRR | 52 |
| R₉F₂ | RRRRRRRRRFF | 53 |
| R₅F₂R₄ | RRRRRFFRRRR | 54 |
| R₄ | RRRR | 55 |
| R₅ | RRRRR | 56 |
| R₆ | RRRRRR | 57 |
| R₇ | RRRRRRR | 58 |
| R₈ | RRRRRRRR | 59 |
| R₉ | RRRRRRRRR | 60 |
| (RXR)₄ | RXRRXRRXRRXR | 61 |
| (RXR)₅ | RXRRXRRXRRXRRXR | 62 |
| (RXRRBR)₂ | RXRRBRRXRRBR | 63 |
| (RAR)₄F₂ | RARRARRARRARFF | 64 |
| (RGR)₄F₂ | RGRRGRRGRRGRFF | 65 |

^{a}Sequences assigned to SEQ ID NOs do not include the linkage portion (e.g., proline and glycine). X and B refer to 6-aminohexanoic acid and beta-alanine, respectively.

### V. Pharmaceutical Compositions

The present disclosure also provides for formulation and delivery of the disclosed oligomers. Accordingly, an aspect of the present disclosure is a pharmaceutical composition comprising an antisense compound as disclosed herein and a pharmaceutically acceptable carrier.

Effective delivery of the antisense oligomer to the target nucleic acid is an important aspect of treatment. Routes of antisense oligomer delivery include, but are not limited to, various systemic routes, including oral and parenteral routes, e.g., intravenous, subcutaneous, intraperitoneal, and intramuscular, as well as inhalation, transdermal and topical delivery. The appropriate route may be determined by one of skill in the art, as appropriate to the condition of the subject under treatment. For example, an appropriate route for delivery of an antisense oligomer in the treatment of a viral infection of the skin is topical delivery, while delivery of an antisense oligomer for the treatment of a viral respiratory infection can be intravenous or by inhalation. The oligomer may also be delivered directly to any particular site of viral infection.

The antisense oligomer can be administered in any convenient vehicle which is physiologically and/or pharmaceutically acceptable. Such a composition can include any of a variety of standard pharmaceutically acceptable carriers employed by those of ordinary skill in the art. Examples include, but are not limited to, saline, phosphate buffered saline (PBS), water (e.g., sterile water for injection), aqueous ethanol, emulsions such as oil/water emulsions or triglyceride emulsions, tablets and capsules. The choice of suitable physiologically acceptable carrier will vary dependent upon the chosen mode of administration.

The instant compounds (e.g., oligomers) can generally be utilized as the free acid or free base. Alternatively, the instant compounds may be used in the form of acid or base addition salts. Acid addition salts of the free amino compounds may be prepared by methods well known in the art, and may be formed from organic and inorganic acids. Suitable organic acids include maleic, fumaric, benzoic, ascorbic, succinic, methanesulfonic, acetic, trifluoroacetic, oxalic, propionic, tartaric, salicylic, citric, gluconic, lactic, mandelic, cinnamic, aspartic, stearic, palmitic, glycolic, glutamic, and benzenesulfonic acids. Suitable inorganic acids include hydrochloric, hydrobromic, sulfuric, phosphoric, and nitric acids. Base addition salts included those salts that form with the carboxylate anion and include salts formed with organic and inorganic cations such as those chosen from the alkali and alkaline earth metals (for example, lithium, sodium, potassium, magnesium, barium and calcium), as well as the ammonium ion and substituted derivatives thereof (for example, dibenzylammonium, benzylammonium, 2-hydroxyethylammonium, and the like). Thus, the term “pharmaceutically acceptable salt” of structure (I) is intended to encompass any and all acceptable salt forms.

In addition, prodrugs are also included within the context of this invention. Prodrugs are any covalently bonded carriers that release a compound of structure (I) in vivo when such prodrug is administered to a patient. Prodrugs are generally prepared by modifying functional groups in a way such that the modification is cleaved, either by routine manipulation or in vivo, yielding the parent compound. Prodrugs include, for example, compounds of this invention wherein hydroxy, amine or sulfhydryl groups are bonded to any group that, when administered to a patient, cleaves to form the hydroxy, amine or sulfhydryl groups. Thus, representative examples of prodrugs include (but are not limited to) acetate, formate and benzoate derivatives of alcohol and amine functional groups of the compounds of structure (I). Further, in the case of a carboxylic acid (—COOH), esters may be employed, such as methyl esters, ethyl esters, and the like.

In some instances, liposomes may be employed to facilitate uptake of the antisense oligonucleotide into cells. (See, e.g., Williams, S. A., Leukemia 10(12): 1980-1989, 1996; Lappalainen et al. (1994) Antiviral Res. 23:119; Uhlmann et al. (1990) Antisense Oligonucleotides: A New Therapeutic Principle, Chemical Reviews, Volume 90, No. 4, pages 544-584; Gregoriadis, G., Chapter 14, Liposomes, Drug Carriers in Biology and Medicine, pp. 287-341, Academic Press, 1979). Hydrogels may also be used as vehicles for antisense oligomer administration, for example, as described in WO 93/01286. Alternatively, the oligonucleotides may be administered in microspheres or microparticles. (See, e.g., Wu, GY and Wu C H (1987) J Biol Chem. 262:4429-4432). Alternatively, the use of gas-filled microbubbles complexed with the antisense oligomers can enhance delivery to target tissues, as described in U.S. Pat. No. 6,245,747. Sustained release compositions may also be used. These may include semipermeable polymeric matrices in the form of shaped articles such as films or microcapsules.

### VI. Methods of Making

Preparation of Oligomers with Basic Nitrogen Internucleoside Linkers

Morpholino subunits, the modified intersubunit linkages, and oligomers comprising the same can be prepared as described, for example, in U.S. Pat. Nos. 5,185,444, and 7,943,762, which are incorporated by reference in their entireties. The morpholino subunits can be prepared according to the following general Reaction Scheme 1.

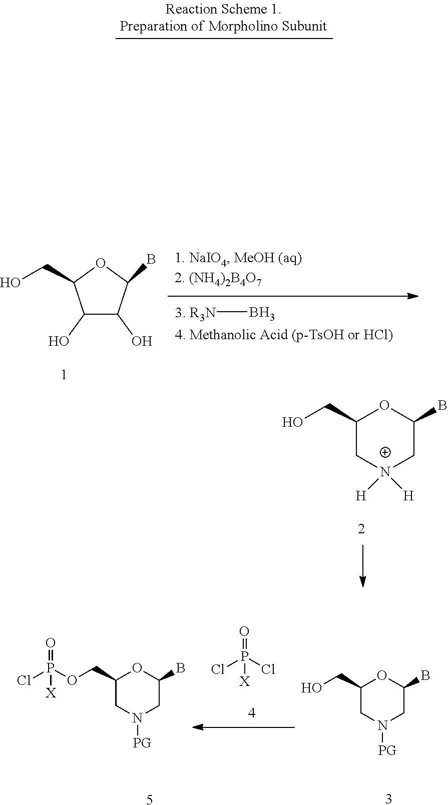

Referring to Reaction Scheme 1, wherein B represents a base pairing moiety and PG represents a protecting group, the morpholino subunits may be prepared from the corresponding ribonucleoside (1) as shown. The morpholino subunit (2) may be optionally protected by reaction with a suitable protecting group precursor, for example trityl chloride. The 3′ protecting group is generally removed during solid-state oligomer synthesis as described in more detail below. The base pairing moiety may be suitably protected for sold phase oligomer synthesis. Suitable protecting groups include benzoyl for adenine and cytosine, phenylacetyl for guanine, and pivaloyloxymethyl for hypoxanthine (I). The pivaloyloxymethyl group can be introduced onto the N1 position of the hypoxanthine heterocyclic base. Although an unprotected hypoxanthine subunit, may be employed, yields in activation reactions are far superior when the base is protected. Other suitable protecting groups include those disclosed in U.S. Pat. No. 8,076,476, which is hereby incorporated by reference in its entirety.

Reaction of 3 with the activated phosphorous compound 4 results in morpholino subunits having the desired linkage moiety 5. Compounds of structure 4 can be prepared using any number of methods known to those of skill in the art. For example, such compounds may be prepared by reaction of the corresponding amine and phosphorous oxychloride. In this regard, the amine starting material can be prepared using any method known in the art, for example those methods described in the Examples and in U.S. Pat. No. 7,943,762.

Compounds of structure 5 can be used in solid-phase automated oligomer synthesis for preparation of oligomers comprising the intersubunit linkages. Such methods are well known in the art. Briefly, a compound of structure 5 may be modified at the 5′ end to contain a linker to a solid support. For example, compound 5 may be linked to a solid support by a linker. Once supported, the protecting group (e.g., trityl) is removed and the free amine is reacted with an activated phosphorous moiety of a second compound of structure 5. This sequence is repeated until the desired length of oligo is obtained. The protecting group in the terminal 3′ end may either be removed or left on if a 3′-modification is desired.

The preparation of modified morpholino subunits and morpholino oligomers are described in more detail in the Examples. The morpholino oligomers containing any number of modified linkages may be prepared using methods described herein, methods known in the art and/or described by reference herein. Also described in the examples are global modifications of morpholino oligomers prepared as previously described (see e.g., PCT publication WO 2008/036127).

Synthesis of PMO, PMO+, PPMO, and PMO-X containing further linkage modifications as described herein was done using methods known in the art and described in pending U.S. Pat. Nos. 8,299,206 and 8,076,476 and PCT publication numbers WO 2009/064471, WO 2011/150408 and WO 2012/150960, which are hereby incorporated by reference in their entirety.

PMO with a 3′ trityl modification are synthesized essentially as described in PCT publication number WO 2009/064471 with the exception that the detritylation step is omitted.

### VII. Methods of Treatment

Provided herein is a method of treating a disease associated with dysregulation of peripheral myelin protein 22. The method comprises administering to a patient in need thereof a therapeutically effective amount of an antisense compound disclosed herein, or a pharmaceutical composition thereof. In an embodiment, the disease associated with dysregulation of peripheral myelin protein 22 is Charcot-Marie-Tooth type 1A (CMT1A).

In certain embodiments, the method is an in vitro method. In certain other embodiments, the method is an in vivo method.

In certain embodiments, the host cell is a mammalian cell. In certain embodiments, the host cell is a non-human primate cell. In certain embodiments, the host cell is a human cell.

In certain embodiments, the host cell is a naturally occurring cell. In certain other embodiments, the host cell is an engineered cell.

In certain embodiments, the antisense compound is administered to a mammalian subject, e.g., a human or a laboratory or domestic animal, in a suitable pharmaceutical carrier.

In certain embodiments, the antisense compound is administered to a mammalian subject, e.g., a human or laboratory or domestic animal, together with an additional agent. The antisense compound and the additional agent can be administered simultaneously or sequentially, via the same or different routes and/or sites of administration. In certain embodiments, the antisense compound and the additional agent can be co-formulated and administered together. In certain embodiments, the antisense compound and the additional agent can be provided together in a kit.

In one embodiment, the oligomer is a phosphorodiamidate morpholino oligomer, contained in a pharmaceutically acceptable carrier, and is delivered intramuscularly. In another embodiment, the oligomer is a peptide-conjugated phosphorodiamidate morpholino oligomer, contained in a pharmaceutically acceptable carrier, and is delivered intramuscularly.

In another embodiment, the oligomer is a phosphorodiamidate morpholino oligomer, contained in a pharmaceutically acceptable carrier, and is delivered intravenously (i.v.). In another embodiment, the oligomer is a peptide-conjugated phosphorodiamidate morpholino oligomer, contained in a pharmaceutically acceptable carrier, and is delivered intravenously.

Additional routes of administration, e.g., oral, subcutaneous, intraperitoneal, and pulmonary, are also contemplated by the instant disclosure.

An effective in vivo treatment regimen using the antisense oligonucleotides may vary according to the duration, dose, frequency, and route of administration, as well as the condition of the subject under treatment (i.e., prophylactic administration versus administration in response to localized or systemic infection). Accordingly, such in vivo therapy will often require monitoring by tests under treatment, and corresponding adjustments in the dose or treatment regimen, in order to achieve an optimal therapeutic outcome.

In some embodiments, the oligomer is actively taken up by mammalian cells. In further embodiments, the oligomer can be conjugated to a transport moiety (e.g., transport peptide) as described herein to facilitate such uptake.

Also provided herein is a method of reducing peripheral myelin protein 22 expression in a patient in need thereof, comprising administering a therapeutically effective amount of the antisense oligomer disclosed herein.

In an embodiment, the patient has a disease associated with dysregulation of peripheral myelin protein 22 in a subject in need thereof. In a further embodiment, the patient has Charcot-Marie-Tooth type 1A.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### EXAMPLES

### Example 1—Oligomer Synthesis

Each synthesis column was filled with 30+/−2 mg of functionalized amino methyl polystyrene resin at 1% DVB cross linking. The oligomer is built on the resin with a cleavable disulfide (DSA) or Nitrocarboxyphenylpropyl (NCP2) anchor which allows for oligomer isolation from the resin and further purification and modification. Depending on the need, the resin may also have a polyethylene glycol tail spacer. The resin loads range from 325 to 475 μmol/g depending on the need. Therefore, each synthesis column has a maximal yield of 12 μmol. These amounts are typically enough for biological high throughput screening. When more material is required the process is transferred to the large scale internal production team for scale up.

For the example, a DSA loaded resin was loaded with a starting load of 342.6 μmol/g.

TABLE 5
Table of Synthesis cycle information

| Step | Volume | Delivery | Hold time |
|---|---|---|---|
| Detritylation Solution | 1.5 mL | Manifold | 15 seconds |
| Detritylation Solution | 1.5 mL | Manifold | 15 seconds |
| Detritylation Solution | 1.5 mL | Manifold | 15 seconds |
| Detritylation Solution | 1.5 mL | Manifold | 15 seconds |
| Detritylation Solution | 1.5 mL | Manifold | 15 seconds |
| Detritylation Solution | 1.5 mL | Manifold | 15 seconds |
| Detritylation Solution | 1.5 mL | Manifold | 15 seconds |
| DCM | 1.5 mL | Manifold | 30 seconds |
| Neutralization Solution | 1.5 mL | Manifold | 30 seconds |
| Neutralization Solution | 1.5 mL | Manifold | 30 seconds |
| Neutralization Solution | 1.5 mL | Manifold | 30 seconds |
| Neutralization Solution | 1.5 mL | Manifold | 30 seconds |
| Neutralization Solution | 1.5 mL | Manifold | 30 seconds |
| Neutralization Solution | 1.5 mL | Manifold | 30 seconds |
| DCM | 1.5 mL | Manifold | 30 seconds |
| Coupling | 350 uL-500 uL | Syringe Manifold | 40 minutes |
| DCM | 1.5 mL | Manifold | 30 seconds |
| Neutralization Solution | 1.5 mL | Manifold | 30 seconds |
| Neutralization Solution | 1.5 mL | Manifold | 30 seconds |
| DCM | 1.5 mL | Manifold | 30 seconds |
| DCM | 1.5 mL | Manifold | 30 seconds |
| DCM | 1.5 mL | Manifold | 30 seconds |

The starting amount of coupling solution for a 30 mg column is 350 μL. A 2% increase in coupling solution is used for each sequential base to maintain coupling solution coverage over the resin bed.

After synthesis was complete, the oligomer was cleaved off the resin. The protecting groups were removed from the heterocyclic bases prior to purification using the DTT cleavage solution (0.1 M Dithiothreitol in 10% Triethylamine/NMP, 25° C., at 53 mL/g starting resin). After the 30-minute incubation, the solution was filtered into 12 mL scintillation vials and the resin rinsed with additional cleavage solution. The cleaved PMO's were then diluted 2× with concentrated ammonium hydroxide, sealed tightly, and incubated at 45° C. in an oven for 16 to 18 hours. After incubation, the solution was cooled to room temperature. If continuing, the oligomers were purified by Strong Anion Exchange (SAX) purification, the sample was diluted 4× with Buffer A (1% Ammonium hydroxide) and purified using Macro-Prep High Q support Resin on a BioRad LP 10 (both from Bio-Rad, Hercules, CA).

If the sample is purified later, or is only undergoing crude isolation, it was isolated by Solid Phase Extraction (SPE) (Amberchrom CG300M, Dow Chemicals, MI), and diluted 20× with 1% NH₄OH in water. Once loaded, the product was washed 3×8 mL with 1% Ammonium hydroxide, and then eluted using 2×3 mL 45% Acetonitrile into a clean scintillation vail. The sample was then frozen and lyophilized down to dryness for at least two days.

### Example 2.—Strong Anion Exchange (SAX) Purification of PMO

PMO samples were purified using a SAX gradient with 1 M Sodium chloride in 1% Ammonium hydroxide as Buffer B. The gradient amount of Buffer B is dependent on the percentage of guanine and thymine bases in the sequence. Targeting the main peak near 30 minutes of the run, a gradient of X=40, 60, 80, or 100% Buffer B was selected (See Table 6 below). The purification was run at a flow rate of 7 mL/min with fractions being collected every minute (7 mL per fraction). Using a 50 mL column, the 60 min gradient elution was over ˜9 Column Volumes (CV) with fractions being selected and pooled based on UV absorbance.

TABLE 6
Table of SAX purification gradient

| Time | % A | % B |
|---|---|---|
| 0 | 100 | 0 |
| 0 | 100 | 0 |
| 60 | 100-X | X |
| 65 | 100-X | X |
| 66 | 100 | 0 |
| 75 | 100 | 0 |

Buffer A: 1% NH₄OH
Buffer B: 1% NH₄OH/1M NaCl

For the example, the gradient is run up to 60% Buffer B over a 60 minute linear gradient.

Pooled fractions were diluted by adding a five-fold volumetric excess of water. The conjugate/salt solution was then loaded onto a SPE column (Amberchrom CG300M, Dow Chemicals, MI, SP20SS Seprabeads, Sorbent Technologies, Norcross GA), which is subsequently washed with 3×8 mL-of 1% Ammonium Hydroxide to remove salt. Finally, the oligomer was eluted off from the SPE column with 2×3 mL of 45% Acetonitrile, then the oligomer was lyophilized down to dryness for two days. The sample was then resuspended in a known amount of 1% Ammonium Hydroxide and diluted 500× into a 1 mL cuvette. The OD absorbance was measured at 260 nm on a Cary 100 UV-Vis Spectrophotometer (Agilent, Wilmington, DE).

### Example 3.—Peptide Conjugation

A 1.00 equivalent of the PMO from Example 2 was combined with 1.25 equivalents of cell penetrating peptide (CPP), and 1.875 equivalents of DIPEA as the base and 1.875 equivalents of TBTU as a coupling reagent, resulting in deprotonation of the C-terminal end of the peptide allowing it to be activated by the coupling reagent. This activated CPP intermediate then reacts with the morpholine amine on the 3′ end of the oligonucleotide to form an amide bond thus yielding PPMO product. This crude product was then purified by Strong Cation Exchange (SCX) catch and release chromatography, desalted, and lyophilized to a dry powder.

The activated coupling solutions were prepared by first weighing out the calculated amounts of CPP and coupling reagent. The CPP and coupling reagent were then combined using NMP and this solution was heated to 45° C. The DIPEA base was added to this solution and added to the appropriate oligonucleotide and allowed to react for 3 hours at room temperature.

Upon reacting for 3 hours the samples were diluted to 20 mL with Milli-Q water and purified by SCX chromatography (Source 30S Resin, GE HealthCare) on a BioRad Biologic LP MPLC system (Bio-Rad, Hercules, CA). A SCX gradient of X=30 or 50% the percentage of Buffer B is selected based on the number of positively charged residues in the peptide sequence to be conjugated. For the CPP of SEQ ID NO: 56, which contains five arginine residues, a gradient of 30% Buffer B was used. For the CPP of SEQ ID NO: 61, which contains eight arginine residues, a gradient of 50% Buffer B was used. The purification was run at a flow rate of 5 mL/min with fractions being collected every 0.53 minutes (2.65 mL per fraction). Using a 5 mL column, the 30 min gradient elution was over ˜30 Column Volumes (CV) with fractions being selected and pooled based on UV absorbance. The load effluent was collected and both the load effluent and product were desalted by SPE. Samples were eluted, then frozen with dry ice and lyophilized for 48 hours before being submitted for HPLC and mass spectrometry analysis.

TABLE 7
SCX purification gradient

| Time (min) | % A | % B |
|---|---|---|
| 0 | 100 | 0 |
| 1 | 100 | 0 |
| 30 | 100-X | X |
| 32 | 0 | 100 |
| 37 | 0 | 100 |
| 42 | 100 | 0 |

Buffer A: 20 mM NaH₂PO₄/25% ACN; pH 6.5
Buffer B: 1.5M Guanidine HCl/20 mM NaH₂PO₄/25% ACN; pH 6.5

### Example 4.—In vitro Assays

### 1. Exon-Skipping of PMP22 by 2′OMe AOs in Normal Fibroblasts

| Compound | | | SEQ | Exon- |
|---|---|---|---|---|
| No. | Sequence | Target Region | ID NO | skipping |
| 1 | CTGCGAGGAGAGCG | PMP22 H2A (−25−1) | 6 | + |
| | CTGGGCGTGAG | | | |
| 2 | AAGTTCTGCTCAGCG | PMP22 H2A (+1+25) | 7 | ++ |
| | GAGTTTCTGC | | | |
| 3 | CTCAGCAACAGGAG | PMP22 H2A (+30+54) | 9 | ++ |
| | GAGCATTCTGG | | | |
| 4 | GACGATGATACTCAG | PMP22 H2A (+40+64) | 13 | ++ |
| | CAACAGGAGG | | | |
| 5 | GAACAGCAGCACCA | PMP22 H2A (+70+94) | 27 | +++ |
| | GCACCGCGACG | | | |
| 6 | AGGCACTCACGCTGA | PMP22 H2D (+15−10) | 29 | + |
| | CGATCGTGGA | | | |
| 7 | CGATCCATTGCTAGA | PMP22 H3A (−15+10) | 30 | ++ |
| | GAGAATCAGA | | | |
| 8 | CGTGTCCATTGCCCA | PMP22 H3A (+1+25) | 31 | ++ |
| | CGATCCATTG | | | |
| 9 | ACAGTTCTGCCAGAG | PMP22 H3A (+24+48) | 33 | − |
| | ATCAGTTGCG | | | |
| 10 | GATGAGAAACAGTGG | PMP22 H3A (+65+89) | 35 | + |
| | TGGACATTTC | | | |
| 11 | CACCGTTTGGTGATG | PMP22 H3D (+22−3) | 38 | + |
| | ATGAGAAACA | | | |
| 12 | CAGACTGCAGCCATT | PMP22 H4A (−10+15) | 39 | − |
| | CTGGGGGAAA | | | |
| 13 | GAATGCTGAAGATGA | PMP22 H4A (+30+54) | 40 | + |
| | TCGACAGGAT | | | |
| 14 | AGAGTTGGCAGAAGA | PMP22 H4A (+60+84) | 41 | + |
| | ACAGGAACAG | | | |
| 15 | TGTAAAACCTGCCCC | PMP22 H4A | 42 | − |
| | CCTTGGTGAG | (+90+114) | | |

Normal fibroblasts cells were seeded in 24 well plates one day before transfection. On the day of transfection, 2′OMe antisense oligonucleotides (“AOs”) were complexed with Lipofectamine 3000 (L2K) (Life Technologies) in 50 μl of Opti-MEM (Life Technologies) according to the manufacturer's instruction (3 μl of Lipofectamine 3000/1 mL total transfection mix). The complexes were topped up to 1 mL with Opti-MEM and added to two wells (500 μl/well). Cells were collected after 24 hr incubation.

RT-PCR was performed on 50 ng of the RNA template using Superscript III One-Step RT-PCR System with Platinum® Taq DNA Polymerase (ThermoFisher Scientific, Australia). Cycling conditions include 55° C. for 30 minutes, 94° C. for 2 minutes followed by 28 cycles of 94° C. for 30 seconds, 55° C. for 30 seconds and 68° C. for 1 minute. Primers used were exon 1 forward (GGAAGAAGGGGTTACGCTGT (SEQ ID NO: 66)) and exon 5 reverse (GGAAGAAGGGGTTACGCTGT (SEQ ID NO: 67)).

Densitometry was performed and the exon skipping was calculated as a ratio of skipped transcriptions to total transcripts. −indicates <20% exon skipping, +indicates between 20-40% exon skipping, ++indicates between 40-80% exon skipping and +++indicates >80% exon skipping.

### 2. Exon-Skipping of PMP22 by a PPMOs Having CPP of SEQ ID NO: 61 in Normal Fibroblasts

| Compound | | | SEQ ID | Exon- |
|---|---|---|---|---|
| No. | Sequence | Target Region | NO | skipping |
| 16 | CACCGCGACGTGGA | PMP22 H2A (+55+79) | 23 | ++ |
| | GGACGATGATA | | | |
| 17 | CGATCCATTGCTAG | PMP22 H3A (−15+10) | 30 | ++ |
| | AGAGAATCAGA | | | |
| 18 | CGTGTCCATTGCCC | PMP22 H3A (+1+25) | 31 | − |
| | ACGATCCATTG | | | |
| 19 | AGAGTTGGCAGAAG | PMP22 H4A (+60+84) | 41 | ++ |
| | AACAGGAACAG | | | |

Normal fibroblast cells were seeded one day before in the growth media (10% FCS DMEM) and transfected with PPMO having CPP of SEQ ID NO: 61 diluted in Opti-MEM and left for 3-5 days before collecting cells.

RT-PCR was performed on 50 ng of the RNA template using Superscript III One-Step RT-PCR System with Platinum® Taq DNA Polymerase (ThermoFisher Scientific, Australia). Cycling conditions include 55° C. for 30 minutes, 94° C. for 2 minutes followed by 28 cycles of 94° C. for 30 seconds, 55° C. for 30 seconds and 68° C. for 1 minute. Primers used were exon 1 forward (GGAAGAAGGGGTTACGCTGT (SEQ ID NO: 66)) and exon 5 reverse (GGAAGAAGGGGTTACGCTGT (SEQ ID NO: 67)).

Densitometry was performed and the exon skipping was calculated as a ratio of skipped transcriptions to total transcripts. −indicates <20% exon skipping, +indicates between 20-40% exon skipping, ++indicates between 40-80% exon skipping and +++indicates >80% exon skipping.

### 3. Exon-Skipping of PMP22 by PPMOs Having CPP of SEQ ID NO: 56 in Normal Fibroblasts

| | | | SEQ | |
|---|---|---|---|---|
| Compound | | Target | ID | Exon- |
| No. | Sequence | Region | NO | skipping |
| 20 | CTCAGCAACAGGAGG | PMP22 H2A | 9 | ++ |
| | AGCATTCTGG | (+30 +54) | | |
| 21 | TGATACTCAGCAACA | PMP22 H2A | 10 | +++ |
| | GGAGGAGCAT | (+35 +59) | | |
| 22 | GACGATGATACTCAG | PMP22 H2A | 13 | ++ |
| | CAACAGGAGG | (+40 +64) | | |
| 23 | TGGAGGACGATGATA | PMP22 H2A | 16 | ++ |
| | CTCAGCAACA | (+45 +69) | | |
| 24 | CGACGTGGAGGACGA | PMP22 H2A | 20 | ++ |
| | TGATACTCAG | (+50 +74) | | |
| 25 | CACCGCGACGTGGAG | PMP22 H2A | 23 | ++ |
| | GACGATGATA | (+55 +79) | | |
| 26 | ACCAGCACCGCGACG | PMP22 H2A | 25 | +++ |
| | TGGAGGACGA | (+60 +84) | | |
| 27 | GCAGCACCAGCACCG | PMP22 H2A | 26 | ++ |
| | CGACGTGGAG | (+65 +89) | | |
| 28 | GAACAGCAGCACCAG | PMP22 H2A | 27 | ++ |
| | CACCGCGACG | (+70 +94) | | |
| 29 | GAGACGAACAGCAGC | PMP22 H2A | 28 | ++ |
| | ACCAGCACCG | (+75 +99) | | |

Normal fibroblast cells were seeded one day before in the growth media (10% FCS DMEM) and transfected with PPMOs having CPP of SEQ ID NO: 56 diluted in Opti-MEM and left for 3-5 days before collecting cells.

RT-PCR was performed on 50 ng of the RNA template using Superscript III One-Step RT-PCR System with Platinum® Taq DNA Polymerase (ThermoFisher Scientific, Australia). Cycling conditions include 55° C. for 30 minutes, 94° C. for 2 minutes followed by 28 cycles of 94° C. for 30 seconds, 55° C. for 30 seconds and 68° C. for 1 minute. Primers used were exon 1 forward (GGAAGAAGGGGTTACGCTGT (SEQ ID NO: 66)) and exon 5 reverse (GGAAGAAGGGGTTACGCTGT (SEQ ID NO: 67)).

Densitometry was performed and the exon skipping was calculated as a ratio of skipped transcriptions to total transcripts. −indicates <20% exon skipping, +indicates between 20-40% exon skipping, ++indicates between 40-80% exon skipping and +++indicates >80% exon skipping.

### 4. Exon-Skipping of PMP22 by PMOs in CMT1A Patient Fibroblasts

| | | | SEQ | |
|---|---|---|---|---|
| Compound | | Target | ID | Exon- |
| No. | Sequence | Region | NO | skipping |
| 30 | CAACAGGAGGAGCAT | PMP22 H2A | 8 | + |
| | TCTGGCGGCA | (+25 +49) | | |
| 31 | CTCAGCAACAGGAGG | PMP22 H2A | 9 | ++ |
| | AGCATTCTGG | (+30 +54) | | |
| 32 | TGATACTCAGCAACA | PMP22 H2A | 10 | ++ |
| | GGAGGAGCAT | (+35 +59) | | |
| 33 | CACCGCGACGTGGAG | PMP22 H2A | 23 | ++ |
| | GACGATGATA | (+55 +79) | | |
| 34 | CGATCCATTGCTAGA | PMP22 H3A | 30 | ++ |
| | GAGAATCAGA | (−15 +10) | | |
| 35 | CGTGTCCATTGCCCA | PMP22 H3A | 31 | ++ |
| | CGATCCATTG | (+1 +25) | | |
| 36 | AGAGTTGGCAGAAGA | PMP22 H4A | 41 | + |
| | ACAGGAACAG | (+60 +84) | | |

CMT1a fibroblast cells were resuspended in 20 μL of primary solution containing supplement and PMOs were delivered into the cells using Nucleofection/Neon electroporation and incubated in DMEM supplemented with 5% FCS for 24 hr before harvesting the cells for PMP22 transcript analysis using RT-PCR.

RT-PCR was performed on 50 ng of the RNA template using Superscript III One-Step RT-PCR System with Platinum® Taq DNA Polymerase (ThermoFisher Scientific, Australia). Cycling conditions include 55° C. for 30 minutes, 94° C. for 2 minutes followed by 28 cycles of 94° C. for 30 seconds, 55° C. for 30 seconds and 68° C. for 1 minute. Primers used were exon 1 forward (GGAAGAAGGGGTTACGCTGT (SEQ ID NO: 66)) and exon 5 reverse (GGAAGAAGGGGTTACGCTGT (SEQ ID NO: 67)).

Densitometry was performed and the exon skipping was calculated as a ratio of skipped transcriptions to total transcripts. −indicates <20% exon skipping, +indicates between 20-40% exon skipping, ++indicates between 40-80% exon skipping and ++indicates >80% exon skipping.

### 5. Exon-Skipping of PMP22 by PPMOs Having CPP of SEQ ID NO: 61 in CMT1A Patient Fibroblasts

| | | | SEQ | |
|---|---|---|---|---|
| Compound | | Target | ID | Exon- |
| No. | Sequence | Region | NO | skipping |
| 37 | CACCGCGACGTGGAG | PMP22 H2A | 23 | ++ |
| | GACGATGATA | (+55 +79) | | |
| 38 | CGATCCATTGCTAGA | PMP22 H3A | 30 | ++ |
| | GAGAATCAGA | (−15 +10) | | |
| 39 | CGTGTCCATTGCCCA | PMP22 H3A | 31 | − |
| | CGATCCATTG | (+1 +25) | | |
| 40 | AGAGTTGGCAGAAGA | PMP22 H4A | 41 | + |
| | ACAGGAACAG | (+60 +84) | | |

CMT1a fibroblast cells were seeded one day before in the growth media (10% FCS DMEM) and transfected with PPMOs having CPP of SEQ ID NO: 61 diluted in Opti-MEM and left for 3-5 days before collecting cells.

RT-PCR was performed on 50 ng of the RNA template using Superscript III One-Step RT-PCR System with Platinum® Taq DNA Polymerase (ThermoFisher Scientific, Australia). Cycling conditions include 55° C. for 30 minutes, 94° C. for 2 minutes followed by 28 cycles of 94° C. for 30 seconds, 55° C. for 30 seconds and 68° C. for 1 minute. Primers used were exon 1 forward (GGAAGAAGGGGTTACGCTGT (SEQ ID NO: 66)) and exon 5 reverse (GGAAGAAGGGGTTACGCTGT (SEQ ID NO: 67)).

Densitometry was performed and the exon skipping was calculated as a ratio of skipped transcriptions to total transcripts. −indicates <20% exon skipping, +indicates between 20-40% exon skipping, ++indicates between 40-80% exon skipping and +++indicates >80% exon skipping.

### 6. Exon-Skipping of PMP22 by PPMOs Having CPP of SEQ ID NO: 56 in CMT1A Patient Fibroblasts

| | | | SEQ | |
|---|---|---|---|---|
| Compound | | Target | ID | Exon- |
| No. | Sequence | Region | NO | skipping |
| 41 | CAACAGGAGGAGCAT | PMP22 H2A | 8 | ++ |
| | TCTGGCGGCA | (+25 +49) | | |
| 42 | CTCAGCAACAGGAGG | PMP22 H2A | 9 | ++ |
| | AGCATTCTGG | (+30 +54) | | |
| 43 | TGATACTCAGCAACA | PMP22 H2A | 10 | ++ |
| | GGAGGAGCAT | (+35 +59) | | |
| 44 | TGATACTCAGCAACA | PMP22 H2A | 12 | ++ |
| | GGAGG | (+40 +59) | | |
| 45 | GACGATGATACTCAG | PMP22 H2A | 13 | ++ |
| | CAACAGGAGG | (+40 +64) | | |
| 46 | GATGATACTCAGCAA | PMP22 H2A | 14 | ++ |
| | CAGGA | (+42 +61) | | |
| 47 | ACGATGATACTCAGC | PMP22 H2A | 15 | ++ |
| | AACAG | (+44 +63) | | |
| 48 | TGGAGGACGATGATA | PMP22 H2A | 16 | ++ |
| | CTCAGCAACA | (+45 +69) | | |
| 49 | GAGGACGATGATACT | PMP22 H2A | 18 | ++ |
| | CAGCA | (+48 +67) | | |
| 50 | TGGAGGACGATGATA | PMP22 H2A | 19 | ++ |
| | CTCAG | (+50 +69) | | |
| 51 | CGACGTGGAGGACGA | PMP22 H2A | 20 | ++ |
| | TGATACTCAG | (+50 +74) | | |
| 52 | CGTGGAGGACGATGA | PMP22 H2A | 21 | ++ |
| | TACTC | (+52 +71) | | |
| 53 | GACGTGGAGGACGAT | PMP22 H2A | 22 | ++ |
| | GATAC | (+54 +73) | | |
| 54 | CACCGCGACGTGGAG | PMP22 H2A | 23 | ++ |
| | GACGATGATA | (+55 +79) | | |
| 55 | GCGACGTGGAGGAC | PMP22 H2A | 24 | ++ |
| | GATGAT | (+56 +75) | | |
| 56 | ACCAGCACCGCGACG | PMP22 H2A | 25 | ++ |
| | TGGAGGACGA | (+60 +84) | | |
| 57 | GCAGCACCAGCACCG | PMP22 H2A | 26 | ++ |
| | CGACGTGGAG | (+65 +89) | | |
| 58 | GAACAGCAGCACCAG | PMP22 H2A | 27 | ++ |
| | CACCGCGACG | (+70 +94) | | |
| 59 | GAGACGAACAGCAGC | PMP22 H2A | 28 | ++ |
| | ACCAGCACCG | (+75 +99) | | |

CMT1a Normal fibroblast cells were seeded one day before in the growth media (10% FCS DMEM) and transfected with PPMOs having CPP of SEQ ID NO: 56 diluted in Opti-MEM and left for 3-5 days before collecting cells.

RT-PCR was performed on 50 ng of the RNA template using Superscript III One-Step RT-PCR System with Platinum® Taq DNA Polymerase (ThermoFisher Scientific, Australia). Cycling conditions include 55° C. for 30 minutes, 94° C. for 2 minutes followed by 28 cycles of 94° C. for 30 seconds, 55° C. for 30 seconds and 68° C. for 1 minute. Primers used were exon 1 forward (GGAAGAAGGGGTTACGCTGT (SEQ ID NO: 66)) and exon 5 reverse (GGAAGAAGGGGTTACGCTGT (SEQ ID NO: 67)).

Densitometry was performed and the exon skipping was calculated as a ratio of skipped transcriptions to total transcripts. −indicates <20% exon skipping, +indicates between 20-40% exon skipping, ++indicates between 40-80% exon skipping and indicates >80% exon skipping.

### 7. Exon-Skipping of PMP22 by PPMOs Having CPP of SEQ ID NO: 61 in Human Schwann Cell Line

| | | | SEQ | |
|---|---|---|---|---|
| Compound | | Target | ID | Exon- |
| No. | Sequence | Region | NO | skipping |
| 60 | CACCGCGACGTGGAGG | PMP22 H2A | 23 | ++ |
| | ACGATGATA | (+55 +79) | | |
| 61 | CGATCCATTGCTAGAG | PMP22 H3A | 30 | ++ |
| | AGAATCAGA | (−15 +10) | | |
| 62 | CGTGTCCATTGCCCAC | PMP22 H3A | 31 | + |
| | GATCCATTG | (+1 +25) | | |
| 63 | AGAGTTGGCAGAAGAA | PMP22 H4A | 41 | + |
| | CAGGAACAG | (+60 +84) | | |

Normal Schwann cells were seeded one day before in the growth media (10% FCS DMEM) and transfected with PPMOs having CPP of SEQ ID NO: 61 diluted in Opti-MEM and left for 3-5 days before collecting cells.

RT-PCR was performed on 50 ng of the RNA template using Superscript III One-Step RT-PCR System with Platinum® Taq DNA Polymerase (ThermoFisher Scientific, Australia). Cycling conditions include 55° C. for 30 minutes, 94° C. for 2 minutes followed by 28 cycles of 94° C. for 30 seconds, 55° C. for 30 seconds and 68° C. for 1 minute. Primers used were exon 1 forward (GGAAGAAGGGGTTACGCTGT (SEQ ID NO: 66)) and exon 5 reverse (GGAAGAAGGGGTTACGCTGT (SEQ ID NO: 67)).

Densitometry was performed and the exon skipping was calculated as a ratio of skipped transcriptions to total transcripts. −indicates <20% exon skipping, +indicates between 20-40% exon skipping, ++indicates between 40-80% exon skipping and +++indicates >80% exon skipping.

### 8. Exon-Skipping of PMP22 by PPMOs Having CPP of SEQ ID NO: 56 in Human Schwann Cell Line

| | | | SEQ | |
|---|---|---|---|---|
| Compound | | Target | ID | Exon- |
| No. | Sequence | Region | NO | skipping |
| 64 | CAACAGGAGGAGCAT | PMP22 H2A | 8 | ++ |
| | TCTGGCGGCA | (+25 +49) | | |
| 65 | CTCAGCAACAGGAGG | PMP22 H2A | 9 | ++ |
| | AGCATTCTGG | (+30 +54) | | |
| 66 | TGATACTCAGCAACAG | PMP22 H2A | 10 | ++ |
| | GAGGAGCAT | (+35 +59) | | |
| 67 | TGATACTCAGCAACAG | PMP22 H2A | 12 | ++ |
| | GAGG | (+40 +59) | | |
| 68 | GACGATGATACTCAGC | PMP22 H2A | 13 | ++ |
| | AACAGGAGG | (+40 +64) | | |
| 69 | GATGATACTCAGCAAC | PMP22 H2A | 14 | ++ |
| | AGGA | (+42 +61) | | |
| 70 | ACGATGATACTCAGCA | PMP22 H2A | 15 | ++ |
| | ACAG | (+44 +63) | | |
| 71 | TGGAGGACGATGATA | PMP22 H2A | 16 | ++ |
| | CTCAGCAACA | (+45 +69) | | |
| 72 | GAGGACGATGATACTC | PMP22 H2A | 18 | ++ |
| | AGCA | (+48 +67) | | |
| 73 | TGGAGGACGATGATA | PMP22 H2A | 19 | + |
| | CTCAG | (+50 +69) | | |
| 74 | CGACGTGGAGGACGA | PMP22 H2A | 20 | ++ |
| | TGATACTCAG | (+50 +74) | | |
| 75 | CGTGGAGGACGATGA | PMP22 H2A | 21 | ++ |
| | TACTC | (+52 +71) | | |
| 76 | GACGTGGAGGACGAT | PMP22 H2A | 22 | ++ |
| | GATAC | (+54 +73) | | |
| 77 | CACCGCGACGTGGAG | PMP22 H2A | 23 | ++ |
| | GACGATGATA | (+55 +79) | | |
| 78 | GCGACGTGGAGGACG | PMP22 H2A | 24 | ++ |
| | ATGAT | (+56 +75) | | |
| 79 | ACCAGCACCGCGACG | PMP22 H2A | 25 | ++ |
| | TGGAGGACGA | (+60 +84) | | |
| 80 | GCAGCACCAGCACCG | PMP22 H2A | 26 | ++ |
| | CGACGTGGAG | (+65 +89) | | |
| 81 | GAACAGCAGCACCAG | PMP22 H2A | 27 | ++ |
| | CACCGCGACG | (+70 +94) | | |
| 82 | GAGACGAACAGCAGC | PMP22 H2A | 28 | ++ |
| | ACCAGCACCG | (+75 +99) | | |

Normal Schwann cells were seeded one day before in the growth media (10% FCS DMEM) and transfected with PPMOs having CPP of SEQ ID NO: 56 diluted in Opti-MEM and left for 3-5 days before collecting cells.

RT-PCR was performed on 50 ng of the RNA template using Superscript III One-Step RT-PCR System with Platinum® Taq DNA Polymerase (ThermoFisher Scientific, Australia). Cycling conditions include 55° C. for 30 minutes, 94° C. for 2 minutes followed by 28 cycles of 94° C. for 30 seconds, 55° C. for 30 seconds and 68° C. for 1 minute. Primers used were exon 1 forward (GGAAGAAGGGGTTACGCTGT (SEQ ID NO: 66)) and exon 5 reverse (GGAAGAAGGGGTTACGCTGT (SEQ ID NO: 67)).

Densitometry was performed and the exon skipping was calculated as a ratio of skipped transcriptions to total transcripts. −indicates <20% exon skipping, +indicates between 20-40% exon skipping, ++indicates between 40-80% exon skipping and +++indicates >80% exon skipping.

### 9. Protein Reduction of PMP22 by PPMOs Having CPP of SEQ ID NO: 56 in Normal Fibroblasts

| | | | SEQ | |
|---|---|---|---|---|
| Compound | | Target | ID | Exon- |
| No. | Sequence | Region | NO | skipping |
| 83 | CTCAGCAACAGGAGGA | PMP22 H2A | 9 | − |
| | GCATTCTGG | (+30 +54) | | |
| 84 | TGATACTCAGCAACAG | PMP22 H2A | 10 | − |
| | GAGGAGCAT | (+35 +59) | | |
| 85 | GCAGCACCAGCACCGC | PMP22 H2A | 26 | +++ |
| | GACGTGGAG | (+65 +89) | | |
| 86 | GAACAGCAGCACCAGC | PMP22 H2A | 27 | − |
| | ACCGCGACG | (+70 +94) | | |
| 87 | GAGACGAACAGCAGCA | PMP22 H2A | 28 | − |
| | CCAGCACCG | (+75 +99) | | |

Normal fibroblast cells were seeded one day before in the growth media (10% FCS DMEM) and transfected with PPMOs having CPP of SEQ ID NO: 56 diluted in Opti-MEM and left for 3-5 days before collecting cells.

Approximately 30 μg total protein was used for each sample and transferred onto PDVF membrane using iBlot™ 2 Gel Transfer Device (Thermo Fisher). PMP22 was detected with polyclonal anti-PMP22 (Origene) applied at a dilution of 1:500 for 48 hours at 4° C. β tubulin was detected with monoclonal anti β Tubulin (Thermo Fisher) at a dilution of 1:3000 for 48 hours at 4° C. HRP-labelled anti-rabbit and anti-mouse secondary antibodies were applied respectively for 1 hour at room temperature. The blots were detected using Immobilon western chemiluminescent HRP substrate and images were captured using a Fusion FX gel documentation system (Vilber Lourmat) with FusionCapt Advance software. Image J software (NIH) was used for densitometric analysis.

Densitometry was performed and relative protein quantities was calculated by normalizing to β tubulin. The relative change from the untransfected samples is used to show the protein reduction of PMP22. −indicates no protein reduction, +indicates between <20% protein reduction, ++indicates between 20-50% protein reduction and +++indicates >50% protein reduction.

### 10. Protein Reduction of PMP22 by PPMOs Having CPP of SEQ ID NO: 56 in CMT1A Patient Fibroblasts

| | | | SEQ | |
|---|---|---|---|---|
| Compound | | Target | ID | Exon- |
| No. | Sequence | Region | NO | skipping |
| 88 | CGACGTGGAGGACGA | PMP22 H2A | 20 | − |
| | TGATACTCAG | (+50 +74) | | |
| 89 | CACCGCGACGTGGAG | PMP22 H2A | 23 | − |
| | GACGATGATA | (+55 +79) | | |

CMT1A fibroblast cells were seeded one day before in the growth media (10% FCS DMEM) and transfected with PPMOs having CPP of SEQ ID NO: 56 diluted in Opti-MEM and left for 3-5 days before collecting cells.

Approximately 30 μg total protein was used for each sample and transferred onto PDVF membrane using iBlot™ 2 Gel Transfer Device (Thermo Fisher). PMP22 was detected with polyclonal anti-PMP22 (Origene) applied at a dilution of 1:500 for 48 hours at 4° C. B tubulin was detected with monoclonal anti β Tubulin (Thermo Fisher) at a dilution of 1:3000 for 48 hours at 4° C. HRP-labelled anti-rabbit and anti-mouse secondary antibodies were applied respectively for 1 hour at room temperature. The blots were detected using Immobilon western chemiluminescent HRP substrate and images were captured using a Fusion FX gel documentation system (Vilber Lourmat) with FusionCapt Advance software. Image J software (NIH) was used for densitometric analysis.

Densitometry was performed and relative protein quantities was calculated by normalizing to β tubulin. The relative change from the untransfected samples is used to show the protein reduction of PMP22. −indicates no protein reduction, +indicates between <20% protein reduction, ++indicates between 20-50% protein reduction and +++indicates >50% protein reduction.

### 11. Protein Reduction of PMP22 by PPMOs Having CPP of SEQ ID NO: 61 in CMT1A Patient Fibroblasts

| | | | SEQ | |
|---|---|---|---|---|
| Compound | | Target | ID | Exon- |
| No. | Sequence | Region | NO | skipping |
| 90 | CTCAGCAACAGGAGG | PMP22 H2A | 9 | +++ |
| | AGCATTCTGG | (+55 +79) | | |
| 91 | CGATCCATTGCTAGAG | PMP22 H3A | 30 | + |
| | AGAATCAGA | (−15 +10) | | |
| 92 | AGAGTTGGCAGAAGAA | PMP22 H4A | 41 | ++ |
| | CAGGAACAG | (+60 +84) | | |

CMT1A fibroblast cells were seeded one day before in the growth media (10% FCS DMEM) and transfected with PPMOs having CPP of SEQ ID NO: 61 diluted in Opti-MEM and left for 3-5 days before collecting cells.

Approximately 30 μg total protein was used for each sample and transferred onto PDVF membrane using iBlot™ 2 Gel Transfer Device (Thermo Fisher). PMP22 was detected with polyclonal anti-PMP22 (Origene) applied at a dilution of 1:500 for 48 hours at 4° C. β tubulin was detected with monoclonal anti β Tubulin (Thermo Fisher) at a dilution of 1:3000 for 48 hours at 4° C. HRP-labelled anti-rabbit and anti-mouse secondary antibodies were applied respectively for 1 hour at room temperature. The blots were detected using Immobilon western chemiluminescent HRP substrate and images were captured using a Fusion FX gel documentation system (Vilber Lourmat) with FusionCapt Advance software. Image J software (NIH) was used for densitometric analysis.

Densitometry was performed and relative protein quantities was calculated by normalizing to β tubulin. The relative change from the untransfected samples is used to show the protein reduction of PMP22. −indicates no protein reduction, +indicates between <20% protein reduction, ++indicates between 20-50% protein reduction and +++indicates >50% protein reduction.

### 12. Protein Reduction of PMP22 by PPMOs Having CPP of SEQ ID NO: 61 in Human Schwann Cell Line

| | | | SEQ | |
|---|---|---|---|---|
| Compound | | Target | ID | Exon- |
| No. | Sequence | Region | NO | skipping |
| 93 | CTCAGCAACAGGAGG | PMP22 H2A | 9 | + |
| | AGCATTCTGG | (+55 +79) | | |
| 94 | CGATCCATTGCTAGAG | PMP22 H3A | 30 | ++ |
| | AGAATCAGA | (−15 +10) | | |
| 95 | AGAGTTGGCAGAAGA | PMP22 H4A | 41 | ++ |
| | ACAGGAACAG | (+60 +84) | | |

Human Schwann Cells were seeded one day before in the growth media (10% FCS DMEM) and transfected with PPMOs having CPP of SEQ ID NO: 61 diluted in Opti-MEM and left for 3-5 days before collecting cells.

Approximately 30 μg total protein was used for each sample and transferred onto PDVF membrane using iBlot™ 2 Gel Transfer Device (Thermo Fisher). PMP22 was detected with polyclonal anti-PMP22 (Origene) applied at a dilution of 1:500 for 48 hours at 4° C. B tubulin was detected with monoclonal anti β Tubulin (Thermo Fisher) at a dilution of 1:3000 for 48 hours at 4° C. HRP-labelled anti-rabbit and anti-mouse secondary antibodies were applied respectively for 1 hour at room temperature. The blots were detected using Immobilon western chemiluminescent HRP substrate and images were captured using a Fusion FX gel documentation system (Vilber Lourmat) with FusionCapt Advance software. Image J software (NIH) was used for densitometric analysis.

Densitometry was performed and relative protein quantities was calculated by normalizing to β tubulin. The relative change from the untransfected samples is used to show the protein reduction of PMP22. −indicates no protein reduction, +indicates between <20% protein reduction, ++indicates between 20-50% protein reduction and +++indicates >50% protein reduction.

13. Exon-Skipping of PMP22 by PPMOs Having CPP of SEQ ID NO: 61 in Schwann Cells Isolated from CMT1A C3 Mice

| | | SEQ | |
|---|---|---|---|
| | Target | ID | Exon- |
| Sequence | Region | NO | skipping |
| GACGATGATACTCAGCAACAGGAGG | PMP22 H2A | 13 | − |
| | (+40 +64) | | |
| TGGAGGACGATGATACTCAGCAACA | PMP22 H2A | 16 | ++ |
| | (+45 +69) | | |
| CGACGTGGAGGACGATGATACTCAG | PMP22 H2A | 20 | +++ |
| | (+50 +74) | | |
| CACCGCGACGTGGAGGACGATGATA | PMP22 H2A | 23 | +++ |
| | (+55 +79) | | |
| CGATCCATTGCTAGAGAGAATCAGA | PMP22 H3A | 30 | +++ |
| | (−15 +10) | | |
| AGAGTTGGCAGAAGAACAGGAACAG | PMP22 H4A | 41 | ++ |
| | (+60 +84) | | |

Schwann cells were isolated from CMT1A C₃ mice and were seeded one day before in the growth media (10% FCS DMEM) and transfected with PPMOs having CPP of SEQ ID NO: 61 diluted in Opti-MEM and left for 3-5 days before collecting cells.

RT-PCR was performed on 50 ng of the RNA template using Superscript III One-Step RT-PCR System with Platinum® Taq DNA Polymerase (ThermoFisher Scientific, Australia). Cycling conditions include 55° C. for 30 minutes, 94° C. for 2 minutes followed by 28 cycles of 94° C. for 30 seconds, 55° C. for 30 seconds and 68° C. for 1 minute. Primers used were exon 1 forward (GGAAGAAGGGGTTACGCTGT) and exon 5 reverse (GGAAGAAGGGGTTACGCTGT).

Densitometry was performed and the exon skipping were calculated as a ratio of skipped transcriptions to total transcripts. −indicates <20% exon skipping, +indicates between 20-40% exon skipping, ++indicates between 40-80% exon skipping and +++indicates >80% exon skipping.

14. Exon-Skipping of PMP22 by PPMOs Having CPP of SEQ ID NO: 58 in Schwann Cells Isolated from CMT1A C3 Mice

| | | SEQ | |
|---|---|---|---|
| | Target | ID | Exon- |
| Sequence | Region | NO | skipping |
| GACGATGATACTCAGCAACAGGAGG | PMP22 H2A | 13 | +++ |
| | (+40 +64) | | |
| TGGAGGACGATGATACTCAGCAACA | PMP22 H2A | 16 | +++ |
| | (+45 +69) | | |
| CGATCCATTGCTAGAGAGAATCAGA | PMP22 H3A | 30 | ++ |
| | (−15 +10) | | |
| AGAGTTGGCAGAAGAACAGGAACAG | PMP22 H4A | 41 | ++ |
| | (+60 +84) | | |

Schwann cells were isolated from CMT1A C3 mice and were seeded one day before in the growth media (10% FCS DMEM) and transfected with PPMOs having CPP of SEQ ID NO: 58 diluted in Opti-MEM and left for 3-5 days before collecting cells.

RT-PCR was performed on 50 ng of the RNA template using Superscript III One-Step RT-PCR System with Platinum® Taq DNA Polymerase (ThermoFisher Scientific, Australia). Cycling conditions include 55° C. for 30 minutes, 94° C. for 2 minutes followed by 28 cycles of 94° C. for 30 seconds, 55° C. for 30 seconds and 68° C. for 1 minute. Primers used were exon 1 forward (GGAAGAAGGGGTTACGCTGT) and exon 5 reverse (GGAAGAAGGGGTTACGCTGT).

Densitometry was performed and the exon skipping were calculated as a ratio of skipped transcriptions to total transcripts. −indicates <20% exon skipping, +indicates between 20-40% exon skipping, ++indicates between 40-80% exon skipping and +++indicates >80% exon skipping.

15. Protein Reduction of PMP22 by PPMOs Having CPP of SEQ ID NO: 61 in Schwann Cells Isolated from CMT1A C3 Mice

| | | SEQ | |
|---|---|---|---|
| | Target | ID | Protein |
| Sequence | Region | NO | reduction |
| TGGAGGACGATGATACTCAGCAACA | PMP22 H2A | 16 | + |
| | (+45 +69) | | |

Schwann cells were isolated from CMT1A C3 mice and were seeded one day before in the growth media (10% FCS DMEM) and transfected with PPMOs having CPP of SEQ ID NO: 61 diluted in Opti-MEM and left for 3-5 days before collecting cells.

Approximately 30 μg total protein was used for each sample and transferred onto PDVF membrane using iBlot™ 2 Gel Transfer Device (Thermo Fisher). PMP22 was detected with polyclonal anti-PMP22 (Origene) applied at a dilution of 1:500 for 48 hours at 4° C. β tubulin was detected with monoclonal anti β Tubulin (Thermo Fisher) at a dilution of 1:3000 for 48 hours at 4° C. HRP-labelled anti-rabbit and anti-mouse secondary antibodies were applied respectively for 1 hour at room temperature. The blots were detected using Immobilon western chemiluminescent HRP substrate and images were captured using a Fusion FX gel documentation system (Vilber Lourmat) with FusionCapt Advance software. Image J software (NIH) was used for densitometric analysis.

Densitometry was performed and relative protein quantities were calculated by normalizing to β tubulin. The relative change from the untransfected samples is used to show the protein reduction of PMP22. −indicates no protein reduction, +indicates between <20% protein reduction, ++indicates between 20-50% protein reduction and +++indicates >50% protein reduction.

16. Protein Reduction of PMP22 by PPMOs Having CPP of SEQ ID NO: 58 in Schwann Cells Isolated from CMT1A C3 Mice

| | | SEQ | |
|---|---|---|---|
| | Target | ID | Protein |
| Sequence | Region | NO | reduction |
| TGGAGGACGATGATACTCAGCAACA | PMP22 H2A | 16 | + |
| | (+45 +69) | | |
| CGATCCATTGCTAGAGAGAATCAGA | PMP22 H3A | 30 | + |
| | (−15 +10) | | |

Schwann cells were isolated from CMT1A C3 mice and were seeded one day before in the growth media (10% FCS DMEM) and transfected with PPMOs having CPP of SEQ ID NO: 58 diluted in Opti-MEM and left for 3-5 days before collecting cells.

Approximately 30 μg total protein was used for each sample and transferred onto PDVF membrane using iBlot™ 2 Gel Transfer Device (Thermo Fisher). PMP22 was detected with polyclonal anti-PMP22 (Origene) applied at a dilution of 1:500 for 48 hours at 4° C. β tubulin was detected with monoclonal anti β Tubulin (Thermo Fisher) at a dilution of 1:3000 for 48 hours at 4° C. HRP-labelled anti-rabbit and anti-mouse secondary antibodies were applied respectively for 1 hour at room temperature. The blots were detected using Immobilon western chemiluminescent HRP substrate and images were captured using a Fusion FX gel documentation system (Vilber Lourmat) with FusionCapt Advance software. Image J software (NIH) was used for densitometric analysis.

Densitometry was performed and relative protein quantities were calculated by normalizing to β tubulin. The relative change from the untransfected samples is used to show the protein reduction of PMP22. −indicates no protein reduction, +indicates between <20% protein reduction, ++indicates between 20-50% protein reduction and +++indicates >50% protein reduction.

1. An antisense oligomer comprising a chemically modified antisense oligomer having a targeting sequence that is complementary to a target region of the human peripheral myelin protein 22 (PMP22) pre-mRNA.

2. The antisense oligomer of claim 1, wherein the antisense oligomer induces skipping of one or more of exon 2 (SEQ ID NO: 2), exon 3 (SEQ ID NO: 3), exon 4 (SEQ ID NO: 4), or exon 5 (SEQ ID NO: 5) of the PMP22 pre-mRNA.

3. The antisense oligomer of claim 1, wherein the targeting sequence is complementary to a region within one of exon 2 (SEQ ID NO: 2), exon 3 (SEQ ID NO: 3), exon 4 (SEQ ID NO: 4), or exon 5 (SEQ ID NO: 5).

4. The antisense oligomer of claim 1, wherein the targeting sequence is complementary to a region spanning an exon/intron junction of exon 2 (SEQ ID NO: 2), exon 3 (SEQ ID NO: 3), exon 4 (SEQ ID NO: 4), or exon 5 (SEQ ID NO: 5).

5. The antisense oligomer of any one of claims 1-4, wherein the target region is PMP22 H2A (−25−1), PMP22 H2A (+1+25), PMP22 H2A (+25+49), PMP22 H2A (+30+54), PMP22 H2A (+35+59), PMP22 H2A (+38+57), PMP22 H2A (+40+59), PMP22 H2A (+40+64), PMP22 H2A (+42+61), PMP22 H2A (+44+63), PMP22 H2A (+45+69), PMP22 H2A (+46+65), PMP22 H2A (+48+67), PMP22 H2A (+50+69), PMP22 H2A (+50+74), PMP22 H2A (+52+71), PMP22 H2A (+54+73), PMP22 H2A (+55+79), PMP22 H2A (+56+75), PMP22 H2A (+60+84), PMP22 H2A (+65+89), PMP22 H2A (+70+94), PMP22 H2A (+75+99), PMP22 H2D (+15−10), PMP22 H3A (−15+10), PMP22 H3A (+1+25), PMP22 H3A (+15+39), PMP22 H3A (+24+48), PMP22 H3A (+48+72), PMP22 H3A (+65+89), PMP22 H3A (+74+98), PMP22 H3D (+17−8), PMP22 H3D (+22−3), PMP22 H4A (−10+15), PMP22 H4A (+30+54), PMP22 H4A (+60+84), PMP22 H4A (+90+114), PMP22 H4A (+100+124), PMP22 H4A (+110+134), PMP22 H4D (+22−3), PMP22 H5A (−8+17), PMP22 H5A (+18+42), PMP22 H5A (+37+61), PMP22 H5A (+55+79), or PMP22 H5A (+1271+1295).

6. The antisense oligomer of any one of claims 1-5, wherein the targeting sequence is selected from SEQ ID NOs: 6 to 50.

7. The antisense oligomer of any one of claims 1-6, wherein the antisense oligomer is complementary to a portion of, or induces skipping of, exon 2.

8. The antisense oligomer of claim 7, wherein the target region is PMP22 H2A (−25−1), PMP22 H2A (+1+25), PMP22 H2A (+25+49), PMP22 H2A (+30+54), PMP22 H2A (+35+59), PMP22 H2A (+40+64), PMP22 H2A (+45+69), PMP22 H2A (+50+74), PMP22 H2A (+55+79), PMP22 H2A (+60+84), PMP22 H2A (+65+89), PMP22 H2A (+70+94), PMP22 H2A (+75+99), or PMP22 H2D (+15−10).

9. The antisense oligomer of claim 8, wherein the antisense oligomer comprises a targeting sequence selected from SEQ ID NOs: 6 to 29.

10. The antisense oligomer of any one of claims 1-6, wherein the antisense oligomer is complementary to a portion of, or induces skipping of, exon 3.

11. The antisense oligomer of claim 10, wherein the target region is PMP22 H3A (−15+10), PMP22 H3A (+1+25), PMP22 H3A (+15+39), PMP22 H3A (+24+48), PMP22 H3A (+48+72), PMP22 H3A (+65+89), PMP22 H3A (+74+98), PMP22 H3D (+17−8), or PMP22 H3D (+22−3).

12. The antisense oligomer of claim 11, wherein the antisense oligomer comprises a targeting sequence selected from SEQ ID NOs: 30 to 38.

13. The antisense oligomer of any one of claims 1-6, wherein the antisense oligomer is complementary to a portion of, or induces skipping of, exon 4.

14. The antisense oligomer of claim 13, wherein the target region is PMP22 H4A (−10+15), PMP22 H4A (+30+54), PMP22 H4A (+60+84), PMP22 H4A (+90+114), PMP22 H4A (+100+124), PMP22 H4A (+110+134), or PMP22 H4D (+22−3).

15. The antisense oligomer of claim 14, wherein the antisense oligomer comprises a targeting sequence selected from SEQ ID NOs: 39 to 45.

16. The antisense oligomer of any one of claims 1-6, wherein the antisense oligomer is complementary to a portion of, or induces skipping of, exon 5.

17. The antisense oligomer of claim 16, wherein the target region is PMP22 H5A (−8+17), PMP22 H5A (+18+42), PMP22 H5A (+37+61), PMP22 H5A (+55+79), or PMP22 H5A (+1271+1295).

18. The antisense oligomer of claim 17, wherein the antisense oligomer comprises a targeting sequence selected from SEQ ID NOs: 46 to 50.

19. The antisense oligomer of any one of claims 1-18, wherein the antisense oligomer is covalently linked to a cell-penetrating peptide.

20. The antisense oligomer of claim 19, wherein the cell-penetrating peptide is covalently linked to the antisense oligomer via a linker selected from a direct bond, a glycine, or a proline.

21. The antisense oligomer of claim 19 or claim 20, wherein the cell-penetrating peptide is selected from rTAT, Tat, R₉F₂, R₅F₂R₄, R₄, R₅, R₆, R₇, R₈, R₉, (RXR)₄, (RXR)₅, (RXRRBR)₂, (RAR)₄F₂, and (RGR)₄F₂, wherein A represents alanine, B represents beta alanine, F represents phenylalanine, G represents glycine, R represents arginine, and X represents 6-aminohexanoic acid

22. The antisense oligomer of any one of claims 1-21, wherein the antisense oligomer is selected from a peptide nucleic acid, a locked nucleic acid, phosphorodiamidate morpholino oligomer, a 2′-O-Me phosphorothioate oligomer, or a combination thereof.

23. The antisense oligomer of claim 22, wherein the antisense oligomer is a phosphorodiamidate morpholino oligomer.

24. An antisense oligomer having a targeting sequence that is complementary to a portion of one or more of exon 2 (SEQ ID NO: 2), exon 3 (SEQ ID NO: 3), exon 4 (SEQ ID NO: 4), or exon 5 (SEQ ID NO: 5) of the human peripheral myelin protein 22 pre-mRNA, wherein the antisense oligomer is a phosphorodiamidate morpholino oligonucleotide of Formula I: 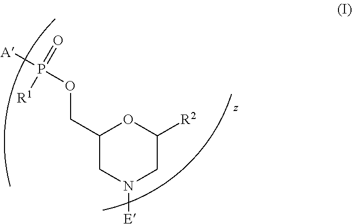
or a pharmaceutically acceptable salt thereof,
wherein:
A′ is selected from —NHCH₂C(O)NH₂, —N(C₁₋₆-alkyl)CH₂C(O)NH₂, 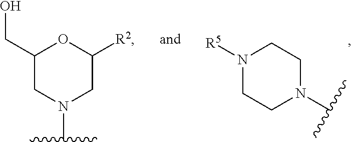
wherein
R₅ is —C(O)(O-alkyl)ₓ-OH, wherein x is 3-10, and each alkyl group is independently at each occurrence C₂₋₆-alkyl, or R⁵ is selected from —C(O)C₁₋₆ alkyl, trityl, monomethoxytrityl, —(C₁₋₆-alkyl)R⁶, —(C₁₋₆ heteroalkyl)-R⁶, aryl-R⁶, heteroaryl-R⁶, —C(O)O—(C₁₋₆ alkyl)-R⁶, —C(O)O-aryl-R⁶, —C(O)O-heteroaryl-R⁶, and 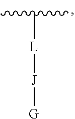
wherein R⁶ is selected from OH, SH, and NH₂, or R⁶ is O, S, or NH, covalently linked to a solid support;
each R¹ is independently selected from OH and —NR³R⁴, wherein each R³ and R⁴ is independently at each occurrence H, —C₁₋₆ alkyl, or wherein R³ and R⁴ taken together represent an optionally substituted piperazine, piperidine, or pyrrolidine, wherein the piperazine has the formula of: 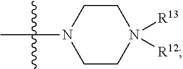
R¹² is H, C₁-C₆ alkyl, or an electron pair;
R¹³ is selected from the group consisting of H, C₁-C₆ alkyl, C(═NH)NH₂, Z-L²-NHC(═NH)NH₂, and [C(O)CHR′NH]ₘH;
Z is a carbonyl or direct bond;
L² is an optional linker selected from C₁-C₁₈ alkyl, C₁-C₁₈ alkoxy, and C₁-C₁₈ alkylamino;
R′ is a side chain of a naturally occurring amino acid or a one- or two-carbon homolog thereof;
m is 1-6;
each R² is independently selected from a naturally or non-naturally occurring nucleobase and the sequence formed by the combination of each R² from 5′ to 3′ is a targeting sequence;
z is 8-40;
E′ is selected from H, —C₁₋₆ alkyl, —C(O)C₁₋₆ alkyl, benzoyl, stearoyl, trityl, monomethoxytrityl, dimethoxytrityl, trimethoxytrityl, 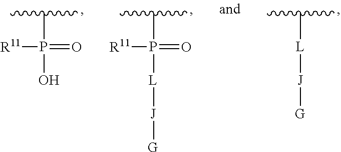
wherein
R¹¹ is selected from OH and —NR³R⁴,
wherein L is covalently linked by an amide bond to the carboxy-terminus of J, and L is selected from —NH(CH₂)₁₋₆C(O)—, —NH(CH₂)₁₋₆C(O)NH(CH₂)₁₋₆C(O)—, and 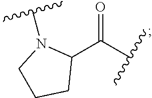 [SMILES: *C(=O)C1CCCN1*]
J is a carrier peptide;
G is selected from H, —C(O)C₁₋₆ alkyl, benzoyl, and stearoyl, and G is covalently linked to the amino-terminus of J.

25. The antisense oligomer of claim 24, wherein the antisense oligomer or induces skipping of one or more of exon 2 (SEQ ID NO: 2), exon 3 (SEQ ID NO: 3), exon 4 (SEQ ID NO: 4), or exon 5 (SEQ ID NO: 5) of the PMP22 pre-mRNA.

26. The antisense oligomer of claim 24, wherein the targeting sequence is complementary to a region within one of exon 2 (SEQ ID NO: 2), exon 3 (SEQ ID NO: 3), exon 4 (SEQ ID NO: 4), or exon 5 (SEQ ID NO: 5).

27. The antisense oligomer of claim 24, wherein the targeting sequence is complementary to a region spanning an exon/intron junction of exon 2 (SEQ ID NO: 2), exon 3 (SEQ ID NO: 3), exon 4 (SEQ ID NO: 4), or exon 5 (SEQ ID NO: 5).

28. The antisense oligomer of any one of claims 24-27, wherein the target region is PMP22 H2A (−25−1), PMP22 H2A (+1+25), PMP22 H2A (+25+49), PMP22 H2A (+30+54), PMP22 H2A (+35+59), PMP22 H2A (+38+57), PMP22 H2A (+40+59), PMP22 H2A (+40+64), PMP22 H2A (+42+61), PMP22 H2A (+44+63), PMP22 H2A (+45+69), PMP22 H2A (+46+65), PMP22 H2A (+48+67), PMP22 H2A (+50+69), PMP22 H2A (+50+74), PMP22 H2A (+52+71), PMP22 H2A (+54+73), PMP22 H2A (+55+79), PMP22 H2A (+56+75), PMP22 H2A (+60+84), PMP22 H2A (+65+89), PMP22 H2A (+70+94), PMP22 H2A (+75+99), PMP22 H2D (+15−10), PMP22 H3A (−15+10), PMP22 H3A (+1+25), PMP22 H3A (+15+39), PMP22 H3A (+24+48), PMP22 H3A (+48+72), PMP22 H3A (+65+89), PMP22 H3A (+74+98), PMP22 H3D (+17−8), PMP22 H3D (+22−3), PMP22 H4A (−10+15), PMP22 H4A (+30+54), PMP22 H4A (+60+84), PMP22 H4A (+90+114), PMP22 H4A (+100+124), PMP22 H4A (+110+134), PMP22 H4D (+22−3), PMP22 H5A (−8+17), PMP22 H5A (+18+42), PMP22 H5A (+37+61), PMP22 H5A (+55+79), or PMP22 H5A (+1271+1295).

29. The antisense oligomer of any one of claims 24-28, wherein the targeting sequence is selected from:
(SEQ ID NO: 6)
CTGCGAGGAGAGCGCTGGGCGTGAG,
z is 25;
(SEQ ID NO: 7)
AAGTTCTGCTCAGCGGAGTTTCTGC,
z is 25;
(SEQ ID NO: 8)
CAACAGGAGGAGCATTCTGGCGGCA,
z is 25;
(SEQ ID NO: 9)
CTCAGCAACAGGAGGAGCATTCTGG,
z is 25;
(SEQ ID NO: 10)
TGATACTCAGCAACAGGAGGAGCAT,
z is 25;
(SEQ ID NO: 11)
ATACTCAGCAACAGGAGGAG,
z is 20;
(SEQ ID NO: 12)
TGATACTCAGCAACAGGAGG,
z is 20;
(SEQ ID NO: 13)
GACGATGATACTCAGCAACAGGAGG,
z is 25;
(SEQ ID NO: 14)
GATGATACTCAGCAACAGGA,
z is 20;
(SEQ ID NO: 15)
ACGATGATACTCAGCAACAG,
z is 20;
(SEQ ID NO: 16)
TGGAGGACGATGATACTCAGCAACA,
z is 25;
(SEQ ID NO: 17)
GGACGATGATACTCAGCAAC,
z is 20;
(SEQ ID NO: 18)
GAGGACGATGATACTCAGCA,
z is 20;
(SEQ ID NO: 19)
TGGAGGACGATGATACTCAG,
z is 20;
(SEQ ID NO: 20)
CGACGTGGAGGACGATGATACTCAG,
z is 25;
(SEQ ID NO: 21)
CGTGGAGGACGATGATACTC,
z is 20;
(SEQ ID NO: 22)
GACGTGGAGGACGATGATAC,
z is 20;
(SEQ ID NO: 23)
CACCGCGACGTGGAGGACGATGATA,
z is 25;
(SEQ ID NO: 24)
GCGACGTGGAGGACGATGAT,
z is 20;
(SEQ ID NO: 25)
ACCAGCACCGCGACGTGGAGGACGA,
z is 25;
(SEQ ID NO: 26)
GCAGCACCAGCACCGCGACGTGGAG,
z is 25;
(SEQ ID NO: 27)
GAACAGCAGCACCAGCACCGCGACG,
z is 25;
(SEQ ID NO: 28)
GAGACGAACAGCAGCACCAGCACCG,
z is 25;
(SEQ ID NO: 29)
AGGCACTCACGCTGACGATCGTGGA,
z is 25;
(SEQ ID NO: 30)
CGATCCATTGCTAGAGAGAATCAGA,
z is 25;
(SEQ ID NO: 31)
CGTGTCCATTGCCCACGATCCATTG,
z is 25;
(SEQ ID NO: 32)
CCAGAGATCAGTTGCGTGTCCATTG,
z is 25;
(SEQ ID NO: 33)
ACAGTTCTGCCAGAGATCAGTTGCG,
z is 25;
(SEQ ID NO: 34)
GACATTTCCTGAGGAAGAGGTGCTA,
z is 25;
(SEQ ID NO: 35)
GATGAGAAACAGTGGTGGACATTTC,
z is 25;
(SEQ ID NO: 36)
TTTGGTGATGATGAGAAACAGTGGT,
z is 25;
(SEQ ID NO: 37)
AGCCTCACCGTTTGGTGATGATGAG,
z is 25;
(SEQ ID NO: 38)
CACCGTTTGGTGATGATGAGAAACA,
z is 25;
(SEQ ID NO: 39)
CAGACTGCAGCCATTCTGGGGGAAA,
z is 25;
(SEQ ID NO: 40)
GAATGCTGAAGATGATCGACAGGAT,
z is 25;
(SEQ ID NO: 41)
AGAGTTGGCAGAAGAACAGGAACAG,
z is 25;
(SEQ ID NO: 42)
TGTAAAACCTGCCCCCCTTGGTGAG,
z is 25;
(SEQ ID NO: 43)
ATTCCAGTGATGTAAAACCTGCCCC,
z is 25;
(SEQ ID NO: 44)
AATTTGGAAGATTCCAGTGATGTAA,
z is 25;
(SEQ ID NO: 45)
TACCAGCAAGAATTTGGAAGATTCC,
z is 25;
(SEQ ID NO: 46)
CACTCATCACGCACAGACCTGGGGAA,
z is 26;
(SEQ ID NO: 47)
GCCTCACCGTGTAGATGGCCGCAGC,
z is 25;
(SEQ ID NO: 48)
TTGAGATGCCACTCCGGGTGCCTCA,
z is 25;
(SEQ ID NO: 49)
CCGTAGGAGTAATCCGAGTTGAGAT,
z is 25;
(SEQ ID NO: 50)
CTCTGATGTTTATTTTAATGCATCT,
z is 25

30. The antisense oligomer of any one of claims 24-29, wherein the antisense oligomer is complementary to a portion of, or induces skipping of, exon 2.

31. The antisense oligomer of claim 30, wherein the target region is PMP22 H2A (−25−1), PMP22 H2A (+1+25), PMP22 H2A (+25+49), PMP22 H2A (+30+54), PMP22 H2A (+35+59), PMP22 H2A (+40+64), PMP22 H2A (+45+69), PMP22 H2A (+50+74), PMP22 H2A (+55+79), PMP22 H2A (+60+84), PMP22 H2A (+65+89), PMP22 H2A (+70+94), PMP22 H2A (+75+99), or PMP22 H2D (+15−10).

32. The antisense oligomer of claim 31, wherein the antisense oligomer comprises a targeting sequence selected from SEQ ID NOs: 6 to 29.

33. The antisense oligomer of any one of claims 24-29, wherein the antisense oligomer is complementary to a portion of, or induces skipping of, exon 3.

34. The antisense oligomer of claim 33, wherein the target region is PMP22 H3A (−15+10), PMP22 H3A (+1+25), PMP22 H3A (+15+39), PMP22 H3A (+24+48), PMP22 H3A (+48+72), PMP22 H3A (+65+89), PMP22 H3A (+74+98), PMP22 H3D (+17−8), or PMP22 H3D (+22−3).

35. The antisense oligomer of claim 34, wherein the antisense oligomer comprises a targeting sequence selected from SEQ ID NOs: 30 to 38.

36. The antisense oligomer of any one of claims 24-29, wherein the antisense oligomer is complementary to a portion of, or induces skipping of, exon 4.

37. The antisense oligomer of claim 36, wherein the target region is PMP22 H4A (−10+15), PMP22 H4A (+30+54), PMP22 H4A (+60+84), PMP22 H4A (+90+114), PMP22 H4A (+100+124), PMP22 H4A (+110+134), or PMP22 H4D (+22−3).

38. The antisense oligomer of claim 37, wherein the antisense oligomer comprises a targeting sequence selected from SEQ ID NOs: 39 to 45.

39. The antisense oligomer of any one of claims 24-29, wherein the antisense oligomer is complementary to a portion of, or induces skipping of, exon 5.

40. The antisense oligomer of claim 39, wherein the target region is PMP22 H5A (−8+17), PMP22 H5A (+18+42), PMP22 H5A (+37+61), PMP22 H5A (+55+79), or PMP22 H5A (+1271+1295).

41. The antisense oligomer of claim 40, wherein the antisense oligomer comprises a targeting sequence selected from SEQ ID NOs: 46 to 50.

42. The antisense oligomer of any one of claims 24-41, wherein the phosphorodiamidate morpholino oligomer is covalently linked to a cell-penetrating peptide, and wherein one of the following definitions occurs in the oligomer of Formula I:

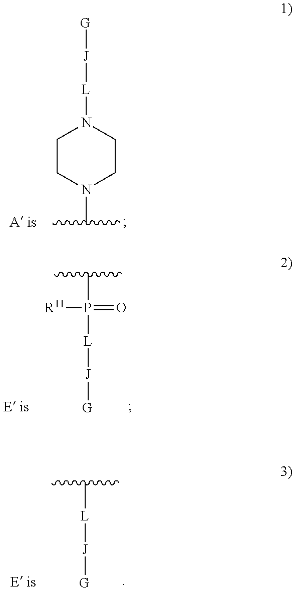

43. The antisense oligomer of any one of claims 24-41, wherein E′ is selected from H, —C₁₋₄-alkyl, —C(O)C₁₋₆-alkyl, benzoyl, stearoyl, trityl, monomethoxytrityl, dimethoxytrityl, trimethoxytrityl, and

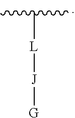

44. The antisense oligomer of any one of claims 24-41, wherein
A′ is selected from —N(C₁₋₆-alkyl)CH₂C(O)NH₂,

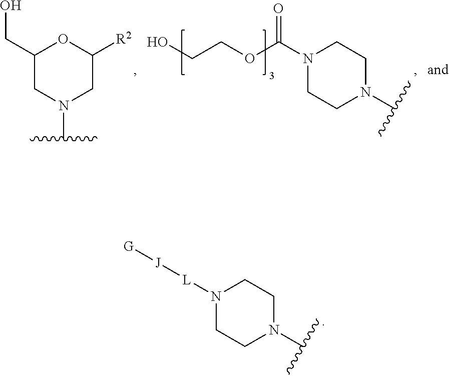

45. The antisense oligomer of any one of claims 24-41, wherein E′ is selected from H, —C(O)CH₃, benzoyl, stearoyl, trityl, 4-methoxytrityl, and

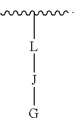

46. The antisense oligomer of any one of claims 24-41, wherein A′ is selected from —N(C₁₋₆-alkyl)CH₂C(O)NH₂,

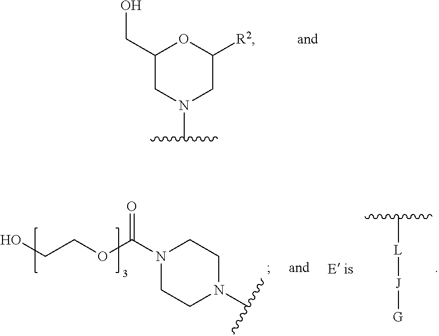

47. The antisense oligomer of any one of claims 24-41, wherein A′ is 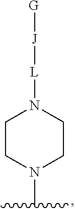
and
E′ is selected from H, —C(O)CH₃, trityl, 4-methoxytrityl, benzoyl, and stearoyl.

48. The antisense oligomer of any one of claims 24-41, wherein the peptide-oligonucleotide conjugate of Formula I is a peptide-oligonucleotide conjugate selected from: 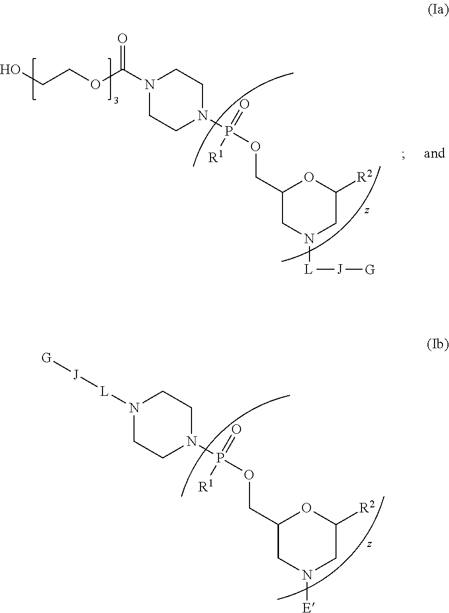
wherein E′ is selected from H, C₁₋₆-alkyl, —C(O)CH₃, benzoyl, and stearoyl.

49. The antisense oligomer of claim 48, wherein the peptide-oligonucleotide conjugate is of the formula (Ia).

50. The antisense oligomer of claim 48, wherein the peptide-oligonucleotide conjugate is of the formula (Ib).

51. The antisense oligomer of any one of claims 24-50, or a pharmaceutically acceptable salt thereof, wherein E′ is selected from —C(O)(alkyl)ᵥ(O-alkyl)ᵤ-NHC(O)—R⁹, —C(O)—R⁹, and —R⁹, wherein u is 0-12, v is 0-12, each alkyl group is, independently at each occurrence, C₂₋₆-alkyl.

52. The antisense oligomer of any one of claims 24-51, or a pharmaceutically acceptable salt thereof, wherein
A′ is 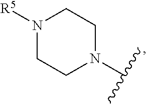
wherein R⁵ is selected from —C(O)(alkyl)_{w}(O-alkyl)_{y}-NHC(O)—R⁹, —C(O)—R⁹, and —R⁹, wherein y is 0-12, w is 0-12, each alkyl group is, independently at each occurrence, C₂₋₆-alkyl.

53. The antisense oligomer of any one of claims 24-52, or a pharmaceutically acceptable salt thereof, wherein E′ is —C(O)(alkyl)ᵥ(O-alkyl)ᵤ-NHC(O)—R⁹, wherein u is 0-12, v is 0-12, each alkyl group is, independently at each occurrence, C₂₋₆-alkyl.

54. The antisense oligomer of any one of claims 24-53, or a pharmaceutically acceptable salt thereof, wherein A′ is 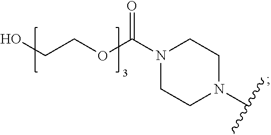
and
E′ is —C(O)(alkyl)ᵥ(O-alkyl)ᵤ-NHC(O)—R⁹, wherein u is 0-12, v is 0-12, each alkyl group is, independently at each occurrence, C₂₋₆-alkyl.

55. The antisense oligomer of any one of claims 24-54, or a pharmaceutically acceptable salt thereof, wherein A′ is —C(O)(alkyl)_{w}(O-alkyl)_{y}-NHC(O)—R⁹, wherein y is 0-12, w is 0-12, each alkyl group is, independently at each occurrence, C₂₋₆-alkyl; and E′ is selected from H, —C(O)CH₃, trityl, 4-methoxytrityl, benzoyl, and stearoyl.

56. The antisense oligomer of any one of claims 24-41, or a pharmaceutically acceptable salt thereof, wherein the conjugate of Formula I is a conjugate selected from: 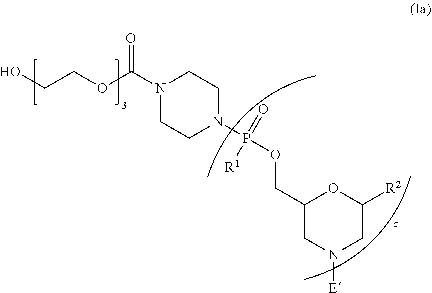
wherein E′ is —C(O)(alkyl)ᵥ(O-alkyl)ᵤ-NHC(O)—R⁹, wherein u is 0-12, v is 0-12, each alkyl group is, independently at each occurrence, C₂₋₆-alkyl; 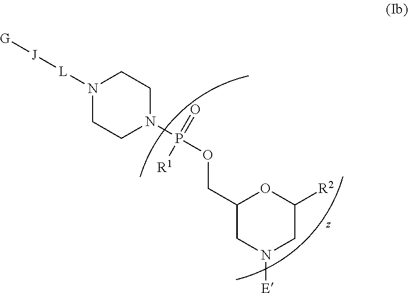
wherein E′ is —C(O)(alkyl)ᵥ(O-alkyl)ᵤ-NHC(O)—R⁹, wherein u is 0-12, v is 0-12, each alkyl group is, independently at each occurrence, C₂₋₆-alkyl; 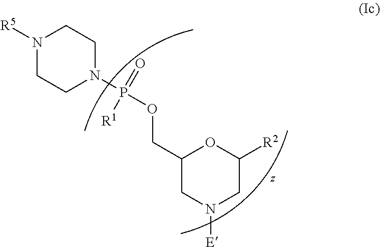
wherein R⁵ is selected from —C(O)(alkyl)_{w}(O-alkyl)_{y}-NHC(O)—R⁹, —C(O)—R⁹, and —R⁹, wherein y is 0-12, w is 0-12, each alkyl group is, independently at each occurrence, C₂₋₆-alkyl, and wherein E′ is selected from H, C₁₋₈-alkyl, —C(O)CH₃, benzoyl, and stearoyl; and 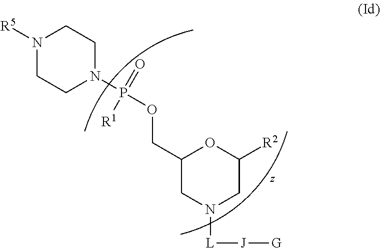
wherein R⁵ is selected from —C(O)(alkyl)_{w}(O-alkyl)_{y}-NHC(O)—R⁹, —C(O)—R⁹, and —R⁹, wherein y is 0-12, w is 0-12, each alkyl group is, independently at each occurrence, C₂₋₆-alkyl.

57. The antisense oligomer of claim 56, or a pharmaceutically acceptable salt thereof, wherein the conjugate is of the formula (Ic):

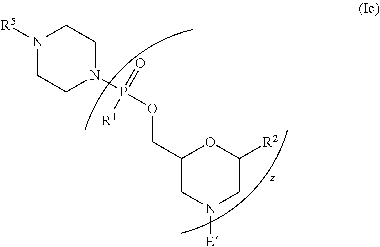

58. The antisense oligomer of claim 56, or a pharmaceutically acceptable salt thereof, wherein the conjugate is of the formula (Id):

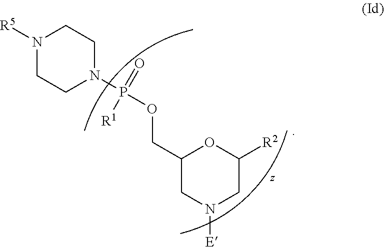

59. The antisense oligomer of any one of claims 24-58, or a pharmaceutically acceptable salt thereof, wherein the cell-penetrating peptide is selected from rTAT, Tat, R₉F₂, R₅F₂R₄, R₄, R₅, R₆, R₇, R₈, R₉, (RAhxR)₄, (RAhxR)₅, (RAhxRRBR)₂, (RAR)₄F₂, and (RGR)₄F₂.

60. The antisense oligomer of any one of claims 24-59, or a pharmaceutically acceptable salt thereof, wherein each R¹ is N(CH₃)₂.

61. The antisense oligomer of any one of claims 24-60, or a pharmaceutically acceptable salt thereof, wherein each R² is a nucleobase, independently at each occurrence, selected from adenine, guanine, cytosine, 5-methyl-cytosine, thymine, uracil, and hypoxanthine.

62. The antisense oligomer of any one of claims 24-61, or a pharmaceutically acceptable salt thereof, wherein L is glycine.

63. The antisense oligomer of any one of claims 24-62, or a pharmaceutically acceptable salt thereof, wherein G is selected from H, C(O)CH₃, benzoyl, and stearoyl.

64. The antisense oligomer of any one of claims 24-63, or a pharmaceutically acceptable salt thereof, wherein G is H or —C(O)CH₃.

65. The antisense oligomer of any one of claims 24-64, or a pharmaceutically acceptable salt thereof, wherein G is H.

66. The antisense oligomer of any one of claims 24-65, or a pharmaceutically acceptable salt thereof, wherein G is —C(O)CH₃.

67. A pharmaceutical composition comprising the oligomer of any one of claims 1-66 and a pharmaceutically acceptable carrier.

68. A compound of any one of claims 1-66 or a composition of claim 67 for use in treating a disease associated with dysregulation of peripheral myelin protein 22 in a subject in need thereof.

69. The compound or composition for use according to claim 68, wherein the disease associated with dysregulation of peripheral myelin protein 22 is Charcot-Marie-Tooth type 1A (CMT1A).

70. A method of treating a disease associated with dysregulation of peripheral myelin protein 22, comprising administering to a patient in need thereof a therapeutically effective amount of the antisense oligomer of any one of claims 1-66 or the pharmaceutical composition of claim 67.

71. The method of claim 70, wherein the disease associated with dysregulation of peripheral myelin protein 22 is Charcot-Marie-Tooth type 1A (CMT1A).

72. A compound of any one of claims 1-66 or a composition of claim 67 for use as a medicament.

73. A compound of any one of claims 1-66 or a composition of claim 67 for use in the manufacture of a medicament for treatment of a disease associated with dysregulation of peripheral myelin protein 22 in a subject in need thereof.

74. The compound or composition for use according to claim 73, wherein the disease associated with dysregulation of peripheral myelin protein 22 is Charcot-Marie-Tooth type 1A (CMT1A).

75. The compound or composition for use according to claim 74, wherein the disease is Charcot-Marie-Tooth type 1 A neuropathy.

76. A method of reducing peripheral myelin protein 22 expression in a patient in need thereof, comprising administering to the patient a therapeutically effective amount of the antisense oligomer of any one of claims 1-66.

77. The method of claim 76, wherein the patient has a disease associated with dysregulation of peripheral myelin protein 22.

78. The method of claim 76-77, wherein the patient has Charcot-Marie-Tooth disease type 1A.
